SEQUENCE LISTING (10 sequences)

SEQ ID NO: 1 - DNA, 54 bp, Artificial Sequence
  source: /note=“Description of Artificial Sequence Synthetic primer”
  modified_base (7)..(24): a, c, t, g, unknown or other
cagagcnnnnnnnnnnnnnnnnnntttttttttttttttttttttttttttttt

SEQ ID NO: 2 - DNA, 45 bp, Artificial Sequence
  source: /note=“Description of Artificial Sequence Synthetic oligonucleotide”
  source: /note=“Description of Combined DNA/RNA Molecule Synthetic oligonucleotide”
  modified_base (7)..(16): a, c, t, g, unknown or other
  misc_feature (7)..(16): /note=“This region may encompass 9-10 nucleotides”
  modified_base (36)..(45): a, c, t, g, unknown or other
  misc_feature (36)..(45): /note=“This region may encompass 9-10 nucleotides”
  source: /note=“See specification as filed for detailed description of substitutions and preferred embodiments”
gctctgnnnnnnnnnnuacgacgctcttccgatctnnnnnnnnnn

SEQ ID NO: 3 - DNA, 29 bp, Artificial Sequence
  source: /note=“Description of Artificial Sequence Synthetic primer”
aatgatacggcgaccaccgagatctacac

SEQ ID NO: 4 - DNA, 24 bp, Artificial Sequence
  source: /note=“Description of Artificial Sequence Synthetic primer”
caagcagaagacggcatacgagat

SEQ ID NO: 5 - DNA, 33 bp, Artificial Sequence
  source: /note=“Description of Artificial Sequence Synthetic primer”
tcgtcggcagcgtcagatgtgtataagagacag

SEQ ID NO: 6 - DNA, 34 bp, Artificial Sequence
  source: /note=“Description of Artificial Sequence Synthetic primer”
gtctcgtgggctcggagatgtgtataagagacag

SEQ ID NO: 7 - DNA, 25 bp, Artificial Sequence
  source: /note=“Description of Artificial Sequence Synthetic primer”
ctccgagcccacgagacgacaagtc

SEQ ID NO: 8 - DNA, 30 bp, Artificial Sequence
  source: /note=“Description of Artificial Sequence Synthetic primer”
acacatctgacgctgccgacgactgattac

SEQ ID NO: 9 - DNA, 33 bp, Artificial Sequence
  source: /note=“Description of Artificial Sequence Synthetic primer”
acactctttccctacacgacgctcttccgatct

SEQ ID NO: 10 - DNA, 15 bp, Artificial Sequence
  source: /note=“Description of Artificial Sequence Synthetic primer”
gtctcgtgggctcgg

(19) United States
(12) Patent Application Publication
(10) Pub. No.: US 2022/0356461 A1
(43) Pub. Date: Nov. 10, 2022

(54) HIGH-THROUGHPUT SINGLE-CELL LIBRARIES AND METHODS OF MAKING AND OF USING

(71) Applicants: Illumina, Inc., San Diego, CA (US); University of Washington, Seattle, WA (US)

(72) Inventors: Jay Shendure, Seattle, WA (US); Darren Cusanovich, Seattle, WA (US); Riza Daza, Seattle, WA (US); Frank Steemers, San Diego, CA (US); Andrew Kennedy, San Diego, CA (US)

(73) Assignees: Illumina, Inc., San Diego, CA (US); University of Washington, Seattle, WA (US)

(21) Appl. No.: 17/441,741

(22) PCT Filed: Dec. 18, 2020
(86) PCT No.: PCT/US2020/066013

§ 371 Date: Sep. 22, 2021

Related U.S. Application Data
(60) Provisional application No. 62/950,670, filed on Dec. 19, 2019.

(51) Int. Cl. C12N 15/10; C12Q 1/6874; C12Q 1/6806

(52) U.S. Cl. CPC C12N 15/1065; C12N 15/1082; C12Q 1/6874; C12Q 1/6806

(57) ABSTRACT

Provided herein are methods for preparing a sequencing library that includes nucleic acids from a plurality of single cells. In one embodiment, the sequencing library includes nucleic acids that represent the chromatin accessibility from the plurality of single cells. In one embodiment, the nucleic acids include three index sequences. In another embodiment, the present disclosure provides methods for characterizing rare events in isolated cells and nuclei.

### CROSS-REFERENCE TO RELATED APPLICATIONS

This application claims the benefit of U.S. Provisional Application Ser. No. 62/950,670, filed Dec. 19, 2019, which is incorporated by reference herein in its entirety

### GOVERNMENT FUNDING

This invention was made with government support under Grant No. T32 HL007828, awarded by the National Institutes of Health. The government has certain rights in the invention.

### FIELD

Embodiments of the present disclosure relate to sequencing nucleic acids. In particular, embodiments of the methods and compositions provided herein relate to producing single-cell combinatorial indexed sequencing libraries and obtaining sequence data therefrom. In some embodiments, the sequence data obtained from the libraries is comprehensive, and in other embodiments the sequence data obtained from the libraries permits characterization of rare events.

### BACKGROUND

Single cell combinatorial indexing (‘sci-’) is a methodological framework that employs split-pool barcoding to uniquely label the nucleic acid contents of large numbers of single cells or nuclei to produce single-cell combinatorial sequencing libraries. Current single cell genomic techniques often include the use of a transposome complex to add the unique label at one step; however, this requires a large quantity of custom modified transposons.

Single cell genomic techniques resolve cellular differences that are difficult to determine when studying bulk population of cells. In many important applications such as oncology, immunology, and metagenomics there is great interest and challenge in characterizing rare cells. Current methods in single-cell sequencing enable the characterization of millions of single-cells in parallel; however, comprehensive sequencing-based characterization of rare cells in a population without enrichment is costly and challenging.

### SUMMARY

Provided herein are methods to use a transposome complex during single-cell combinatorial indexing without the need to produce the custom modified transposons.

In one embodiment, the present disclosure provides a method for preparing a sequencing library that includes nucleic acids from a plurality of single nuclei or cells. The method includes providing a plurality of nuclei or cells, where the nuclei or cells include nucleosomes and contacting the plurality of nuclei or cells with a transposome complex that includes a transposase and a universal sequence. In one embodiment, the plurality of nuclei or cells are in bulk when contacted with the transposome complex, and in another embodiment when contacted with the transposome complex the plurality of nuclei or cells are distributed in a first plurality of compartments, where each compartment includes a subset of nuclei or cells or represents a sample. The contacting further includes conditions suitable for incorporation of the universal sequence into DNA nucleic acids resulting in double stranded DNA nucleic acids that include the universal sequence. In those embodiments where the contacting occurs with the plurality of nuclei or cells are in bulk, the method also includes distributing the plurality of nuclei or cells into a first plurality of compartments, where each compartment includes a subset of nuclei or cells. The DNA molecules in each subset of nuclei or cells are processed to generate indexed nuclei or cells. The processing includes adding to DNA nucleic acids present in each subset of nuclei or cells a first compartment specific index sequence to result in indexed nucleic acids present in indexed nuclei or cells. The processing can include ligation, primer extension, hybridization, amplification, or a combination thereof. The indexed nuclei or cells can be combined to generate pooled indexed nuclei or cells.

In one embodiment, the providing can include providing the plurality of nuclei or cells in a plurality of compartments, where each compartment includes a subset of nuclei or cells or represents a sample. The contacting can include contacting each compartment with the transposome complex, and the method can further include combining the nuclei or cells after the contacting to generate pooled nuclei or cells.

In one embodiment, the contacting includes contacting each subset with two transposome complexes, where one transposome complex includes a first transposase including a first universal sequence and a second transposome complex includes a second transposase including a second universal sequence, wherein the contacting further includes conditions suitable for incorporation of the first universal sequence and the second universal sequence into DNA nucleic acids resulting in double stranded DNA nucleic acids including the first and second universal sequences.

In one embodiment, the method can further include distributing the pooled indexed nuclei or cells that include the indexed nuclei or cells into a second plurality of compartments where each compartment includes a subset of nuclei or cells, and processing DNA molecules in each subset of nuclei or cells to generate dual-indexed nuclei or cells. The processing can include adding to DNA nucleic acids present in each subset of nuclei or cells a second compartment specific index sequence to result in dual-indexed nucleic acids present in indexed nuclei or cells. The method can include combining the dual-indexed nuclei or cells to generate pooled dual-indexed nuclei or cells.

In one embodiment, the method can further include distributing the pooled indexed nuclei or cells that include the dual-indexed nuclei or cells into a third plurality of compartments where each compartment includes a subset of nuclei or cells, and processing DNA molecules in each subset of nuclei or cells to generate triple-indexed nuclei or cells. The processing can include adding to DNA nucleic acids present in each subset of nuclei or cells a third compartment specific index sequence to result in triple-indexed nucleic acids present in indexed nuclei or cells. The method can include combining the triple-indexed nuclei or cells to generate pooled triple-indexed nuclei or cells.

In one embodiment, the method can further include obtaining the indexed nucleic acids (e.g., dual-indexed, triple-indexed, etc.) from the pooled indexed nuclei or cells, thereby producing a sequencing library from the plurality of nuclei or cells.

Also provided herein are methods to identify and/or characterize a subpopulation of cells. In one embodiment, the method includes providing a sequencing library, such as a single-cell combinatorial sequencing library. Optionally, the sequencing library is produced from a population of cells or nuclei that are enriched for a characteristic. The method can include interrogating the sequencing library by targeted sequencing. The targeted sequencing can be based on a biological feature that is typically present in a small percentage of the cells used to make the library. Examples of a biological feature include, but are not limited to, a nucleotide sequence indicative of cell class, species type, or disease state. In addition to the targeted sequencing of the biological feature, the sequencing also includes determining the sequence of the index sequences that are present on the same modified target nucleic acid as the biological feature. The result is the identification of the members of the sequencing library that originate from the same cells or nuclei as the members of the library that include the biological feature. The method further includes altering the sequencing library to increase the representation of those members that originate from the same cells or nuclei as the members of the library that include the biological feature. The alteration can include enrichment of the desired members of the sequencing library, or depletion of the undesirable members of the sequencing library, to result in a sub-library.

Definitions

Terms used herein will be understood to take on their ordinary meaning in the relevant art unless specified otherwise. Several terms used herein and their meanings are set forth below.

As used herein, the terms “organism,” “subject,” are used interchangeably and refer to microbes (e.g., prokaryotic or eukaryotic), animals, and plants. An example of an animal is a mammal, such as a human.

As used herein, the term “cell type” is intended to identify cells based on morphology, phenotype, developmental origin or other known or recognizable distinguishing cellular characteristic. A variety of different cell types can be obtained from a single organism (or from the same species of organism). Exemplary cell types include, but are not limited to, gametes (including female gametes, e.g., ova or egg cells, and male gametes, e.g., sperm), ovary epithelial, ovary fibroblast, testicular, urinary bladder, immune cells, B cells, T cells, natural killer cells, dendritic cells, cancer cells, eukaryotic cells, stem cells, blood cells, muscle cells, fat cells, skin cells, nerve cells, bone cells, pancreatic cells, endothelial cells, pancreatic epithelial, pancreatic alpha, pancreatic beta, pancreatic endothelial, bone marrow lymphoblast, bone marrow B lymphoblast, bone marrow macrophage, bone marrow erythroblast, bone marrow dendritic, bone marrow adipocyte, bone marrow osteocyte, bone marrow chondrocyte, promyeloblast, bone marrow megakaryoblast, bladder, brain B lymphocyte, brain glial, neuron, brain astrocyte, neuroectoderm, brain macrophage, brain microglia, brain epithelial, cortical neuron, brain fibroblast, breast epithelial, colon epithelial, colon B lymphocyte, mammary epithelial, mammary myoepithelial, mammary fibroblast, colon enterocyte, cervix epithelial, breast duct epithelial, tongue epithelial, tonsil dendritic, tonsil B lymphocyte, peripheral blood lymphoblast, peripheral blood T lymphoblast, peripheral blood cutaneous T lymphocyte, peripheral blood natural killer, peripheral blood B lymphoblast, peripheral blood monocyte, peripheral blood myeloblast, peripheral blood monoblast, peripheral blood promyeloblast, peripheral blood macrophage, peripheral blood basophil, liver endothelial, liver mast, liver epithelial, liver B lymphocyte, spleen endothelial, spleen epithelial, spleen B lymphocyte, liver hepatocyte, liver, fibroblast, lung epithelial, bronchus epithelial, lung fibroblast, lung B lymphocyte, lung Schwann, lung squamous, lung macrophage, lung osteoblast, neuroendocrine, lung alveolar, stomach epithelial, and stomach fibroblast. In one embodiment, a variety of different cell types obtained from a single organism can include the organism's cells and other cells such as cells of commensal or pathogenic microbes associated with the organism. Examples of commensal or pathogenic microbes associated with the organism include, but are not limited to, prokaryotic and eukaryotic microbes present in a microbiome sample from the organism or present in a tissue and optionally causing disease.

As used herein, the term “tissue” is intended to mean a collection or aggregation of cells that act together to perform one or more specific functions in an organism. The cells can optionally be morphologically similar. Exemplary tissues include, but are not limited to, embryonic, epididymitis, eye, muscle, skin, tendon, vein, artery, blood, heart, spleen, lymph node, bone, bone marrow, lung, bronchi, trachea, gut, small intestine, large intestine, colon, rectum, salivary gland, tongue, gall bladder, appendix, liver, pancreas, brain, stomach, skin, kidney, ureter, bladder, urethra, gonad, testicle, ovary, uterus, fallopian tube, thymus, pituitary, thyroid, adrenal, or parathyroid. Tissue can be derived from any of a variety of organs of a human or other organism. A tissue can be a healthy tissue or an unhealthy tissue. Examples of unhealthy tissues include, but are not limited to, malignancies in reproductive tissue, lung, breast, colorectum, prostate, nasopharynx, stomach, testes, skin, nervous system, bone, ovary, liver, hematologic tissues, pancreas, uterus, kidney, lymphoid tissues, etc. The malignancies may be of a variety of histological subtypes, for example, carcinoma, adenocarcinoma, sarcoma, fibroadenocarcinoma, neuroendocrine, or undifferentiated.

As defined herein, “sample” and its derivatives, is used in its broadest sense and includes any specimen, culture and the like that is suspected of including a target nucleic acid and/or a target protein. In some embodiments, the sample comprises DNA, RNA, protein, or a combination thereof. The sample can include any biological, clinical, surgical, agricultural, atmospheric or aquatic-based specimen containing one or more nucleic acids and/or one or more proteins. The term also includes any isolated nucleic acid from sample such a genomic DNA or a transcriptome, and any isolated protein from a sample. In some embodiments, the sample includes a collection of cells or nuclei.

As used herein, the term “compartment” is intended to mean an area or volume that separates or isolates something from other things. Exemplary compartments include, but are not limited to, vials, tubes, wells, droplets, boluses, beads, vessels, surface features, or areas or volumes separated by physical forces such as fluid flow, magnetism, electrical current or the like. In one embodiment, a compartment is a well of a multi-well plate, such as a 96- or 384-well plate. In one embodiment, a compartment is a well (e.g., a microwell or a nanowell) of a patterned surface. As used herein, a droplet may include a hydrogel bead, which is a bead for encapsulating one or more nuclei or cells and includes a hydrogel composition. In some embodiments, the droplet is a homogeneous droplet of hydrogel material or is a hollow droplet having a polymer hydrogel shell. Whether homogenous or hollow, a droplet may be capable of encapsulating one or more nuclei or cells. In some embodiments, the droplet is a surfactant stabilized droplet.

As used herein, a “transposome complex” refers to an integration enzyme and a nucleic acid including an integration recognition site. A “transposome complex” is a functional complex formed by a transposase and a transposase recognition site that is capable of catalyzing a transposition reaction (see, for instance, Gunderson et al., WO 2016/130704). Examples of integration enzymes include, but are not limited to, an integrase or a transposase. Examples of integration recognition sites include, but are not limited to, a transposase recognition site.

As used herein, the term “nucleic acid” is used interchangeably with polynucleotide and oligonucleotide. Nucleic acid is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of adenine, uracil, cytosine or guanine. Useful non-native bases that can be included in a nucleic acid are known in the art. Examples of non-native bases include a locked nucleic acid (LNA), a bridged nucleic acid (BNA), and pseudo-complementary bases (Trilink Biotechnologies, San Diego, Calif.). LNA and BNA bases can be incorporated into a DNA oligonucleotide and increase oligonucleotide hybridization strength and specificity. LNA and BNA bases and the uses of such bases are known to the person skilled in the art and are routine. Unless indicated otherwise, the term “nucleic acid” includes natural and non-natural DNA, mRNA, and non-coding RNA, e.g., RNA without poly-A at 3′ end, and nucleic acids derived from a RNA, e.g., cDNA. The term “nucleic acid” refers only to the primary structure of the molecule. Thus, the term includes triple-, double- and single-stranded deoxyribonucleic acid (“DNA”), as well as triple-, double- and single-stranded ribonucleic acid (“RNA”).

As used herein, the term “target,” is intended as a semantic identifier for a molecule whose source, function, identity, and/or composition is being investigated. Examples of targets include, but are not limited to, nucleic acid and protein. As used herein, the term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated. A target nucleic acid may be essentially any nucleic acid of known or unknown sequence. It may be, for example, a fragment of genomic DNA (e.g., chromosomal DNA), extra-chromosomal DNA such as a plasmid, cell-free DNA, RNA (e.g., RNA or non-coding RNA), proteins (e.g., cellular or cell surface proteins), or cDNA. A target nucleic acid may be a nucleic acid that is attached to a compound, such as an antibody, that specifically binds a biomolecule, such as a protein, glycan, proteoglycan, or lipid (U.S. Application Pub2018/0273933). Sequencing may result in determination of the sequence of the whole, or a part of the target molecule. The targets can be derived from a primary nucleic acid sample, such as a nucleus. In one embodiment, the targets can be processed into templates suitable for amplification by the placement of universal sequences at one or both ends of each target fragment. The targets can also be obtained from a primary RNA sample by reverse transcription into cDNA. In one embodiment, target is used in reference to a subset of DNA, RNA, or proteins present in the cell. Targeted sequencing uses selection and isolation of genes or regions or proteins of interest, typically by either PCR amplification (e.g., region-specific primers) or hybridization-based capture method or antibodies. Targeted enrichment can occur at various stages of the method. For instance, a targeted RNA representation can be obtained using target specific primers in a reverse transcription step or hybridization-based enrichment of a subset out of a more complex library. An example is exome sequencing or the L1000 assay (Subramanian et al., 2017, Cell, 171; 1437-1452). Targeted sequencing can include any of the enrichment processes known to one of ordinary skill in the art. A target nucleic acid having a universal sequence one or both ends can be referred to as a modified target nucleic acid. Reference to a nucleic acid such as a target nucleic acid includes both single stranded and double stranded nucleic acids unless indicated otherwise. In one embodiment, libraries are enriched using the index sequence or index sequences. In some embodiments, the enrichment involves one or more index sequences attached to the same library molecule, e.g., introduced through combinatorial indexing.

As used herein, the term “universal,” when used to describe a nucleotide sequence, refers to a region of sequence that is common to two or more nucleic acid molecules where the molecules also have regions of sequence that differ from each other. A universal sequence that is present in different members of a collection of molecules, e.g., members of a sequencing library, can allow capture of multiple different nucleic acids using a population of universal capture sequences. Non-limiting examples of universal capture sequences include sequences that are identical to or complementary to P5 and P7 primers. Similarly, a universal sequence present in different members of a collection of molecules can allow the replication (e.g., sequencing) or amplification of multiple different nucleic acids using a population of universal primers that are complementary to a portion of the universal sequence, e.g., a universal primer binding site. The terms “A14” and “B15” may be used when referring to a universal primer binding site. The terms “A14′” (A14 prime) and “B15′” (B15 prime) refer to the complement of A14 and B15, respectively. It will be understood that any suitable universal primer binding site can be used in the methods presented herein, and that the use of A14 and B15 are exemplary embodiments only. In one embodiment, a universal primer binding site is used as a site to which a universal primer (e.g., a sequencing primer for read 1 or read 2) anneals for sequencing.

The terms “P5” and “P7” may be used when referring to a universal capture sequence or a capture oligonucleotide. The terms “P5′” (P5 prime) and “P7′” (P7 prime) refer to the complement of P5 and P7, respectively. It will be understood that any suitable universal capture sequence or a capture oligonucleotide can be used in the methods presented herein, and that the use of P5 and P7 are exemplary embodiments only. Uses of capture oligonucleotides such as P5 and P7 or their complements on flowcells are known in the art, as exemplified by the disclosures of WO 2007/010251, WO 2006/064199, WO 2005/065814, WO 2015/106941, WO 1998/044151, and WO 2000/018957. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein.

As used herein, the term “primer” and its derivatives refer generally to any nucleic acid that can hybridize to a sequence of interest. Typically, the primer functions as a substrate onto which nucleotides can be polymerized by a polymerase or to which a nucleotide sequence such as an index can be ligated; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. A primer can be a nucleic acid that is single-stranded, double-stranded, or include a single-stranded region(s) and a double-stranded region(s), and may include ribonucleotides, deoxyribonucleotides, analogs thereof, or mixtures thereof. The terms “polynucleotide” and “oligonucleotide” are used interchangeably herein. The terms should be understood to include, as equivalents, analogs of either DNA, RNA, cDNA or antibody-oligo conjugates made from nucleotide analogs and to be applicable to single stranded (such as sense or antisense) and double stranded polynucleotides. The term as used herein also encompasses cDNA, that is complementary or copy DNA produced from a RNA template, for example by the action of reverse transcriptase. This term refers only to the primary structure of the molecule. Thus, the term includes triple-, double- and single-stranded deoxyribonucleic acid (“DNA”), as well as triple-, double- and single-stranded ribonucleic acid (“RNA”).

As used herein, the term “adapter” and its derivatives, e.g., universal adapter, refers generally to any linear oligonucleotide which can be attached to a nucleic acid molecule of the disclosure. In some embodiments, the adapter is substantially non-complementary to the 3′ end or the 5′ end of any target sequence present in the sample. In some embodiments, suitable adapter lengths are in the range of about 10-100 nucleotides, about 12-60 nucleotides, or about 15-50 nucleotides in length. Generally, the adapter can include any combination of nucleotides and/or nucleic acids. In some aspects, the adapter can include one or more cleavable groups at one or more locations. In another aspect, the adapter can include a sequence that is substantially identical, or substantially complementary, to at least a portion of a primer, for example a universal primer. In some embodiments, the adapter can include a barcode (also referred to herein as a tag or index) to assist with downstream error correction, identification, or sequencing. The terms “adaptor” and “adapter” are used interchangeably.

As used herein, the term “each,” when used in reference to a collection of items, is intended to identify an individual item in the collection but does not necessarily refer to every item in the collection unless the context clearly dictates otherwise.

As used herein, the term “transport” refers to movement of a molecule through a fluid. The term can include passive transport such as movement of molecules along their concentration gradient (e.g. passive diffusion). The term can also include active transport whereby molecules can move along their concentration gradient or against their concentration gradient. Thus, transport can include applying energy to move one or more molecule in a desired direction or to a desired location such as an amplification site.

As used herein, “amplify,” “amplifying,” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

As used herein, “amplification conditions” and its derivatives, generally refers to conditions suitable for amplifying one or more nucleic acid sequences. Such amplification can be linear or exponential. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences flanked by a universal sequence, or to amplify an amplified target sequence ligated to one or more adapters. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending and separating are repeated. Typically, the amplification conditions include cations such as Mg²⁺ or Mn²⁺ and can also include various modifiers of ionic strength.

As used herein, “re-amplification” and their derivatives refer generally to any process whereby at least a portion of an amplified nucleic acid molecule is further amplified via any suitable amplification process (referred to in some embodiments as a “secondary” amplification), thereby producing a reamplified nucleic acid molecule. The secondary amplification need not be identical to the original amplification process whereby the amplified nucleic acid molecule was produced; nor need the reamplified nucleic acid molecule be completely identical or completely complementary to the amplified nucleic acid molecule; all that is required is that the reamplified nucleic acid molecule include at least a portion of the amplified nucleic acid molecule or its complement. For example, the re-amplification can involve the use of different amplification conditions and/or different primers, including different target-specific primers than the primary amplification.

As used herein, the term “polymerase chain reaction” (“PCR”) refers to the method of Mullis (U.S. Pat. Nos. 4,683,195 and 4,683,202), which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as PCR. Because the desired amplified segments of the polynucleotide of interest become the predominant nucleic acid sequences (in terms of concentration) in the mixture, they are said to be “PCR amplified”. In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

As defined herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexy” or “plex” of a given multiplex amplification refers generally to the number of different target-specific sequences that are amplified during that single multiplex amplification. In some embodiments, the plexy can be about 12-plex, 24-plex, 48-plex, 96-plex, 192-plex, 384-plex, 768-plex, 1536-plex, 3072-plex, 6144-plex or higher. It is also possible to detect the amplified target sequences by several different methodologies (e.g., gel electrophoresis followed by densitometry, quantitation with a bioanalyzer or quantitative PCR, hybridization with a labeled probe; incorporation of biotinylated primers followed by avidin-enzyme conjugate detection; incorporation of ³²P-labeled deoxynucleotide triphosphates into the amplified target sequence).

As used herein, “amplified target sequences” and its derivatives, refers generally to a polynucleotide sequence produced by the amplifying the target sequences using target-specific primers and the methods provided herein. The amplified target sequences may be either of the same sense (i.e., the positive strand) or antisense (i.e., the negative strand) with respect to the target sequences.

As used herein, the terms “ligating,” “ligation,” and their derivatives refer generally to the process for covalently linking two or more molecules together, for example covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, the ligation can include forming a covalent bond between a 5′ phosphate group of one nucleic acid and a 3′ hydroxyl group of a second nucleic acid thereby forming a ligated nucleic acid molecule. Generally, for the purposes of this disclosure, an amplified target sequence can be ligated to an adapter to generate an adapter-ligated amplified target sequence.

As used herein, “ligase” and its derivatives, refers generally to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases may include, but are not limited to, T4 DNA ligase, T4 RNA ligase, and E. coli DNA ligase.

As used herein, “ligation conditions” and its derivatives, generally refers to conditions suitable for ligating two molecules to each other. In some embodiments, the ligation conditions are suitable for sealing nicks or gaps between nucleic acids. As used herein, the term nick or gap is consistent with the use of the term in the art. Typically, a nick or gap can be ligated in the presence of an enzyme, such as ligase at an appropriate temperature and pH. In some embodiments, T4 DNA ligase can join a nick between nucleic acids at a temperature of about 70-72° C.

The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. An amplicon can be a nucleic acid molecule having a single copy of a particular nucleotide sequence (e.g. a PCR product) or multiple copies of the nucleotide sequence (e.g. a concatameric product of RCA). A first amplicon of a target nucleic acid is typically a complementary copy. Subsequent amplicons are copies that are created, after generation of the first amplicon, from the target nucleic acid or from the first amplicon.

As used herein, the term “amplification site” refers to a site in or on an array where one or more amplicons can be generated. An amplification site can be further configured to contain, hold or attach at least one amplicon that is generated at the site.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include, without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “capacity,” when used in reference to a site and nucleic acid material, means the maximum amount of nucleic acid material that can occupy the site. For example, the term can refer to the total number of nucleic acid molecules that can occupy the site in a particular condition. Other measures can be used as well including, for example, the total mass of nucleic acid material or the total number of copies of a particular nucleotide sequence that can occupy the site in a particular condition. Typically, the capacity of a site for a target nucleic acid will be substantially equivalent to the capacity of the site for amplicons of the target nucleic acid.

As used herein, the term “capture agent” refers to a material, chemical, molecule or moiety thereof that is capable of attaching, retaining or binding to a target molecule (e.g. a target nucleic acid). Exemplary capture agents include, without limitation, a capture sequence (also referred to herein as a capture oligonucleotide) that is complementary to at least a portion of a target nucleic acid, a member of a receptor-ligand binding pair (e.g. avidin, streptavidin, biotin, lectin, carbohydrate, nucleic acid binding protein, epitope, antibody, etc.) capable of binding to a target nucleic acid (or linking moiety attached thereto), or a chemical reagent capable of forming a covalent bond with a target nucleic acid (or linking moiety attached thereto).

As used herein, the term “reporter moiety” can refer to any identifiable tag, label, index, barcode, or group that enables to determine the composition, identity, and/or the source of a target that is investigated. In some embodiments, a reporter moiety may include an antibody that specifically binds to a protein. In some embodiments, the antibody may include a detectable label. In some embodiments, the reporter can include an antibody or affinity reagent labeled with a nucleic acid tag. In one embodiment, the nucleic acid is of sufficient length to serve as a substrate of a transposome complex. In one embodiment, the nucleic acid tag can be detectable, for example, via a proximity ligation assay (PLA) or proximity extension assay (PEA), sequencing-based readout (Shahi et al. Scientific Reports volume 7, Article number: 44447, 2017), or an epitope-based readout such as CITE-seq (Stoeckius et al. Nature Methods 14:865-868, 2017).

As used herein, the term “clonal population” refers to a population of nucleic acids that is homogeneous with respect to a particular nucleotide sequence. The homogenous sequence is typically at least 10 nucleotides long, but can be even longer including for example, at least 50, 100, 250, 500 or 1000 nucleotides long. A clonal population can be derived from a single target nucleic acid or template nucleic acid. Typically, all of the nucleic acids in a clonal population will have the same nucleotide sequence. It will be understood that a small number of mutations (e.g. due to amplification artifacts) can occur in a clonal population without departing from clonality.

As used herein, the term “unique molecular identifier” or “UMI” refers to a molecular tag, either random, non-random, or semi-random, that may be attached to a nucleic acid. When incorporated into a nucleic acid, a UMI can be used to correct for subsequent amplification bias by directly counting unique molecular identifiers (UMIs) that are sequenced after amplification.

As used herein, an “exogenous” compound, e.g., an exogenous enzyme, refers to a compound that is not normally or naturally found in particular composition. For instance, when the particular composition includes a cell lysate, an exogenous enzyme is an enzyme that is not normally or naturally found in the cell lysate.

As used herein, “providing” in the context of, for instance, a composition, an article, a nucleic acid, or a nucleus means making the composition, article, nucleic acid, or nucleus, purchasing the composition, article, nucleic acid, or nucleus, or otherwise obtaining the compound, composition, article, or nucleus.

The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

### BRIEF DESCRIPTION OF THE FIGURES

The following detailed description of illustrative embodiments of the present disclosure may be best understood when read in conjunction with the following drawings.

FIGS. 1A and 1B show general block diagrams of different embodiments of a general illustrative method for single-cell combinatorial indexing according to the present disclosure.

FIG. 2 shows a schematic drawing of a method for single-cell combinatorial indexing as generally illustrated in the method of FIG. 1A. For simplicity, only one double stranded target nucleic acid is shown.

FIG. 3 shows a general block diagram of one embodiment of a general illustrative method for single-cell combinatorial indexing according to the present disclosure.

FIG. 4 shows a general block diagram of one embodiment of a general illustrative method for single-cell combinatorial indexing according to the present disclosure.

FIG. 5 shows a schematic drawing of a method for single-cell combinatorial indexing as generally illustrated in the method of FIG. 1, FIG. 3, or FIG. 4. For simplicity, only one double stranded target nucleic acid is shown.

FIG. 6 shows a general block diagram of one embodiment of a general illustrative method for metagenomic analysis with single-cell combinatorial indexing according to the present disclosure.

FIG. 7 shows a schematic drawing of one embodiment of a general illustrative method for producing a sequencing library with contiguous indexes according to the present disclosure.

FIG. 8 shows a schematic drawing of one embodiment of a general illustrative method for coupling enrichment with targeted amplification according to the present disclosure.

FIG. 9 shows a schematic of sci-ATAC-seq3. Nuclei of 1.6. million cells from 59 fetal samples were tagmented with Tn5 transposase in bulk. The first two rounds of indexing were achieved by successive ligation to each end of the Tn5 transposase complex, and the third round by PCR. The first round of indexing was used as a sample index.

FIG. 10 shows the structure of amplicons resulting from sci-ATAC-seq3 described in Example 1.

FIG. 11 shows the project workflow described in Example 2.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION OF ILLUSTRATIVE EMBODIMENTS

The methods provided herein can be used to produce sequencing libraries from a plurality of single cells. Essentially any single-nuclei or single-cell library preparation method or sequencing method can be used including, but not limited to, single-cell combinatorial indexing methods such as single-nuclei sequencing of transposon accessible chromatin (sci-ATAC, U.S. Pat. No. 10,059,989), whole genome sequencing of single-nuclei (U.S. Pat. Appl. Pub. No. US 2018/0023119), single-nuclei transcriptome sequencing (U.S. Prov. Pat. App. No. 62/680,259 and Gunderson et al. (WO2016/130704)), sci-HiC (Ramani et al., Nature Methods, 2017, 14:263-266), DRUG-seq (Ye et al., Nature Commun., 9, article number 4307), or any combination of analytes from DNA and proteins, for example sci-CAR (Cao et al., Science, 2018, 361(6409):1380-1385) and RNA and proteins, for example CITE-seq (Stoeckius et al., 2017, Nature Methods. 14 (9): 865-868). In one embodiment, cell atlas experiments can be conducted with the readout restricted to chromatin accessible DNA, whole cell transcriptomes, a limited number of mRNAs that are highly informative, or a combination thereof.

Providing Isolated Nuclei or Cells

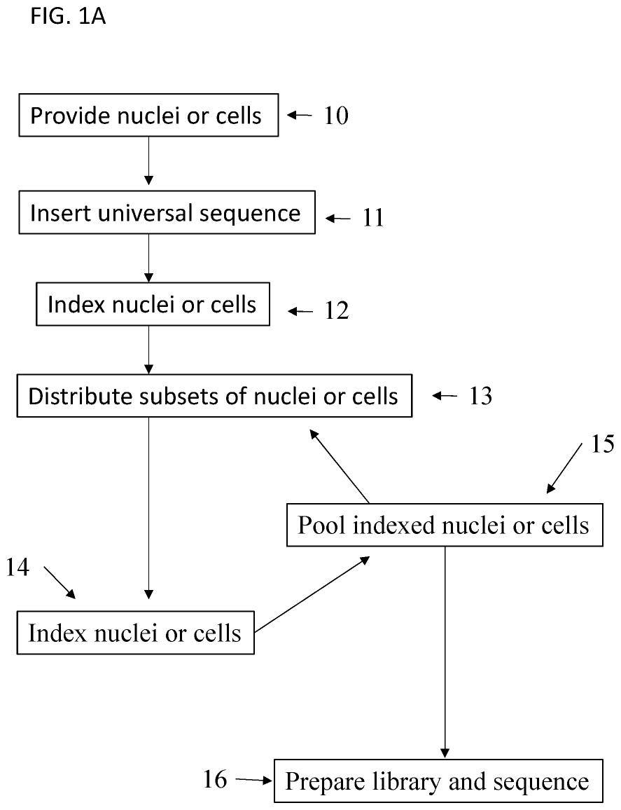

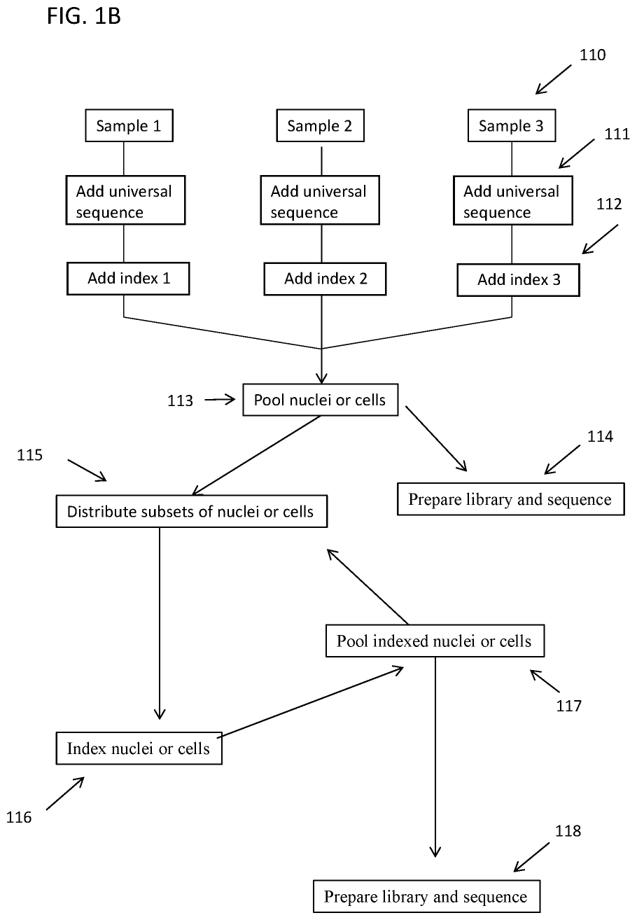

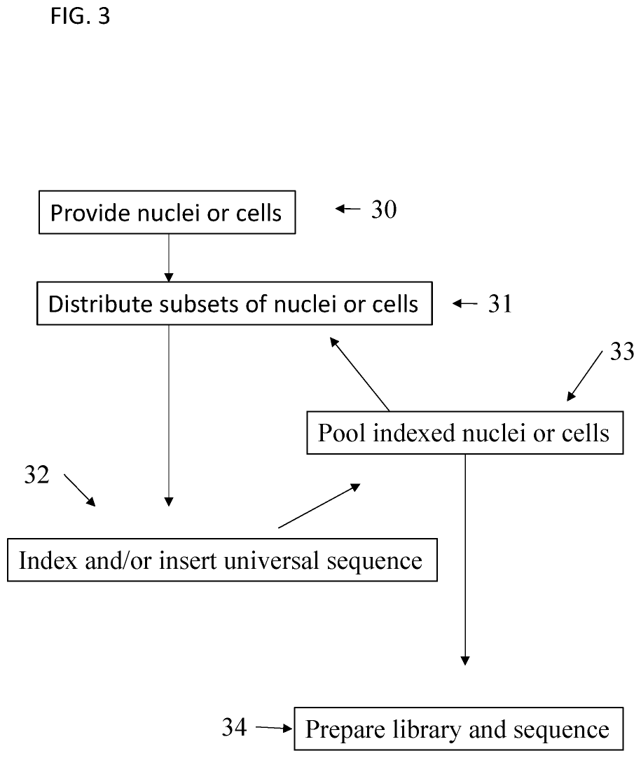

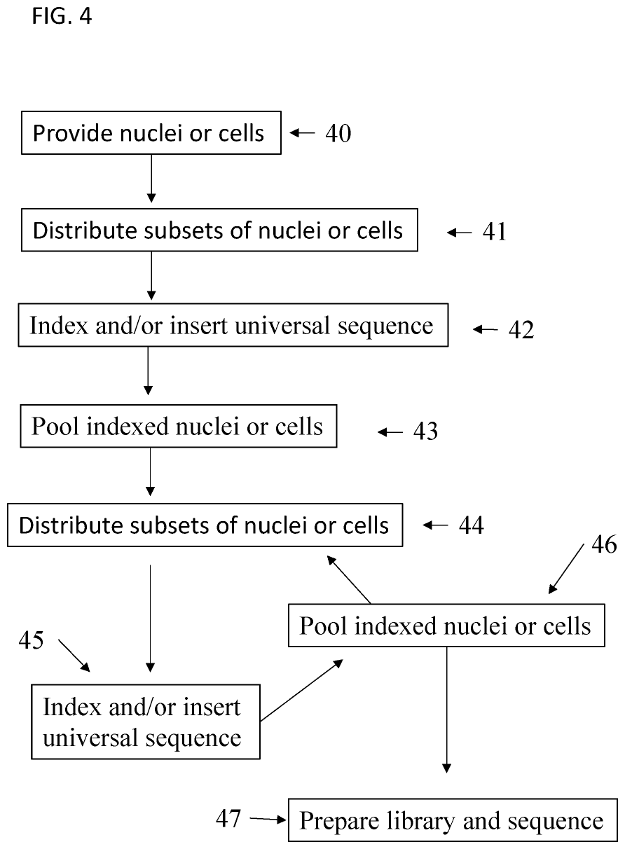

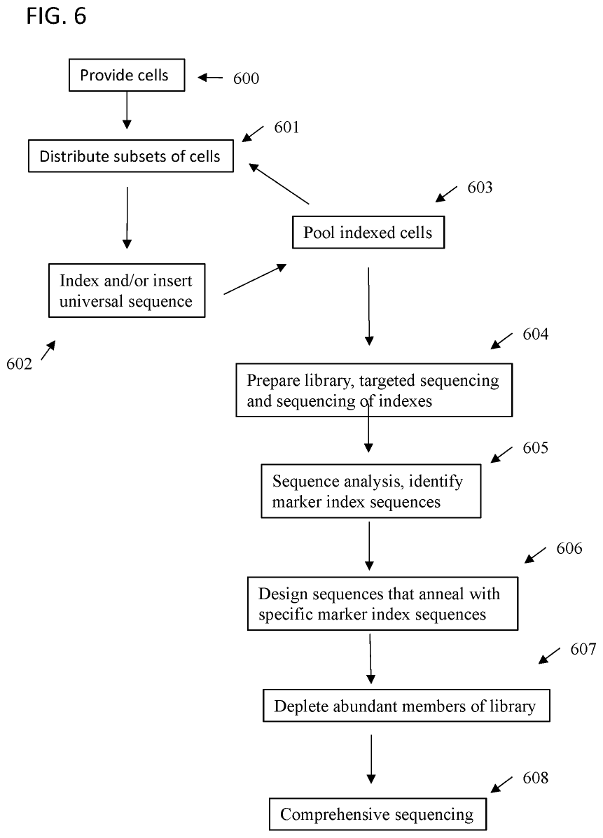

In one embodiment, the method provided herein can include providing the cells or isolated nuclei from a plurality of cells (e.g., FIG. 1A, block 10, FIG. 3, block 30, FIG. 4, block 40, FIG. 6, block 600). The cells can be from any organism(s), and from any cell type or any tissue of the organism(s). In one embodiment, the cells can be from a biopsy, such as tissue or liquid biopsy. In one embodiment, the cells can be embryonic cells, e.g., cells obtained from an embryo. In one embodiment, the cells or nuclei can be from cancer or a diseased tissue. In one embodiment, the cells or nuclei can be immune cells, such as T cells or B cells. In one embodiment, the cells can be a variety of different cell types obtained from a single organism. In one embodiment, the variety of different cell types obtained from a single organism can include microbial cells, including prokaryotic and/or eukaryotic cells. In one embodiment, cells from different sources, e.g., different organisms and/or different tissues, are not combined at this stage. In one embodiment, cells from different sources, e.g., different organisms and/or different tissues, are combined at this stage.

In one embodiment, the plurality of cells can be a subset of a larger population of cells. The subset can be separated from other cells based on differences in, for instance, size, morphology, or presence of an identifiable molecule like a protein or glycan on the cell's surface. Methods for sorting cells are known in the art and include fluorescent activated cell sorting, magnetic activated cell sorting, and microfluidic cell sorting.

The method can further include dissociating cells, and/or isolating the nuclei. In one embodiment, conditions are used that maintain the chromatin present in the nuclei. In one embodiment, the nucleosomes present in nuclei are depleted. Methods for nucleosome-depletion are known to the skilled person (US Published Patent Application 2018/002311).

Many different single-cell library preparation methods are known in the art. (Hwang et al. Experimental & Molecular Medicine, vol. 50, Article number: 96 (2018), including, but not limited to, Drop-seq, Seq-well, and single-cell combinatorial indexing (“sci-”) methods. Companies providing single cell products and related technologies include, but are not limited to, 10× Genomics, Takara biosciences, BD biosciences, Biorad, 1cellbio, IsoPlexis, CellSee, NanoCellect, and Dolomite Bio. Sci-seq is a methodological framework that employs split-pool barcoding to uniquely label the nucleic acid contents of large numbers of single cells or nuclei. Typically, number of nuclei or cells can be at least two. The upper limit is dependent on the practical limitations of equipment (e.g., multi-well plates, number of indexes) used in other steps of the method as described herein. The number of nuclei or cells that can be used is not intended to be limiting and can number in the billions. For instance, in one embodiment the number of nuclei or cells can be no greater than 1,000,000,000, no greater than 100,000,000, no greater than 10,000,000, no greater than 1,000,000, no greater than 100,000, no greater than 10,000, no greater than 1,000, no greater than 500, or no greater than 50. In one embodiment the number of nuclei or cells can be at least 50, at least 500, at least 1,000, at least 10,000, at least 100,000, at least 1,000,000, at least 10,000,000, at least 100,000,000, or at least 1,000,000,000.

In those embodiments using isolated nuclei, the nuclei can be obtained by extraction and fixation. Optionally and preferably, the method of obtaining isolated nuclei does not include enzymatic treatment.

In one embodiment, nuclei are isolated from individual cells that are adherent or in suspension. Methods for isolating nuclei from individual cells are known to the person of ordinary skill in the art. Nuclei are typically isolated from cells present in a tissue. The method for obtaining isolated nuclei typically includes preparing the tissue, isolating the nuclei from the prepared tissue, and then fixing the nuclei. In one embodiment all steps are done on ice.

In one embodiment, tissue preparation includes snap freezing the tissue in liquid nitrogen, and then reducing the size of the tissue to pieces of 1 mm or less in diameter. Tissue can be reduced in size by subjecting the tissue to either mincing or a blunt force. Mincing can be accomplished with a blade to cut the tissue to small pieces. Applying a blunt force can be accomplished by smashing the tissue with a hammer or similar object, and the resulting composition of smashed tissue is referred to as a powder.

Nuclei isolation can be accomplished by incubating the pieces or powder in cell lysis buffer for at least 1 to 20 minutes, such as 5, 10, or 15 minutes. Useful buffers are those that promote cell lysis but retain nuclei integrity. An example of a cell lysis buffer includes 10 mM Tris-HCl, pH 7.4, 10 mM NaCl, 3 mM MgCl2, 0.1% IGEPAL CA-630, 1% SUPERase In RNAse Inhibitor (20 U/μL, Ambion) and 1% BSA (20 mg/ml, NEB). Standard nuclei isolation methods often use one or more exogenous compounds, such as exogenous enzymes, to aid in the isolation. Examples of useful enzymes that can be present in a cell lysis buffer include, but are not limited to, protease inhibitors, lysozyme, Proteinase K, surfactants, lysostaphin, zymolase, cellulose, protease or glycanase, and the like (Islam et al. Micromachines (Basel), 2017, 8(3):83; www.sigmaaldrich.com/life-science/biochemicals/biochemical-products.html?TablePage=14573107). In one embodiment, one or more exogenous enzymes are not present in a cell lysis buffer useful in the method described herein. For instance, an exogenous enzyme (i) is not added to the cells prior to mixing of cells and lysis buffer, (ii) is not present in a cell lysis buffer before it is mixed with cells, (iii) is not added to the mixture of cells and cell lysis buffer, or a combination thereof. The skilled person will recognize these levels of the components can be altered somewhat without reducing the usefulness of the cell lysis buffer for isolating nuclei. The extracted nuclei are then purified by one of more rounds of washing with a nuclei buffer. An example of a nuclei buffer includes 10 mM Tris-HCl, pH 7.4, 10 mM NaCl, 3 mM MgCl2, 1% SUPERase In RNAse Inhibitor (20 U/μL, Ambion) and 1% BSA (20 mg/ml, NEB). Like a cell lysis buffer, exogenous enzymes can also be absent from a nuclei buffer used in a method of the present disclosure. The skilled person will recognize these levels of the components can be altered somewhat without reducing the usefulness of the nuclei buffer for isolating nuclei. The skilled person will recognize that BSA and/or surfactants can be useful in the buffers used for the isolation of nuclei.

Isolated nuclei can be fixed by exposure to a cross-linking agent. Useful examples of cross-linking agents include, but are not limited to, paraformaldehyde and formaldehyde. The paraformaldehyde can be at a concentration of 1% to 8%, such as 4%. The formaldehyde can be at a concentration of 30% to 45%, such as 37%. Treatment of nuclei with a cross-linking agent can include adding the agent to a suspension of nuclei and incubating at 0° C. Other methods of fixation include, but are not limited to, methanol fixation. Optionally and preferably, fixation is followed by washing in a nuclei buffer.

Isolated fixed nuclei can be used immediately or aliquoted and flash frozen in liquid nitrogen for later use. When prepared for use after freezing, thawed nuclei can be permeabilized, for instance with 0.2% triton X-100 for 3 minutes on ice, and briefly sonicated to reduce nuclei clumping.

Conventional tissue nuclei extraction techniques normally incubate tissues with tissue specific enzyme (e.g., trypsin) at high temperature (e.g., 37° C.) for 30 minutes to several hours, and then lyse the cells with cell lysis buffer for nuclei extraction. The nuclei isolation method described herein has several advantages: (1) No artificial enzymes are introduced, and all steps are done on ice. This reduces potential perturbation to cell states (e.g., chromatin organization or transcriptome state). (2) The new method has been validated across most tissue types including brain, lung, kidney, spleen, heart, cerebellum, and disease samples such as tumor tissues. Compared with conventional tissue nuclei extraction techniques that use different enzymes for different tissue types, the new technique can potentially reduce bias when comparing cell states from different tissues. (3) The new method also reduces cost and increases efficiency by removing the enzyme treatment step. (4) Compared with other nuclei extraction techniques (e.g., Dounce tissue grinder), the new technique is more robust for different tissue types (e.g., the Dounce method needs optimizing Dounce cycles for different tissues), and enables processing large pieces of samples in high throughput (e.g., the Dounce method is limited to the size of the grinder).

Optionally, the isolated nuclei can be nucleosome-free or can be subjected to conditions that deplete the nuclei of nucleosomes, generating nucleosome-depleted nuclei.

Insertion of Universal Sequences

The method provided herein includes inserting one or more universal sequences into the nucleic acids present in the nuclei or cells. In one embodiment, incorporation of one or more universal sequences occurs before distribution of subsets (FIG. 1A, block 11, FIG. 1B, block 110), and in other embodiments incorporation of one or more universal sequences occurs after distribution of subsets (FIG. 3, block 32, FIG. 4, block 42, block 45). In some embodiments, an index can also be incorporated with a universal sequence, or can be associated with cells or nuclei as an optional step that is separate from the insertion of one or more universal sequences. The optional indexing of nuclei or cells can occur before or after (FIG. 1A, block 12) the insertion of a universal sequence. In one embodiment, an index is added to a sample before distributing subsets of nuclei or cells (FIG. 1A, block 13). In some embodiments, an index is added to multiple samples before distributing subsets of nuclei or cells (FIG. 1A, block 13).

In one embodiment, a transposome complex is used. A transposome complex is a transposase bound to a transposase recognition site and can insert the transposase recognition site into a target nucleic acid within a nucleus in a process sometimes termed “tagmentation.” In some such insertion events, one strand of the transposase recognition site may be transferred into the target nucleic acid. Such a strand is referred to as a “transferred strand.” In one embodiment, a transposome complex includes a dimeric transposase having two subunits, and two non-contiguous transposon sequences. In another embodiment, a transposase includes a dimeric transposase having two subunits, and a contiguous transposon sequence. In one embodiment, the 5′ end of one or both strands of the transposase recognition site may be phosphorylated.

Some embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35: 785, 1983; Savilahti, H, et al., EMBO J., 14: 4893, 1995). Tn5 Mosaic End (ME) sequences can also be used by a skilled artisan.

More examples of transposition systems that can be used with certain embodiments of the compositions and methods provided herein include Staphylococcus aureus Tn552 (Colegio et al., J. Bacteriol., 183: 2384-8, 2001; Kirby C et al., Mol. Microbiol., 43: 173-86, 2002), Ty1 (Devine & Boeke, Nucleic Acids Res., 22: 3765-72, 1994 and International Publication WO 95/23875), Transposon Tn7 (Craig, N L, Science. 271: 1512, 1996; Craig, N L, Review in: Curr Top Microbiol Immunol., 204:27-48, 1996), Tn/O and IS10 (Kleckner N, et al., Curr Top Microbiol Immunol., 204:49-82, 1996), Mariner transposase (Lampe D J, et al., EMBO J., 15: 5470-9, 1996), Tc1 (Plasterk R H, Curr. Topics Microbiol. Immunol., 204: 125-43, 1996), P Element (Gloor, G B, Methods Mol. Biol., 260: 97-114, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204: 1-26, 1996), retroviruses (Brown, et al., Proc Natl Acad Sci USA, 86:2525-9, 1989), and retrotransposon of yeast (Boeke & Corces, Annu Rev Microbiol. 43:403-34, 1989). More examples include IS5, Tn10, Tn903, IS911, and engineered versions of transposase family enzymes (Zhang et al., (2009) PLoS Genet. 5:e1000689. Epub 2009 Oct. 16; Wilson C. et al (2007) J. Microbiol. Methods 71:332-5).

Other examples of integrases that may be used with the methods and compositions provided herein include retroviral integrases and integrase recognition sequences for such retroviral integrases, such as integrases from HIV-1, HIV-2, SIV, PFV-1, RSV.

Transposon sequences useful with the methods and compositions described herein are provided in U.S. Patent Application Pub. No. 2012/0208705, U.S. Patent Application Pub. No. 2012/0208724 and Int. Patent Application Pub. No. WO 2012/061832. In some embodiments, a transposon sequence includes a first transposase recognition site and a second transposase recognition site.

Some transposome complexes useful herein include a transposase having two transposon sequences. In some such embodiments, the two transposon sequences are not linked to one another, in other words, the transposon sequences are non-contiguous with one another. Examples of such transposomes are known in the art (see, for instance, U.S. Patent Application Pub. No. 2010/0120098).

In one embodiment, tagmentation is used to produce target nucleic acids that include different universal sequences at each end (e.g., a universal primer binding site such as A14 at one end and a universal primer binding site such as B15 at the other end). This can be accomplished by using two types of transposome complexes, where each transposome complex includes a different nucleotide sequence that is part of the transferred strand. The universal sequence can serve multiple purposes. For instance and without intending to be limiting, it can serve as a complementary sequence for hybridization in a subsequent amplification step for addition of another nucleotide sequence, e.g., an index, it can serve as a site to which a universal primer (e.g., a sequencing primer for read 1 or read 2) anneals for sequencing, or it can serve as a “landing pad” in a subsequent step to anneal a nucleotide sequence that can be used as a primer for addition of another nucleotide sequence, such as an index, to a target nucleic acid.

In some embodiments, a transposome complex includes a transposon sequence nucleic acid that binds two transposase subunits to form a “looped complex” or a “looped transposome.” In one example, a transposome includes a dimeric transposase and a transposon sequence. Looped complexes can ensure that transposons are inserted into target DNA while maintaining ordering information of the original target DNA and without fragmenting the target DNA. As will be appreciated, looped structures may insert desired nucleic acid sequences, such as universal sequences, into a target nucleic acid, while maintaining physical connectivity of the target nucleic acid. In some embodiments, the transposon sequence of a looped transposome complex can include a fragmentation site such that the transposon sequence can be fragmented to create a transposome complex comprising two transposon sequences. Such transposome complexes are useful to ensuring that neighboring target DNA fragments, in which the transposons insert, receive barcode combinations that can be unambiguously assembled at a later stage of the assay. In one embodiment, index combinations are added after insertion of one or more universal sequences into a target nucleic acid.

In one embodiment, fragmenting nucleic acids is accomplished by using a fragmentation site present in the nucleic acids. Typically, fragmentation sites are introduced into target nucleic acids by using a transposome complex. In one embodiment, after nucleic acids are fragmented the transposase remains attached to the nucleic acid fragments, such that nucleic acid fragments derived from the same genomic DNA molecule remain physically linked (Adey et al., 2014, Genome Res., 24:2041-2049, Amini S. et al. (2014) Nat Genet 46: 1343-1349). For instance, a looped transposome complex can include a fragmentation site. A fragmentation site can be used to cleave the physical association, but not the informational association between index sequences that have been incorporated into a target nucleic acid. Cleavage may be by biochemical, chemical or other means. In some embodiments, a fragmentation site can include a nucleotide or nucleotide sequence that may be fragmented by various means. Examples of fragmentation sites include, but are not limited to, a restriction endonuclease site, at least one ribonucleotide cleavable with an RNAse, nucleotide analogues cleavable in the presence of a certain chemical agent, a diol linkage cleavable by treatment with periodate, a disulfide group cleavable with a chemical reducing agent, a cleavable moiety that may be subject to photochemical cleavage, and a peptide cleavable by a peptidase enzyme or other suitable means (see, for instance, U.S. Patent Application Pub. No. 2012/0208705, U.S. Patent Application Pub. No. 2012/0208724 and WO 2012/061832). In one embodiment, a transposase remains attached to the nucleic acid fragments and maintains the physical linkage between nucleic acid fragments derived from the same genomic DNA molecule until removal by use of appropriate conditions, such as the addition of a protein denaturing agent, e.g., SDS, or a chelating agent, e.g., EDTA. This type of approach permits derivation of contiguity information by means of capturing contiguously-linked, transposed, target nucleic acid (US Pat. Application No. 2019/0040382). Contiguity information can be preserved by the use of transposase to maintain the association of template nucleic acid fragments adjacent in the target nucleic acid.

As an alternative to transposition, target nucleic acids can be obtained by fragmentation. Fragmentation of primary nucleic acids from a sample can be accomplished in a non-ordered fashion by enzymatic, chemical, or mechanical methods, and adapters are then added to the ends of the fragments. Examples of enzymatic fragmentation include CRISPR and Talen-like enzymes, and enzymes that unwind DNA (e.g. Helicases) that can make single stranded regions to which DNA fragments can hybridize and initiate extension or amplification. For example, helicase-based amplification can be used (Vincent et al., 2004, EMBO Rep., 5(8):795-800). In one embodiment, the extension or amplification is initiated with a random primer. Examples of mechanical fragmentation include nebulization or sonication.

Fragmentation of primary nucleic acids by mechanical means results in fragments with a heterogeneous mix of blunt and 3′- and 5′-overhanging ends. It is therefore desirable to repair the fragment ends using methods known in the art to generate ends that are optimal for addition of adapters, for example, into blunt sites. In a particular embodiment, the fragment ends of the population of nucleic acids are blunt ended. More particularly, the fragment ends are blunt ended and phosphorylated. The phosphate moiety can be introduced via enzymatic treatment, for example, using polynucleotide kinase.

In one embodiment, the fragmented nucleic acids are prepared with overhanging nucleotides. For example, single overhanging nucleotides can be added by the activity of certain types of DNA polymerase such as Taq polymerase or Klenow exo minus polymerase which has a non-template-dependent terminal transferase activity that adds a single deoxynucleotide, for example, the nucleotide ‘A’ to the 3′ ends of a DNA molecule. Such enzymes can be used to add a single nucleotide ‘A’ to the blunt ended 3′ terminus of each strand of double-stranded nucleic acid fragments. Thus, an ‘A’ could be added to the 3′ terminus of each end repaired strand of the double-stranded target fragments by reaction with Taq or Klenow exo minus polymerase, while the adapter could be a T-construct with a compatible ‘T’ overhang present on the 3′ terminus of each region of double stranded nucleic acid of the universal adapter. In one example, terminal deoxynucleotidyl transferase (TdT) can be used to add multiple ‘T’ nucleotides (Swift Biosciences, Ann Arbor, Mich.). This type of end modification also prevents self-ligation of both vector and target such that there is a bias towards formation of the target nucleic acids having the same adapter at each end.

The primary nucleic acid can be DNA, RNA, or DNA/RNA hybrids. In those embodiments where the primary nucleic acid is RNA, incorporating one or more universal sequences into the nucleic acids present in the nuclei or cells typically includes the conversion of RNA into DNA. Various methods can be used, and in some embodiments include the routine methods used to produce cDNA. For instance, a primer with a poly-T sequence at the 3′ end and an adapter upstream of the poly-T sequence can be annealed to mRNA molecules and extended using a reverse transcriptase. This results in a one-step conversion of mRNA to DNA and optionally a universal sequence to the 3′ end. In one embodiment, the primer can also include one or more index sequences. In one embodiment, a random primer is used.

A non-coding RNA can also be converted into DNA and optionally modified to include a universal sequence using various methods. For example, an adapter can be added using a first primer that includes a random sequence and a template-switch primer, where either primer can include a universal sequence adapter. A reverse transcriptase having a terminal transferase activity to result in addition of non-template nucleotides to the 3′ end of the synthesized strand can be used, and the template-switch primer includes nucleotides that anneal with the non-template nucleotides added by the reverse transcriptase. An example of a useful reverse transcriptase enzyme is a Moloney murine leukemia virus reverse transcriptase. In a particular embodiment, the SMARTer™ reagent available from Takara Bio USA, Inc. (Cat. 634926) is used for the use of template-switching to add a universal sequence to non-coding RNA, and mRNA if desired. Optionally, a template-switch primer can be used with mRNA in conjunction with a primer with a poly-T sequence to result in adding a universal sequence to both ends of a DNA target nucleic acid produced from RNA.

Distributing Subsets

The method provided herein includes distributing subsets of the isolated nuclei or cells into a plurality of compartments (FIG. 1A, block 13, FIG. 1B, block 115, FIG. 3, block 31, FIG. 4, block 41, block 44). The method can include multiple distribution steps, where a population of isolated nuclei or cells (also referred to herein as a pool) is split into subsets. Typically, subsets of isolated nuclei or cells, e.g., subsets present in a plurality of compartments, are indexed with compartment specific indexes and then pooled. Accordingly, the method typically includes at least one “split and pool” step of taking pooled isolated nuclei or cells, distributing them, and adding a compartment specific index, where the number of “split and pool” steps can depend on the number of different indexes that are added to the target nucleic acids. Each initial subset of nuclei or cells prior to indexing can be unique from other subsets. For example, each first subset can be from a unique sample such as a unique organism or a unique tissue. After indexing, the subsets can be pooled, split into subsets, indexed, and pooled again as needed until a sufficient number of indexes are added to the target nucleic acids. This process assigns unique index or index combinations to each single cell or nucleus and results in combinatorial indexing, which is described herein. After indexing is complete, e.g., after one, two, three, or more indexes are added, the isolated nuclei or cells can be lysed. In some embodiments, adding an index and lysing can occur simultaneously.

The number of nuclei or cells present in a subset, and therefore in each compartment, can be at least 1. In one embodiment, the number of nuclei or cells present in a subset is no greater than 100,000,000, no greater than 10,000,000, no greater than 1,000,000, no greater than 100,000, no greater than 10,000, no greater than 4,000, no greater than 3,000, no greater than 2,000, or no greater than 1,000, no greater than 500, or no greater than 50. In one embodiment, the number of nuclei or cells present in a subset can be 1 to 1,000, 1,000 to 10,000, 10,000 to 100,000, 100,000 to 1,000,000, 1,000,000 to 10,000,000, or 10,000,000 to 100,000,000. In one embodiment, the number of nuclei or cells present in each subset is approximately equal. The number of nuclei or cells present in a subset, and therefor in each compartment, is based in part on the desire to reduce index collisions, which is the presence of two nuclei or cells having the same index combination ending up in the same compartment in this step of the method. Methods for distributing nuclei or cells into subsets are known to the person skilled in the art and are routine. While fluorescence-activated cell sorting (FACS) cytometry can be used, use of simple dilution is preferred in some embodiments. In one embodiment, FACS cytometry is not used. Optionally, nuclei of different ploidies can be gated and enriched by staining, e.g., DAPI (4′,6-diamidino-2-phenylindole) staining. Staining can also be used to discriminate single cells from doublets during sorting.

The number of compartments in the distribution steps (and subsequent addition of an index) can depend on the format used. For instance, the number of compartments can be from 2 to 96 compartments (when a 96-well plate is used), from 2 to 384 compartments (when a 384-well plate is used), or from 2 to 1536 compartments (when a 1536-well plate is used). In one embodiment, multiple plates can be used. Examples of compartments include, but are not limited to, a well, a droplet, and a microfluidic compartment. In one embodiment, each compartment can be a droplet. When the type of compartment used is a droplet that contains two or more nuclei or cells, any number of droplets can be used, such as at least 10,000, at least 100,000, at least 1,000,000, or at least 10,000,000 droplets. Subsets of isolated nuclei or cells are typically indexed in compartments before pooling.

Combinatorial Indexing

The method provided herein includes adding a compartment specific index to the nuclei or cells present in a sample (FIG. 1B, block 112) or to subsets of the isolated nuclei or cells distributed to different compartments (e.g., FIG. 1A, block 14, FIG. 3, block 32, FIG. 4, block 42 and 45, FIG. 6, block 601). In some embodiments, a universal sequence can also be incorporated with an index. An index sequence, also referred to as a tag or barcode, is useful as a marker characteristic of the compartment in which a particular nucleic acid was present. Accordingly, in some embodiments an index is a nucleic acid sequence tag which is attached to each of the target nucleic acids present in a particular compartment, the presence of which is indicative of, or is used to identify, the compartment in which a population of isolated nuclei or cells were present at a particular stage of the method.

In one embodiment, multiple indexes are added. The incorporation of each index occurs in one round of split and pool indexing. One, two, three, or more rounds of split and pool barcoding results in single, dual, triple, or multiple (e.g., four or more) indexed target nucleic acids.

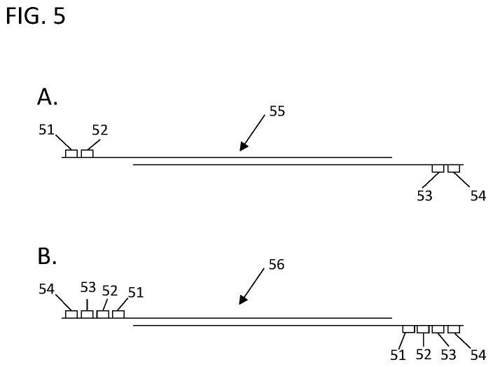

Indexes can be added to one or both ends of a target nucleic acid. For instance, modified target nucleic acids having two or more indexes can include different indexes at each end, an example of which is shown in FIG. 5A. In FIG. 5A, a target nucleic acid 55 is modified to include four distinct indexes, two indexes (51 and 52) at one end and two indexes (53 and 54) at the other end. In other embodiments, a modified target nucleic acid can include the indexes grouped together at one end or at both ends, an example of which is shown in FIG. 5B. In FIG. 5B, a target nucleic acid 56 is modified to include four distinct indexes (51, 52, 53, and 54) at each end. A set of indexes that are present on one end of a target nucleic acid can be referred to as a “contiguous index.” In one embodiment, contiguous indexes have no nucleotides between each of the indexes. In other embodiments there can be 1, 2, 3, 4, or more nucleotides located between one or more of the indexes of a contiguous index. As described herein, a contiguous index can be useful in identifying members of a library having a specific set of indexes. For instance, a contiguous index can facilitate the enrichment of library members that originate from the same cell.

An index sequence can be any suitable number of nucleotides in length, e.g., 1, 2, 3, 4, 5, 6, 7, 8, 9, 10, 11, 12, 13, 14, 15, 16, 17, 18, 19, 20, or more. A four nucleotide tag gives a possibility of multiplexing 256 samples on the same array, and a six base tag enables 4096 samples to be processed on the same array.

In one embodiment, an index is added after a universal sequence is incorporated into DNA nucleic acids of nuclei or cells by, for instance, a transposome complex. The incorporation of an index sequence can use a process that includes one, two, or more steps, using essentially any combination of ligation, extension, hybridization, adsorption, specific or non-specific interactions of a primer, or amplification. In one embodiment, an index is added during cDNA synthesis. In one embodiment, the index is added through tagmentation. The nucleotide sequence that is added to one or both ends of the target nucleic acids can also include other useful sequences such as one or more universal sequences and/or unique molecular identifiers.

Various methods can be used for the addition of an index to a nucleic acid that includes a universal sequence, and how an index is added is not intended to be limiting. In one embodiment, the target nucleic acids have a different universal sequence at each end (e.g., A14 at one end and B15 at the other end), and the skilled person will recognize that specific sequences can be added to one or both ends of a target nucleic acid. The universal sequences added by the transposome complex can be used as, for instance, a “landing pad” in a subsequent step to anneal a nucleotide sequence that can be used as a primer for addition of another nucleotide sequence, such as another index and/or another universal sequence, to a target nucleic acid. For instance, in one embodiment the incorporation of an index sequence includes ligating a primer to one or both ends of the nucleic acids. The ligation of a primer can be aided by the presence of the universal sequence at each end of the target nucleic acids. An example of a primer is a hairpin ligation duplex. The ligation duplex can be ligated to one end or preferably both ends of target nucleic acids.

In one embodiment, blunt-ended ligation can be used. In another embodiment, the target nucleic acids are prepared with single overhanging nucleotides by, for example, activity of certain types of DNA polymerase such as Taq polymerase or Klenow exo minus polymerase which has a non-template-dependent terminal transferase activity that adds one or more deoxynucleotides, for example, deoxyadenosine (A) to the 3′ ends of the target nucleic acids. In some cases, the overhanging nucleotide is more than one base. Such enzymes can be used to add a single nucleotide ‘A’ to the blunt ended 3′ terminus of each strand of the target nucleic acids. Thus, an ‘A’ could be added to the 3′ terminus of each strand of the double-stranded target fragments by reaction with Taq or Klenow exo minus polymerase, while the additional sequences to be added to each end of the target nucleic acid can include a compatible ‘T’ overhang present on the 3′ terminus of each region of double stranded nucleic acid to be added. This end modification also prevents self-ligation of the nucleic acids such that there is a bias towards formation of the indexed target nucleic acids flanked by the sequences that are added in this embodiment.

In one embodiment, incorporation of an index is by an exponential amplification reaction, such as a PCR. The universal sequences present at ends of target nucleic acids can be used for the annealing of a sequence which can serve as primers and be extended in an amplification reaction.

An index and other useful sequences can be added in a single step or in multiple steps. For example, an index and any other useful sequences can be added by a ligation or extension, or a two-step method can be used that includes, for instance, ligating a universal sequence and then an amplification to further modify the universal sequence to include an index and any other useful sequences.

In one embodiment, the addition of sequences during the indexing steps add universal sequences useful in the immobilizing and/or sequencing the target nucleic acids. In another embodiment, the indexed target nucleic acids can be further processed to add universal sequences useful in immobilizing and sequencing the target nucleic acids. The skilled person will recognize that in embodiments where the compartment is a droplet sequences for immobilizing nucleic acid fragments are optional. In one embodiment, the incorporation of universal sequences useful in immobilizing and sequencing the fragments includes ligating identical universal adapters (also referred to as ‘mismatched adaptors,’ the general features of which are described in Gormley et al., U.S. Pat. No. 7,741,463, and Bignell et al., U.S. Pat. No. 8,053,192,) to the 5′ and 3′ ends of the indexed nucleic acid fragments. In one embodiment, the universal adaptor includes all sequences necessary for sequencing, including sequences for immobilizing the indexed nucleic acid fragments on an array.

The resulting indexed fragments collectively provide a library of nucleic acids that can be immobilized and then sequenced. The term library, also referred to herein as a sequencing library, refers to the collection of target nucleic acids from single nuclei or cells containing known universal sequences and various combinations of indexes at their 3′ and 5′ ends. The library includes nucleic acids from, for instance, the accessible DNA, the whole genome, or the whole transcriptome, nucleic acids indicative of a specific protein, or a combination thereof, and can be used to perform sequencing.

The indexed nucleic acid fragments can be subjected to conditions that select for a predetermined size range, such as from 150 to 400 nucleotides in length, such as from 150 to 300 nucleotides. The resulting indexed nucleic acid fragments are pooled, and optionally can be subjected to a clean-up process to enhance the purity to the DNA molecules by removing at least a portion of unincorporated universal adapters or primers. Any suitable clean-up process may be used, such as electrophoresis, size exclusion chromatography, or the like. In some embodiments, solid phase reversible immobilization paramagnetic beads may be employed to separate the desired DNA molecules from unattached universal adapters or primers, and to select nucleic acids based on size. Solid phase reversible immobilization paramagnetic beads are commercially available from Beckman Coulter (Agencourt AMPure XP), Thermofisher (MagJet), Omega Biotek (Mag-Bind), Promega Beads (Promega), and Kapa Biosystems (Kapa Pure Beads).

A non-limiting illustrative embodiment of the present disclosure is shown in FIG. 1A. In this embodiment, the method includes providing a plurality of nuclei or cells (FIG. 1A, block 10). The plurality of nuclei or cells can be from a sample or from a plurality of samples. The method further includes incorporation of one or more universal sequences into nucleic acids present in the nuclei or cells (FIG. 1A, block 11). Optionally, the method can also include associating an index to the nuclei or cells (e.g., nuclear or cellular hashing, see WO 2020/180778), and in one embodiment the associating can be addition of an index to the nucleic acids (FIG. 1A, block 12). In one embodiment, two different universal sequences are added to ultimately result in target nucleic acids with a different universal sequence at each end. The method further includes distributing subsets of nuclei or cells, now including universal sequences incorporated into nucleic acids located therein, and optionally, at least one index, into a plurality of compartments (FIG. 1A, block 13). The nuclei acids present in each compartment are indexed (FIG. 1A, block 14), and the nuclei or cells are then pooled (FIG. 1A, block 15). After the addition of a single index, the libraries of nucleic acids in the nuclei or cells can be further processed to prepare for sequencing (FIG. 1A, block 16); however, in some preferred embodiments addition of a second, third, or more indexes is desirable. In one embodiment, addition of each index can include a “split and pool” step with indexing occurring after the split, e.g., distributing subsets of nuclei or cells into a plurality of compartments (FIG. 1A, block 13), indexing the nuclei acids present in each compartment (FIG. 1A, block 14), and then pooling the nuclei or cells (FIG. 1A, block 15). A “split and pool” step can result in the addition of an index to only one end or to both ends of the nucleic acids present in the nuclei or cells. After addition of the last index the libraries of nucleic acids in the nuclei or cells can be pooled and further processed to prepare for sequencing (FIG. 1A, block 16), where the sequencing can be comprehensive or targeted.

Another non-limiting illustrative embodiment of the present disclosure is shown in FIG. 1B. In this embodiment, the method includes providing a plurality of samples (FIG. 1B, block 110) that are initially processed in parallel. The method further includes incorporation of one or more universal sequences into nucleic acids present in the nuclei or cells (FIG. 1B, block 111), followed by addition of an index to the nucleic acids (FIG. 1B, block 112), where the index added to each sample is unique and can be used as a sample index to identify which nucleic acids originated from a specific sample. In one embodiment, two different universal sequences are added to ultimately result in target nucleic acids with a different universal sequence at each end. The method further includes pooling the nuclei or cells (FIG. 1B, block 113). In one embodiment, after the addition of one index, the libraries of nucleic acids in the nuclei or cells can be further processed to prepare for sequencing (FIG. 1B, block 114); however, in some preferred embodiments addition of a second, third, or more indexes is desirable. In one embodiment, addition of each index can include a “split and pool” step with indexing occurring after the split, e.g., distributing subsets of nuclei or cells into a plurality of compartments (FIG. 1B, block 115), indexing the nuclei acids present in each compartment (FIG. 1B, block 116), and then pooling the nuclei or cells (FIG. 1B, block 117). A “split and pool” step can result in the addition of an index to only one end or to both ends of the nucleic acids present in the nuclei or cells. After addition of the last index the libraries of nucleic acids in the nuclei or cells can be pooled and further processed to prepare for sequencing (FIG. 1B, block 118), where the sequencing can be comprehensive or targeted.

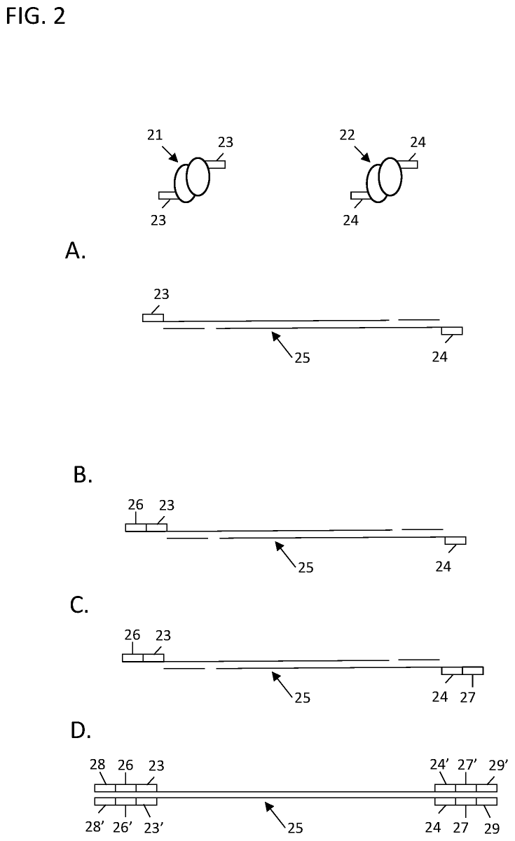

Another non-limiting illustrative embodiment of the present disclosure is shown in FIG. 2. In this embodiment, the method includes the use of tagmentation to incorporate two universal sequences into nucleic acids present in the nuclei or cells and three subsequent rounds of indexing (FIG. 2A). One transposome complex 21 includes a universal sequence 23 (e.g., A14) and another transposome complex 22 includes a universal sequence 24 (B15). The insertion of the universal sequences into the nucleic acids occurs to a plurality of nuclei or cells in bulk. FIG. 2A also shows the result of the insertion of the two universal sequences 23 and 24 into the target nucleic acid 25. The plurality of nuclei or cells are distributed to different compartments and a polynucleotide 26 including an index is added to one side of the nucleic acid 25 by ligation, using nucleotides complementary to one universal sequence (e.g., A14) (FIG. 2B). The plurality of nuclei or cells are pooled and then distributed to different compartments and a different polynucleotide 27 including a second index is added to the other side of the nucleic acid 25 by ligation, using nucleotides complementary to the other universal sequence (e.g., B15) (FIG. 2C). The plurality of nuclei or cells containing the dual-indexed nucleic acids are pooled and then distributed to different compartments, and then subjected to a PCR amplification reaction that adds a polynucleotide 28 including a third index to one side of the nucleic acid 25 and adds a polynucleotide 29 including a fourth index to one side of the nucleic acid 25 (FIG. 2D). After addition of the last index the libraries of nucleic acids in the nuclei or cells can be pooled and further processed to prepare for sequencing, where the sequencing can be comprehensive or targeted.

Yet another non-limiting illustrative embodiment of the present disclosure is shown in FIG. 3. In this embodiment, the method includes providing a plurality of nuclei or cells (FIG. 3, block 30). The method further includes distributing subsets of nuclei or cells into a plurality of compartments (FIG. 3, block 31). The nucleic acids present in the nuclei or cells of each compartment are modified by the incorporation of an index and/or a universal sequence (FIG. 3, block 32). In an alternative embodiment, the nucleic acids present in the nuclei or cells of each compartment are modified by the incorporation of the same universal sequence (e.g., tagmentation using a transposon with the same universal sequence), followed by addition of a compartment-specific index. The nuclei or cells are then pooled (FIG. 3, block 33). After the addition of an index and/or a universal sequence, the libraries of nucleic acids in the nuclei or cells can be further processed to prepare for sequencing (FIG. 3, block 34); however, in some preferred embodiments addition of a second, third, or more indexes is desirable. Optionally, universal sequences can also be added. Addition of each index can include a “split and pool” step with indexing occurring after the split, e.g., distributing subsets of nuclei or cells into a plurality of compartments (FIG. 3, block 31), indexing the nuclei acids present in each compartment (FIG. 3, block 32), and then pooling the nuclei or cells (FIG. 3, block 33). A “split and pool” step can result in the addition of an index to only one end or to both ends of the nucleic acids present in the nuclei or cells. After addition of the last index the libraries of nucleic acids in the nuclei or cells can be pooled and further processed to prepare for sequencing (FIG. 3, block 34), where the sequencing can be comprehensive or targeted.

A further non-limiting illustrative embodiment of the present disclosure is shown in FIG. 4. In this embodiment, the method includes analysis of RNA. A plurality of nuclei or cells is provided (FIG. 4, block 40), and can be from a sample or a plurality of samples. Subsets of nuclei or cells are distributed into a plurality of compartments (FIG. 4, block 41). Optionally, before the distribution the method can also include associating an index to the nuclei or cells (e.g., nuclear or cellular hashing, see WO 2020/180778) or to the nucleic acids. The nucleic acids present in the nuclei or cells of each compartment are modified by using reverse transcriptase to insert an index and/or a universal sequence (FIG. 4, block 42), and the nuclei or cells are then pooled (FIG. 4, block 43). The method further includes distributing subsets of nuclei or cells into a plurality of compartments (FIG. 4, block 44). The nucleic acids present in the nuclei or cells of each compartment are modified by the insertion of another index and/or a universal sequence (FIG. 4, block 45), and the nuclei or cells are then pooled (FIG. 4, block 46). After the addition of an index and/or a universal sequence, the libraries of nucleic acids in the nuclei or cells can be further processed to prepare for sequencing (FIG. 4, block 47); however, in some preferred embodiments addition of a third, fourth, or more indexes is desirable. Optionally, universal sequences can also be added. Addition of each index can include a “split and pool” step with indexing occurring after the split, e.g., distributing subsets of nuclei or cells into a plurality of compartments (FIG. 4, block 44), indexing the nuclei acids present in each compartment (FIG. 4, block 45), and then pooling the nuclei or cells (FIG. 4, block 46). A “split and pool” step can result in the addition of an index to only one end or to both ends of the nucleic acids present in the nuclei or cells. After addition of the last index the libraries of nucleic acids in the nuclei or cells can be pooled and further processed to prepare for sequencing (FIG. 4, block 47), where the sequencing can be comprehensive or targeted.

Preparation of Immobilized Samples for Sequencing

Methods for attaching indexed fragments from one or more sources to a substrate are known in the art. In one embodiment, indexed fragments are enriched using a plurality of capture sequences having specificity for the indexed fragments, and the capture sequences can be immobilized on a surface of a solid substrate. For instance, capture sequences can include a first member of a binding pair, (e.g., P5′), and wherein a second member of the binding pair (P5) is immobilized on a surface of a solid substrate. Likewise, methods for amplifying immobilized indexed fragments include, but are not limited to, bridge amplification and kinetic exclusion. Methods for immobilizing and amplifying prior to sequencing are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

A pooled sample can be immobilized in preparation for sequencing. Sequencing can be performed as an array of single molecules or can be amplified prior to sequencing. The amplification can be carried out using one or more immobilized primers. The immobilized primer(s) can be, for instance, a lawn on a planar surface, or on a pool of beads. The pool of beads can be isolated into an emulsion with a single bead in each “compartment” of the emulsion. At a concentration of only one template per “compartment,” only a single template is amplified on each bead.

The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Solid phase PCR covers systems such as emulsions, wherein one primer is anchored to a bead and the other is in free solution, and colony formation in solid phase gel matrices wherein one primer is anchored to the surface, and one is in free solution.

In some embodiments, the solid support comprises a patterned surface. A “patterned surface” refers to an arrangement of different regions in or on an exposed layer of a solid support. For example, one or more of the regions can be features where one or more amplification primers are present. The features can be separated by interstitial regions where amplification primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Pat. Nos. 8,778,848, 8,778,849 and 9,079,148, and US Pub. No. 2014/0243224.

In some embodiments, the solid support includes an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface can be wells in an array of wells (e.g. microwells or nanowells) on glass, silicon, plastic or other suitable solid supports with patterned, covalently-linked gel such as poly(N-(5-azidoacetamidylpentyl)acrylamide-co-acrylamide) (PAZAM, see, for example, US Pub. No. 2013/184796, WO 2016/066586, and WO 2015/002813). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However, in many embodiments the gel need not be covalently linked to the wells. For example, in some conditions silane free acrylamide (SFA, see, for example, U.S. Pat. No. 8,563,477) which is not covalently attached to any part of the structured substrate, can be used as the gel material.

In particular embodiments, a structured substrate can be made by patterning a solid support material with wells (e.g. microwells or nanowells), coating the patterned support with a gel material (e.g. PAZAM, SFA or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Primer nucleic acids can be attached to gel material. A solution of indexed fragments can then be contacted with the polished substrate such that individual indexed fragments will seed individual wells via interactions with primers attached to the gel material; however, the target nucleic acids will not occupy the interstitial regions due to absence or inactivity of the gel material. Amplification of the indexed fragments will be confined to the wells since absence or inactivity of gel in the interstitial regions prevents outward migration of the growing nucleic acid colony. The process can be conveniently manufactured, being scalable and utilizing conventional micro- or nanofabrication methods.

Although the disclosure encompasses “solid-phase” amplification methods in which only one amplification primer is immobilized (the other primer usually being present in free solution), in one embodiment it is preferred for the solid support to be provided with both the forward and the reverse primers immobilized. In practice, there will be a ‘plurality’ of identical forward primers and/or a ‘plurality’ of identical reverse primers immobilized on the solid support, since the amplification process requires an excess of primers to sustain amplification. References herein to forward and reverse primers are to be interpreted accordingly as encompassing a ‘plurality’ of such primers unless the context indicates otherwise.

As will be appreciated by the skilled reader, any given amplification reaction requires at least one type of forward primer and at least one type of reverse primer specific for the template to be amplified. However, in certain embodiments the forward and reverse primers may include template-specific portions of identical sequence, and may have entirely identical nucleotide sequence and structure (including any non-nucleotide modifications). In other words, it is possible to carry out solid-phase amplification using only one type of primer, and such single-primer methods are encompassed within the scope of the disclosure. Other embodiments may use forward and reverse primers which contain identical template-specific sequences but which differ in some other structural features. For example, one type of primer may contain a non-nucleotide modification which is not present in the other.

In all embodiments of the disclosure, primers for solid-phase amplification are preferably immobilized by single point covalent attachment to the solid support at or near the 5′ end of the primer, leaving the template-specific portion of the primer free to anneal to its cognate template and the 3′ hydroxyl group free for primer extension. Any suitable covalent attachment means known in the art may be used for this purpose. The chosen attachment chemistry will depend on the nature of the solid support, and any derivatization or functionalization applied to it. The primer itself may include a moiety, which may be a non-nucleotide chemical modification, to facilitate attachment. In a particular embodiment, the primer may include a sulphur-containing nucleophile, such as phosphorothioate or thiophosphate, at the 5′ end. In the case of solid-supported polyacrylamide hydrogels, this nucleophile will bind to a bromoacetamide group present in the hydrogel. A more particular means of attaching primers and templates to a solid support is via 5′ phosphorothioate attachment to a hydrogel comprised of polymerized acrylamide and N-(5-bromoacetamidylpentyl) acrylamide (BRAPA), as described in WO 05/065814.

Certain embodiments of the disclosure may make use of solid supports that include an inert substrate or matrix (e.g. glass slides, polymer beads, etc.) which has been “functionalized,” for example by application of a layer or coating of an intermediate material including reactive groups which permit covalent attachment to biomolecules, such as polynucleotides. Examples of such supports include, but are not limited to, polyacrylamide hydrogels supported on an inert substrate such as glass. In such embodiments, the biomolecules (e.g. polynucleotides) may be directly covalently attached to the intermediate material (e.g. the hydrogel), but the intermediate material may itself be non-covalently attached to the substrate or matrix (e.g. the glass substrate). The term “covalent attachment to a solid support” is to be interpreted accordingly as encompassing this type of arrangement.

The pooled samples may be amplified on beads wherein each bead contains a forward and reverse amplification primer. In a particular embodiment, the library of indexed fragments is used to prepare clustered arrays of nucleic acid colonies, analogous to those described in U.S. Pub. No. 2005/0100900, U.S. Pat. No. 7,115,400, WO 00/18957 and WO 98/44151 by solid-phase amplification and more particularly solid phase isothermal amplification. The terms ‘cluster’ and ‘colony’ are used interchangeably herein to refer to a discrete site on a solid support including a plurality of identical immobilized nucleic acid strands and a plurality of identical immobilized complementary nucleic acid strands. The term “clustered array” refers to an array formed from such clusters or colonies. In this context, the term “array” is not to be understood as requiring an ordered arrangement of clusters.

The term “solid phase” or “surface” is used to mean either a planar array wherein primers are attached to a flat surface, for example, glass, silica or plastic microscope slides or similar flow cell devices; beads, wherein either one or two primers are attached to the beads and the beads are amplified; or an array of beads on a surface after the beads have been amplified.

Clustered arrays can be prepared using either a process of thermocycling, as described in WO 98/44151, or a process whereby the temperature is maintained as a constant, and the cycles of extension and denaturing are performed using changes of reagents. Such isothermal amplification methods are described in patent application numbers WO 02/46456 and U.S. Pub. No. 2008/0009420. Due to the lower temperatures useful in the isothermal process, this is particularly preferred in some embodiments.

It will be appreciated that any of the amplification methodologies described herein or generally known in the art may be used with universal or target-specific primers to amplify immobilized DNA fragments. Suitable methods for amplification include, but are not limited to, the polymerase chain reaction (PCR), strand displacement amplification (SDA), transcription mediated amplification (TMA) and nucleic acid sequence based amplification (NASBA), as described in U.S. Pat. No. 8,003,354. The above amplification methods may be employed to amplify one or more nucleic acids of interest. For example, PCR, including multiplex PCR, SDA, TMA, NASBA and the like may be used to amplify immobilized DNA fragments. In some embodiments, primers directed specifically to the polynucleotide of interest are included in the amplification reaction.

Other suitable methods for amplification of polynucleotides may include oligonucleotide extension and ligation, rolling circle amplification (RCA) (Lizardi et al., Nat. Genet. 19:225-232 (1998)) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835) technologies. It will be appreciated that these amplification methodologies may be designed to amplify immobilized DNA fragments. For example, in some embodiments, the amplification method may include ligation probe amplification or oligonucleotide ligation assay (OLA) reactions that contain primers directed specifically to the nucleic acid of interest. In some embodiments, the amplification method may include a primer extension-ligation reaction that contains primers directed specifically to the nucleic acid of interest. As a non-limiting example of primer extension and ligation primers that may be specifically designed to amplify a nucleic acid of interest, the amplification may include primers used for the GoldenGate assay (Illumina, Inc., San Diego, Calif.) as exemplified by U.S. Pat. Nos. 7,582,420 and 7,611,869.

DNA nanoballs can also be used in combination with methods and compositions as described herein. Methods for creating and utilizing DNA nanoballs for genomic sequencing can be found at, for example, US patents and publications U.S. Pat. No. 7,910,354, 2009/0264299, 2009/0011943, 2009/0005252, 2009/0155781, 2009/0118488 and as described in, for example, Drmanac et al., 2010, Science 327(5961): 78-81. Briefly, following genomic library DNA fragmentation adaptors are ligated to the fragments, the adapter ligated fragments are circularized by ligation with a circle ligase and rolling circle amplification is carried out (as described in Lizardi et al., 1998. Nat. Genet. 19:225-232 and US 2007/0099208 A1). The extended concatameric structure of the amplicons promotes coiling thereby creating compact DNA nanoballs. The DNA nanoballs can be captured on substrates, preferably to create an ordered or patterned array such that distance between each nanoball is maintained thereby allowing sequencing of the separate DNA nanoballs. In some embodiments, consecutive rounds of adapter ligation, amplification and digestion are carried out prior to circularization to produce head to tail constructs having several genomic DNA fragments separated by adapter sequences.

Exemplary isothermal amplification methods that may be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587. Other non-PCR-based methods that may be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyper-branched strand displacement amplification which is described in, for example Lage et al., Genome Res. 13:294-307 (2003). Isothermal amplification methods may be used with, for instance, the strand-displacing Phi 29 polymerase or Bst DNA polymerase large fragment, 5′->3′ exo- for random primer amplification of genomic DNA. The use of these polymerases takes advantage of their high processivity and strand displacing activity. High processivity allows the polymerases to produce fragments that are 10-20 kb in length. As set forth above, smaller fragments may be produced under isothermal conditions using polymerases having low processivity and strand-displacing activity such as Klenow polymerase. Additional description of amplification reactions, conditions and components are set forth in detail in the disclosure of U.S. Pat. No. 7,670,810.

Another polynucleotide amplification method that is useful in the present disclosure is Tagged PCR which uses a population of two-domain primers having a constant 5′ region followed by a random 3′ region as described, for example, in Grothues et al. Nucleic Acids Res. 21(5):1321-2 (1993). The first rounds of amplification are carried out to allow a multitude of initiations on heat denatured DNA based on individual hybridization from the randomly-synthesized 3′ region. Due to the nature of the 3′ region, the sites of initiation are contemplated to be random throughout the genome. Thereafter, the unbound primers may be removed and further replication may take place using primers complementary to the constant 5′ region.

In some embodiments, isothermal amplification can be performed using kinetic exclusion amplification (KEA), also referred to as exclusion amplification (ExAmp). A nucleic acid library of the present disclosure can be made using a method that includes a step of reacting an amplification reagent to produce a plurality of amplification sites that each includes a substantially clonal population of amplicons from an individual target nucleic acid that has seeded the site. In some embodiments, the amplification reaction proceeds until a sufficient number of amplicons are generated to fill the capacity of the respective amplification site. Filling an already seeded site to capacity in this way inhibits target nucleic acids from landing and amplifying at the site thereby producing a clonal population of amplicons at the site. In some embodiments, apparent clonality can be achieved even if an amplification site is not filled to capacity prior to a second target nucleic acid arriving at the site. Under some conditions, amplification of a first target nucleic acid can proceed to a point that a sufficient number of copies are made to effectively outcompete or overwhelm production of copies from a second target nucleic acid that is transported to the site. For example, in an embodiment that uses a bridge amplification process on a circular feature that is smaller than 500 nm in diameter, it has been determined that after 14 cycles of exponential amplification for a first target nucleic acid, contamination from a second target nucleic acid at the same site will produce an insufficient number of contaminating amplicons to adversely impact sequencing-by-synthesis analysis on an Illumina sequencing platform.

In some embodiments, amplification sites in an array can be, but need not be, entirely clonal. Rather, for some applications, an individual amplification site can be predominantly populated with amplicons from a first indexed fragment and can also have a low level of contaminating amplicons from a second target nucleic acid. An array can have one or more amplification sites that have a low level of contaminating amplicons so long as the level of contamination does not have an unacceptable impact on a subsequent use of the array. For example, when the array is to be used in a detection application, an acceptable level of contamination would be a level that does not impact signal to noise or resolution of the detection technique in an unacceptable way. Accordingly, apparent clonality will generally be relevant to a particular use or application of an array made by the methods set forth herein. Exemplary levels of contamination that can be acceptable at an individual amplification site for particular applications include, but are not limited to, at most 0.1%, 0.5%, 1%, 5%, 10% or 25% contaminating amplicons. An array can include one or more amplification sites having these exemplary levels of contaminating amplicons. For example, up to 5%, 10%, 25%, 50%, 75%, or even 100% of the amplification sites in an array can have some contaminating amplicons. It will be understood that in an array or other collection of sites, at least 50%, 75%, 80%, 85%, 90%, 95% or 99% or more of the sites can be clonal or apparently clonal.

In some embodiments, kinetic exclusion can occur when a process occurs at a sufficiently rapid rate to effectively exclude another event or process from occurring. Take for example the making of a nucleic acid array where sites of the array are randomly seeded with indexed fragments from a solution and copies of the indexed fragments are generated in an amplification process to fill each of the seeded sites to capacity. In accordance with the kinetic exclusion methods of the present disclosure, the seeding and amplification processes can proceed simultaneously under conditions where the amplification rate exceeds the seeding rate. As such, the relatively rapid rate at which copies are made at a site that has been seeded by a first target nucleic acid will effectively exclude a second nucleic acid from seeding the site for amplification. Kinetic exclusion amplification methods can be performed as described in detail in the disclosure of US Application Pub. No. 2013/0338042.

Kinetic exclusion can exploit a relatively slow rate for initiating amplification (e.g. a slow rate of making a first copy of an indexed fragment) vs. a relatively rapid rate for making subsequent copies of the indexed fragment (or of the first copy of the indexed fragment). In the example of the previous paragraph, kinetic exclusion occurs due to the relatively slow rate of indexed fragment seeding (e.g. relatively slow diffusion or transport) vs. the relatively rapid rate at which amplification occurs to fill the site with copies of the indexed fragment seed. In another exemplary embodiment, kinetic exclusion can occur due to a delay in the formation of a first copy of an indexed fragment that has seeded a site (e.g. delayed or slow activation) vs. the relatively rapid rate at which subsequent copies are made to fill the site. In this example, an individual site may have been seeded with several different indexed fragments (e.g. several indexed fragments can be present at each site prior to amplification). However, first copy formation for any given indexed fragment can be activated randomly such that the average rate of first copy formation is relatively slow compared to the rate at which subsequent copies are generated. In this case, although an individual site may have been seeded with several different indexed fragments, kinetic exclusion will allow only one of those indexed fragments to be amplified. More specifically, once a first indexed fragment has been activated for amplification, the site will rapidly fill to capacity with its copies, thereby preventing copies of a second indexed fragment from being made at the site.

In one embodiment, the method is carried out to simultaneously (i) transport indexed fragments to amplification sites at an average transport rate, and (ii) amplify the indexed fragments that are at the amplification sites at an average amplification rate, wherein the average amplification rate exceeds the average transport rate (U.S. Pat. No. 9,169,513). Accordingly, kinetic exclusion can be achieved in such embodiments by using a relatively slow rate of transport. For example, a sufficiently low concentration of indexed fragments can be selected to achieve a desired average transport rate, lower concentrations resulting in slower average rates of transport. Alternatively or additionally, a high viscosity solution and/or presence of molecular crowding reagents in the solution can be used to reduce transport rates. Examples of useful molecular crowding reagents include, but are not limited to, polyethylene glycol (PEG), ficoll, dextran, or polyvinyl alcohol. Exemplary molecular crowding reagents and formulations are set forth in U.S. Pat. No. 7,399,590, which is incorporated herein by reference. Another factor that can be adjusted to achieve a desired transport rate is the average size of the target nucleic acids.

An amplification reagent can include further components that facilitate amplicon formation and in some cases increase the rate of amplicon formation. An example is a recombinase. Recombinase can facilitate amplicon formation by allowing repeated invasion/extension. More specifically, recombinase can facilitate invasion of an indexed fragment by the polymerase and extension of a primer by the polymerase using the indexed fragment as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolyzable analogs thereof) in a recombinase-facilitated amplification reagent to facilitate amplification. A mixture of recombinase and single stranded binding (SSB) protein is particularly useful as SSB can further facilitate amplification. Exemplary formulations for recombinase-facilitated amplification include those sold commercially as TwistAmp kits by TwistDx (Cambridge, UK). Useful components of recombinase-facilitated amplification reagent and reaction conditions are set forth in U.S. Pat. Nos. 5,223,414 and 7,399,590.

Another example of a component that can be included in an amplification reagent to facilitate amplicon formation and in some cases to increase the rate of amplicon formation is a helicase. Helicase can facilitate amplicon formation by allowing a chain reaction of amplicon formation. The process can occur more rapidly than standard PCR since a denaturation cycle (e.g. via heating or chemical denaturation) is not required. As such, helicase-facilitated amplification can be carried out isothermally. A mixture of helicase and single stranded binding (SSB) protein is particularly useful as SSB can further facilitate amplification. Exemplary formulations for helicase-facilitated amplification include those sold commercially as IsoAmp kits from Biohelix (Beverly, Mass.). Further, examples of useful formulations that include a helicase protein are described in U.S. Pat. Nos. 7,399,590 and 7,829,284.

Yet another example of a component that can be included in an amplification reagent to facilitate amplicon formation and in some cases increase the rate of amplicon formation is an origin binding protein.

Methods of Sequencing

Following attachment of indexed fragments to a surface, the sequence of the immobilized and amplified indexed fragments is determined. The sequencing can be comprehensive or targeted. Comprehensive sequencing can be used when the entire sequence of each cell or nucleus present in the library is desired. Examples of applications that use comprehensive sequencing include, but are not limited to, whole genome sequencing, whole transcriptome sequencing, and ATAC sequencing. Targeted sequencing can be used when information regarding a biological feature is desired. In one embodiment, targeted sequencing can be used in the identification of a subpopulation of cells or nuclei, or subset of the genome, subset of the transcriptome, subset of the proteome, or any combination thereof, and is described in detail herein.

Sequencing can be carried out using any suitable sequencing technique, and methods for determining the sequence of immobilized and amplified indexed fragments, including strand re-synthesis, are known in the art and are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of an indexed fragment can be an automated process. Preferred embodiments include sequencing-by-synthesis (“SBS”) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

In one embodiment, a nucleotide monomer includes locked nucleic acids (LNAs) or bridged nucleic acids (BNAs). The use of LNAs or BNAs in a nucleotide monomer increases hybridization strength between a nucleotide monomer and a sequencing primer sequence present on an immobilized indexed fragment.

SBS can use nucleotide monomers that have a terminator moiety or those that lack any terminator moieties. Methods using nucleotide monomers lacking terminators include, for example, pyrosequencing and sequencing using γ-phosphate-labeled nucleotides, as set forth in further detail herein. In methods using nucleotide monomers lacking terminators, the number of nucleotides added in each cycle is generally variable and dependent upon the template sequence and the mode of nucleotide delivery. For SBS techniques that use nucleotide monomers having a terminator moiety, the terminator can be effectively irreversible under the sequencing conditions used as is the case for traditional Sanger sequencing which uses dideoxynucleotides, or the terminator can be reversible as is the case for sequencing methods developed by Solexa (now Illumina, Inc.).

SBS techniques can use nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments where two or more different nucleotides are present in a sequencing reagent, the different nucleotides can be distinguishable from each other, or alternatively the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026. This approach is being commercialized by Solexa (now Illumina Inc.), and is also described in WO 91/06678 and WO 07/123,744. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

In some reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth herein.

In particular embodiments some or all of the nucleotide monomers can include reversible terminators. In such embodiments, reversible terminators/cleavable fluorophores can include fluorophores linked to the ribose moiety via a 3′ ester linkage (Metzker, Genome Res. 15:1767-1776 (2005)). Other approaches have separated the terminator chemistry from the cleavage of the fluorescence label (Ruparel et al., Proc Natl Acad Sci USA 102: 5932-7 (2005)). Ruparel et al. described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluorophore and effectively reverses the termination. Examples of modified nucleotides are also described in U.S. Pat. Nos. 7,427,673, and 7,057,026.

Additional exemplary SBS systems and methods which can be used with the methods and systems described herein are described in U.S. Pub. Nos. 2007/0166705, 2006/0188901, 2006/0240439, 2006/0281109, 2012/0270305, and 2013/0260372, U.S. Pat. No. 7,057,026, PCT Publication No. WO 05/065814, U.S. Patent Application Publication No. 2005/0100900, and PCT Publication Nos. WO 06/064199 and WO 07/010,251.

Some embodiments can use detection of four different nucleotides using fewer than four different labels. For example, SBS can be performed using methods and systems described in the incorporated materials of U.S. Pub. No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can use sequencing by ligation techniques. Such techniques use DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be used with the methods and systems described herein are described in U.S. Pat. Nos. 6,969,488, 6,172,218, and 6,306,597.

Some embodiments can use nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003)). In such embodiments, the indexed fragment passes through a nanopore. The nanopore can be a synthetic pore or biological membrane protein, such as α-hemolysin. As the indexed fragment passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008)). Data obtained from nanopore sequencing can be stored, processed and analyzed as set forth herein. In particular, the data can be treated as an image in accordance with the exemplary treatment of optical images and other images that is set forth herein.

Some embodiments can use methods involving the real-time monitoring of DNA polymerase activity. Nucleotide incorporations can be detected through fluorescence resonance energy transfer (FRET) interactions between a fluorophore-bearing polymerase and 7-phosphate-labeled nucleotides as described, for example, in U.S. Pat. Nos. 7,329,492 and 7,211,414, or nucleotide incorporations can be detected with zero-mode waveguides as described, for example, in U.S. Pat. No. 7,315,019, and using fluorescent nucleotide analogs and engineered polymerases as described, for example, in U.S. Pat. No. 7,405,281 and U.S. Pub. No. 2008/0108082. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008)). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in U.S. Pub. Nos. 2009/0026082; 2009/0127589; 2010/0137143; and 2010/0282617. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different indexed fragments are manipulated simultaneously. In particular embodiments, different indexed fragments can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the indexed fragments can be in an array format. In an array format, the indexed fragments can be typically bound to a surface in a spatially distinguishable manner. The indexed fragments can be bound by direct covalent attachment, attachment to a bead or other particle or binding to a polymerase or other molecule that is attached to the surface. The array can include a single copy of an indexed fragment at each site (also referred to as a feature) or multiple copies having the same sequence can be present at each site or feature. Multiple copies can be produced by amplification methods such as, bridge amplification or emulsion PCR as described in further detail herein.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of cm², in parallel. Accordingly, the present disclosure provides integrated systems capable of preparing and detecting nucleic acids using techniques known in the art such as those exemplified herein. Thus, an integrated system of the present disclosure can include fluidic components capable of delivering amplification reagents and/or sequencing reagents to one or more immobilized indexed fragments, the system including components such as pumps, valves, reservoirs, fluidic lines and the like. A flow cell can be configured and/or used in an integrated system for detection of target nucleic acids. Exemplary flow cells are described, for example, in U.S. Pub. No. 2010/0111768 and U.S. Ser. No. 13/273,666. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.) and devices described in U.S. Ser. No. 13/273,666.

Detection of Rare Events

The present disclosure also provides methods for identifying and/or characterizing rare events. Currently, methods for characterization of rare events in a population without enrichment is costly and challenging. When enrichment is used, the selection is typically based on some biological feature of the cell such as size, morphology, or presence of an identifiable molecule like a protein or glycan on the cell's surface. This results in a limitation of the types of events that can be identified. The methods presented herein provide a significant advance in the ability to identify and/or characterize the presence of rare events. In general, the invention provides for identification, enrichment, and sequencing-based characterization of a subset of rare single cells present in a library of millions or billions of cells. The identification of rare single cells can be used to create a cellular database that can be used by researchers for determining which cells can be used in further analysis.

Examples of rare events include, but are not limited to, rare cells in a large population of cells. Types of rare cells include, but are not limited to, cell class, species type, and disease status or risk. Examples of rare cell classes include, but are not limited to, cells from an individual having an alteration in, for instance, the genome, transcriptome, or epigenome. Examples of rare species types include, but are not limited to, prokaryotic, eukaryotic, or fungal cells. Examples of rare cells associated with disease status or risk include, but are not limited to, cancer cells.

A rare event is typically identified by the presence of a biological feature, usually a nucleotide sequence, that correlates with the rare event. In one embodiment, a biological feature is a biomolecule, such as a protein, glycan, proteoglycan, or lipid. A biomolecule can be tagged with a nucleic acid that is attached to a compound, such as an antibody, that specifically binds the biomolecule. A biological feature can be known apriori (e.g., known before the method is practiced, also referred to as predetermined) or de novo (e.g., the biological feature is identified after a targeted or comprehensive sequencing described herein).

An example of a biological feature related to a genome includes, but is not limited to, an alteration in an immune cell, such as a gene rearrangement. An example of a biological feature related to a transcriptome includes expression of one or more specific genes or RNA molecules, or expression of a specific protein. Examples of biological features related to an epigenome include epigenetic patterns such as, but not limited to, methylation mark, methylation pattern, and accessible DNA, or expression of a specific protein that correlates with an epigenetic change. Examples of biological features that correlate with rare species types include 16s rRNA or rDNA, 18s rRNA or rDNA, and internal transcribed spacer (ITS) rRNA/rDNA, or expression of a specific protein by a rare species. Examples of biological features related to disease status or risk include germline or somatic cells having a variant DNA sequence or expression pattern of RNA and/or protein that correlates with a disease such as a cancer.

The method can include identifying members of a sequencing library—individual modified target nucleic acids—that contain a rare event. In one embodiment, the method can include interrogation of a sequencing library that is suspected of containing the rare event. Interrogating a sequencing library typically includes determining the sequence of two types of nucleotide regions present in the library; (i) the biological feature that correlates with the rare event, and (ii) the indexes present on the members of the library. In one embodiment, the sequence of more than one biological feature can be determined.

In one embodiment, the nucleotide sequence of the biological feature is identified by targeted sequencing. Methods for targeted sequencing are known in the art and can include the use of a primer that hybridizes near the biological feature in a location and orientation that serves as an initiation site for sequencing. For instance, when the biological feature is the presence of a specific single nucleotide polymorphism (SNP) a primer can be designed that will specifically anneal to nucleotides near the SNP. In another example, when the biological feature is a protein, a primer can be designed that will specifically anneal to nucleotides of the nucleic acid that is attached to a compound specifically bound to the biomolecule. The result is sequence data that allows the skilled worker to identify which members of the library include the biological feature of interest. Determining the sequence of the indexes present on members of a sequencing library is a routine part of single-cell combinatorial indexing methodologies.

The sequence data from the targeted sequencing of the biological feature and sequencing of the indexes is then analyzed using routine bioinformatic methods, and those combinations of index sequences that are present on the same library members as the biological feature are identified. This correlation of biological feature and index sequences results in the identification of a subset of members of the library, where each member includes the biological feature and a unique grouping of index sequences, and the creation of a cellular database. Each unique grouping of index sequences, also referred to herein as a “marker index sequence,” is likewise present on the other members of the library that are derived from the same cell or nucleus, e.g., indexed libraries of interest. In one embodiment, marker index sequences are contiguous indexes, i.e., sets of multiple indexes present on the library members in a row with 0, 1, 2, 3, 4 or more nucleotides between each of the indexes. As described herein, these marker index sequences can be used to focus subsequent sequencing efforts on those members of the library that are derived from the cells or nuclei that have the biological feature, and thereby reduce costs.

The method can further include altering the sequencing library to increase the representation of those members of the library that are derived from the cells or nuclei that have the biological feature. The altering can include enrichment (e.g., positive selection of those rare members of the library that include a desired marker index sequence) or depletion (e.g., negative selection, such as selective removal, of those abundant members of the library that do not include a desired marker index sequence).

Enrichment and depletion can include using the marker index sequences. Methods for enrichment and depletion are known in the art and include, but are not limited to, hybridization-based methods such as marker index sequence-specific amplification (e.g., adapter-anchored PCR), hybrid capture, and CRISPR (d) Cas9. Enrichment and depletion methods benefit from the use of nucleotide sequence that specifically hybridizes to desired marker index sequences. Thus, enrichment or depletion can be carried out on libraries containing contiguous indexes, i.e., the set of multiple indexes present on the library members in a row with 0, 1, 2, 3, 4 or more nucleotides between each of the indexes (see FIG. 5B). The contiguous indexes that correlate with the desired biological feature can be positively selected for and retained, resulting in enrichment of the desired library members. Alternatively, the contiguous indexes that do not correlate with the desired biological feature can be selected for and removed, resulting in depletion of library members that correlate to abundant cells and defacto enrichment of the library members that correlate with the desired biological feature. In one embodiment, enrichment can be coupled with targeted amplification. For instance, after construction of a sequencing library an amplification reaction can be used to specifically amplify the library members that contain the biological feature of interest. In one embodiment, specific amplification can be accomplished using a biological feature-specific primer designed to anneal to a nucleotide sequence having the biological feature and a second primer that anneals to one side of all members of the library. The biological feature-specific primer can include at its 5′ end one or more indexes and/or universal sequences.

The total length of a contiguous index is dependent on the size of the probe needed for specific hybridization between the probe and the members of the library having the desired marker index sequences. In some embodiments, the total length of a contiguous index (and therefore a marker index sequence) is at least 40, at least 45, at least 50, or at least 55 nucleotides, and no greater than 80, no greater than 75, no greater than 70, or no greater than 65 nucleotides. In one embodiment, the total length of a contiguous index is 60 nucleotides.

The use of either enrichment or depletion results in a sub-library that includes increased representation of those members of the library that are derived from the cells or nuclei that have the biological feature. Comprehensive sequencing of the sub-library can be carried out using routine methods, including those described herein. The increase in representation is high enough that comprehensive sequencing requires significantly less resources and is therefore cost-effective. The use of comprehensive sequencing of a sub-library can result in the identification of one or more additional previously unknown biological features.

Applications

The methods provided by the present disclosure can be easily integrated into essentially any application that includes sequencing library preparation, such as whole genome, transcriptome, epigenome, accessible (e.g., ATAC), and conformational state (e.g., HiC). A multitude of sequencing library methods are known to a skilled person that can be used in the construction of whole-genome or targeted libraries (see, for instance, Sequencing Methods Review, available on the world wide web at genomics.umn.edu/downloads/sequencing-methods-review.pdf).

In those embodiments directed to detecting rare events, the methods provided by the present disclosure can be easily integrated into essentially any application with single-cell combinatorial indexing (sci) methods including, but not limited to, whole genome (e.g., sci-WGS-seq), epigenome (e.g., sci-MET-seq), accessible (e.g., sci-ATAC-seq), transcriptome (sci-RNA-seq), and conformational (sci-HiC-seq). In some embodiments, an application includes use of a conformational single-cell combinatorial indexing that includes proximity ligation with linked-long read methodologies with cross-linking. In some embodiments, the application is a co-assay, where two or more different analytes or information from a sample are evaluated simultaneously. Examples of analytes include, but are not limited to, DNA, RNA, and protein (e.g., a surface protein). Examples include, but are not limited to, assays that analyze whole genome and transcriptome, or ATAC and transcriptome (Ma et al., 2020, bioRxiv, DOI: doi.org/10.1016/j.cell.2020.09.056).

In some embodiments, the application is metagenomics—the study of genetic material recovered directly from environmental samples. Examples of environments include those present in fields related to agriculture (e.g., soils), biofuels (e.g., microbial communities that convert biomass), biotechnology (e.g., microbial communities that produce biologically active compounds), and gut microbiota (e.g., microbial communities present in a human or animal microbiome). The genetic material can be present in prokaryotic and/or eukaryotic microbes (both uni- and multi-cellular), including fungal cells. The methods described herein can be used to identify rare cells whether or not they can be cultivated. Biological features that can be used to identify rare events in metagenomics include, but are not limited to, 16s rRNA or rDNA, 18s rRNA or rDNA, and internal transcribed spacer (ITS) rRNA/rDNA, or a protein encoded by a microbe. After identification, rare cells can be comprehensively sequenced.

In some embodiments, the application relates to disease status or risk. Rare events such as, but not limited to, single nucleotide polymorphisms (SNP) and/or biomarkers that correlate with disease or risk of disease, can be identified and those cells having the SNP and/or biomarker comprehensively sequenced. For example, a liquid biopsy of circulating cells in a subject's bloodstream or a tissue biopsy of cells can be analyzed for rare events related to disease or risk of disease. Rare events that can be assayed include, but are not limited to, somatic driver mutations, which can permit assignment of a specific cancer. A related application is fully characterizing and tracking tumor evolution by obtaining samples from a subject over an interval of time, selecting those cells or nuclei that are cancerous, and then comprehensively sequencing the subset of tumor cells.

In some embodiments, the application relates to immune cells. Immune cells undergo specific gene rearrangements related to the acquired immune system's ability to identify foreign molecules. Examples of immune cells that undergo gene rearrangements include, but are not limited to, T cells (e.g., rearrangement of T cell receptor), antigen presenting cells (e.g., rearrangement of genes encoding proteins of the major histocompatibility complex), and B cells (e.g., rearrangement of genes encoding antibody). A biological feature related to an alteration in an immune cell can be, but is not limited to, a specific rearrangement, or the protein resulting from a specific rearrangement. Immune cells having specific alterations can be fully characterized and tracked, including but not limited to T-cell receptor repertoire characterization and evolution. In another embodiment, the application relates to cell differentiation. For example, expression levels and/or methylation at different regions can be used to evaluate differentiation events such as correlations between accessibility and expression.

A non-limiting illustrative embodiment of the present disclosure is shown in FIG. 6. In this embodiment, a method for identification and characterization of T cell receptor repetoires can include providing a plurality of cells (FIG. 6, block 600), and distributing subsets of the cells into a plurality of compartments (FIG. 6, block 601). The plurality of cells can be from, for instance, a blood sample or a sample of lymph node. The nucleic acids present in the cells of each compartment are modified by insertion of an index (FIG. 6, block 602), and the cells are then pooled (FIG. 6, block 603). Additional indexes are added by “split and pool” steps of repeating the distributing (FIG. 6, block 601), index addition (FIG. 6, block 602), and pooling (FIG. 6, block 603) of the subsets. In one embodiment, each index is added to the same side of the members of the library to result in a contiguous index (see FIG. 5B). Optionally, a universal sequence can be added with one or more of the indexes. After addition of the last index the libraries of nucleic acids in the nuclei or cells can be pooled (FIG. 6, block 603) and further processed to prepare for targeted sequencing of the biological feature of interest, e.g., a biological feature that permits identification of T cell receptors that include a specific nucleotide sequence, such as one that can bind a biomolecule of a microbe or virus, and sequencing of the indexes associated with the biological feature of interest (FIG. 6, block 604). Sequence analysis (FIG. 6, block 605) is used to identify marker index sequences, i.e., the unique groupings of index sequences. The identified marker index sequences are (i) those that correlate with the biological feature and therefore identify the members of the library originating from the rare cells, or (ii) those that do not correlate with the biological feature and therefore identify the members of the library originating from the abundant cells. The following steps of this illustrative embodiment describe depletion of the abundant members of the library, but the method can be altered as described herein to include enrichment of the rare library members. Specific oligonucleotides or guide RNA sequences can be designed to hybridize with the marker index sequences that correlate with members of the library originating from the abundant cells (FIG. 6, block 606), and then used to deplete the sequencing library of the members originating from the abundant cells (FIG. 6, 607) by use of, for instance, hybridization capture or CRISPR digest. The result is an altered library containing increased representation of those members that originate from cells having the biological feature. The members of the altered sequencing library can be subjected to comprehensive sequencing (FIG. 6, block 608). Alternatively, the altered library can be subjected to additional rounds of enrichment and/or depletion until the representation of the desired members of the library is sufficient to meet characterization criteria. For instance, the members of the altered library can be sequenced a second time, marker index sequences identified, and specific oligonucleotides or guide RNA sequences designed and used to deplete or enrich the altered library.

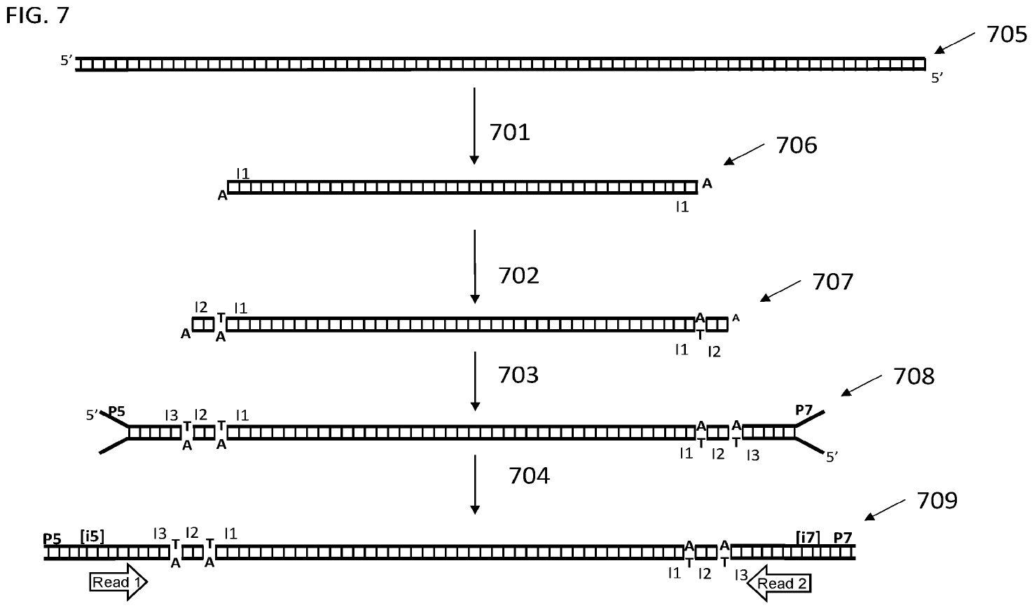

In some embodiments, the application includes the use of contiguous indexes. A non-limiting illustrative embodiment of an approach to producing a sequencing library with contiguous indexes is shown in FIG. 7. After distribution of subsets of cells or nuclei, a first compartment-specific index I1 can be added to the DNA molecules 705 present in the cells or nuclei, by, for instance, tagmentation (FIG. 7, step 701). When the primary source of nucleic acids is RNA, the nucleic acids can be converted to DNA using methods such as cDNA synthesis prior to tagmentation. The result is a library of modified nucleic acids present in the cells or nuclei, where each modified nucleic acid 706 includes a compartment-specific index I1 at each end. The subsets can be pooled and the ends of the resulting modified target nucleic acids can be repaired if necessary, for instance by 3′ fill-in. In one embodiment, the 5′ ends of the modified target nucleic acids can be phosphorylated. In one embodiment, the next step of second index addition can be facilitated by adding an overhang, e.g., a G, a C, or a poly-A tail, to the 3′ ends of the modified target nucleic acids. The pooled cells or nuclei can be distributed into a second set of compartments and a second compartment-specific index I2 added by, for instance, ligation of an adapter having an appropriately modified 3′ end, e.g., a T-tailed 3′ end (FIG. 7, step 702). This results in cells or nuclei containing a library of modified nucleic acids, where each modified nucleic acid 707 includes two compartment-specific indexes I1 and I2 at each end. The ends of the modified target nucleic acids can be altered to facilitate addition of the next index by, for instance, 5′ phosphorylation and/or modification of the 3′ ends by poly-A tailing or 3′ addition of G or C. The pooling and addition of another compartment-specific index can be repeated as desired to add the appropriate number of indexes. In one embodiment, an adapter with universal sequences can be included when the last compartment-specific index I3 is added to distributed subsets of cells or nuclei (FIG. 7, step 703). For instance, a mismatched adapter can be added to each end to result in modified nucleic acids 708. Examples of universal sequences include those used to immobilize the library members to an array (P5 and P7). The mismatched adapter can also include universal sequences useful for sequencing, or, in some embodiments, the modified nucleic acids 708 can be amplified (FIG. 7, step 704) and universal sequences useful for sequencing (i5 and i7) added to result in modified nucleic acids 709. The modified nucleic acids 709 can be used in targeted sequencing to identify marker index sequences that correlate with the biological feature useful for subsequent enrichment and/or deletion.

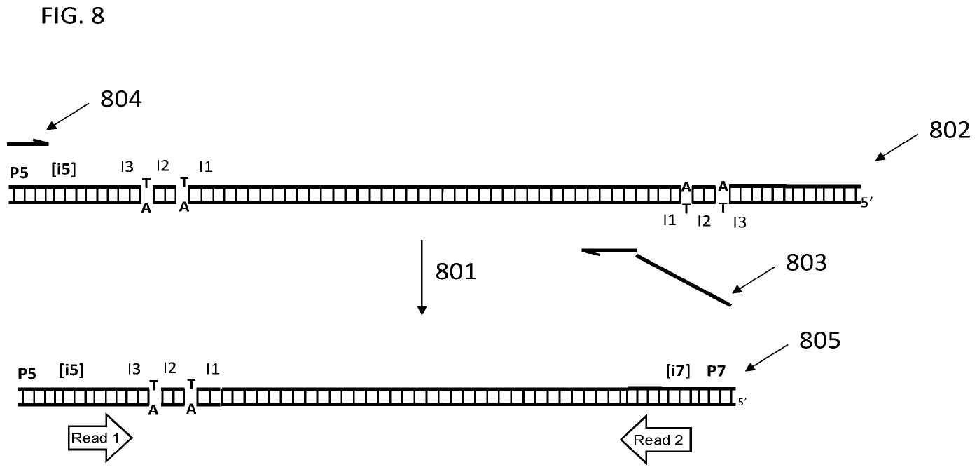

A non-limiting illustrative embodiment of coupling enrichment with targeted amplification is shown in FIG. 8. In this embodiment, a single-cell combinatorial library has been produced (e.g., FIG. 3, block 35; FIG. 4, block 47; FIG. 6, block 605) and the resulting modified nucleic acids (e.g., FIG. 7, modified nucleic acid 709) are subjected to an amplification reaction that specifically amplifies the library members that contain the biological feature of interest. The modified nucleic acids 802 having contiguous indexes are contacted with a primer 803 that can include two domains; a 3′ domain designed to anneal to a nucleotide sequence having the biological feature, and a 5′ domain having one or more universal sequences or the complement thereof, e.g., i7 and P7. The amplification reaction includes a second primer 804 that anneals to one side of all members of the library. Amplification 801 results in modified nucleic acids 805 having the compartment-specific indexes I1-3 at one end and, at the other end, the universal sequences added with the two-domain primer that targeted the biological feature. The amplified modified target nucleic acids can be used in targeted sequencing and sequencing to identify marker index sequences correlating with the biological feature of interest.

Also provided herein are kits. In one embodiment, a kit is for preparing a sequencing library. In one embodiment, the kit includes a transposome complex where the transposon recognition site such that a universal sequence can be inserted into a target nucleic acid. In another embodiment, the kit includes two transposome complexes where each complex includes a transposon recognition site with a different universal sequence, such that two universal sequences can be inserted into a target nucleic acid. In another embodiment, the kit includes the components to add at least one, two, or three indexes to nucleic acids. A kit can also include other components useful in producing a sequencing library. For instance, the kit can include at least one enzyme that mediates ligation, primer extension, or amplification for processing DNA molecules to include an index. The kit can include nucleic acids with index sequences.

The components of a kit are typically in a suitable packaging material in an amount sufficient for at least one assay or use. Optionally, other components can be included, such as buffers and solutions. Instructions for use of the packaged components are also typically included. As used herein, the phrase “packaging material” refers to one or more physical structures used to house the contents of the kit. The packaging material is constructed by routine methods, generally to provide a sterile, contaminant-free environment. The packaging material may have a label which indicates that the components can be used producing a sequencing library. In addition, the packaging material contains instructions indicating how the materials within the kit are employed. As used herein, the term “package” refers to a container such as glass, plastic, paper, foil, and the like, capable of holding within fixed limits the components of the kit. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

Compositions

During or following the production of sequencing libraries a number of molecules and compositions may result. For example, a molecule or composition that may result includes a modified target nucleic acid flanked on one or both sides by contiguous index. A contiguous index can include 1, 2, 3, 4, 5, 6, or more indexes in a row, where each index is separated from the other by 1, 2, 3, 4, or more nucleotides. In some embodiments, the total length of a contiguous index is at least 40, at least 45, at least 50, or at least 55 nucleotides, and no greater than 80, no greater than 75, no greater than 70, or no greater than 65 nucleotides. A library or a composition that includes a plurality of such modified target nucleic acids may result. Pooled libraries and compositions that include pooled libraries of such polynucleotides may result.

### EXEMPLARY EMBODIMENTS

Embodiment 1. A method for identifying a subpopulation of cells comprising a biological feature, the method comprising:

(a) providing a single-cell sequencing library,
wherein the sequencing library comprises a plurality of modified target nucleic acids,
wherein the modified target nucleic acids comprise at least one index sequence;
   (b) interrogating the sequencing library by targeted sequencing to identify the index sequences that are present on the same modified target nucleic acid as a biological feature,
wherein the index sequences associated with the biological feature are marker index sequences;
   (c) altering the sequencing library to obtain a sub-library,
wherein the sub-library comprises increased representation of the modified target nucleic acids comprising the marker index sequences in comparison to other modified target nucleic acids present in the sequencing library that do not comprise a marker index sequence;
   (d) determining the nucleotide sequence of the modified target nucleic acids comprising a marker index sequence.

Embodiment 2. The method of Embodiment 1, wherein the single-cell sequencing library comprises nucleic acids from multiple samples.

Embodiment 3. The method of any one of Embodiments 1-2, wherein the multiple samples comprise (i) samples of the same tissue obtained from different organisms, (ii) samples of different tissues from one organism, or (iii) samples of different tissues from different organisms.

Embodiment 4. The method of any one of Embodiments 1-3, wherein more than one marker index sequence is identified in step (b).

Embodiment 5. The method of any one of Embodiments 1-4, wherein the single-cell combinatorial sequencing library comprises target nucleic acids representative of the whole genome of the cells or nuclei or a subset of the genome.

Embodiment 6. The method of any one of Embodiments 1-5, wherein the subset of the genome comprises target nucleic acids representative of transcriptome, accessible chromatin, DNA, conformational state, or proteins of the cells or nuclei.

Embodiment 7. The method of any one of Embodiments 1-6, wherein the altering comprises enrichment of the modified target nucleic acids comprising the marker index sequences.

Embodiment 8. The method of any one of Embodiments 1-7, wherein the enriching comprises a hybridization-based method.

Embodiment 9. The method of any one of Embodiments 1-8, wherein the hybridization-based method comprises hybrid capture, amplification, or CRISPR (d) Cas9.

Embodiment 10. The method of any one of Embodiments 1-9, wherein the altering comprises depletion of the modified target nucleic acids that do not comprise the marker index sequences.

Embodiment 11. The method of any one of Embodiments 1-10, wherein the depletion comprises a hybridization-based method.

Embodiment 12. The method of any one of Embodiments 1-11, wherein the hybridization-based method comprises hybrid capture, amplification, or CRISPR (d) Cas9.

Embodiment 13. The method of any one of Embodiments 1-12, wherein the biological feature comprises a nucleotide sequence indicative of species type.

Embodiment 14. The method of any one of Embodiments 1-13, wherein the species type comprises the species of the cell.

Embodiment 15. The method of any one of Embodiments 1-14, wherein the biological feature comprises nucleotides of a 16s subunit, a 18s subunit, or an ITS non-transcriptional region.

Embodiment 16. The method of any one of Embodiments 1-15, wherein the biological feature comprises a nucleotide sequence indicative of cell class.

Embodiment 17. The method of any one of Embodiments 1-16, wherein the cell class comprises expression pattern, epigenetic pattern, immune gene recombination, or a combination thereof.

Embodiment 18. The method of any one of Embodiments 1-17, wherein the epigenetic pattern comprises methylation mark, methylation pattern, accessible DNA, or a combination thereof.

Embodiment 19. The method of any one of Embodiments 1-18, wherein the biological feature comprises a nucleotide sequence indicative of disease status or risk.

Embodiment 20. The method of any one of Embodiments 1-19, wherein disease status or risk comprises a variant DNA sequence, a variant expression pattern, or a variant epigenetic pattern that correlates with a disease.

Embodiment 21. The method of any one of Embodiments 1-20, wherein the variant DNA sequence comprises at least one single nucleotide polymorphism.

Embodiment 22. The method of any one of Embodiments 1-21, wherein the variant expression pattern comprises expression of a biomarker.

Embodiment 23. The method of any one of Embodiments 1-22, wherein the variant epigenetic pattern comprises a methylation mark, methylation pattern.

Embodiment 24. The method of any one of Embodiments 1-23, wherein the modified target nucleic acids comprise a contiguous index of at least 2 compartment-specific index sequences, wherein there are no greater than 6 nucleotides between the 2 index sequences.

Embodiment 25. The method of any one of Embodiments 1-24, wherein the contiguous index is present at each end of the modified target nucleic acids.

Embodiment 26. The method of any one of Embodiments 1-25, wherein the length of the contiguous index is at least 55 nucleotides.

Embodiment 27. The method of any one of Embodiments 1-26, wherein one copy of the contiguous index is present on the modified target nucleic acids.

Embodiment 28. The method of any one of Embodiments 1-27, wherein two copies of the contiguous index are present on the modified target nucleic acids.

Embodiment 29. The method of any one of Embodiments 1-28, wherein the plurality of modified target nucleic acids of the sequencing library is representative of at least 100,000 different cells or nuclei.

Embodiment 30. The method of any one of Embodiments 1-29, wherein the providing the single-cell combinatorial sequencing library comprises: processing a sample to produce a library, wherein the sample is a metagenomics sample obtained from an organism.

Embodiment 31. The method of any one of Embodiments 1-30, wherein the organism is a mammal.

Embodiment 32. The method of any one of Embodiments 1-31, wherein the metagenomics sample comprises a tissue suspected of comprising a commensal or pathogenic microbe.

Embodiment 33. The method of any one of Embodiments 1-32, wherein the microbe is prokaryotic or eukaryotic.

Embodiment 34. The method of any one of Embodiments 1-33, wherein the metagenomics sample comprises a microbiome sample.

Embodiment 35. The method of any one of Embodiments 1-34, wherein the providing the single-cell combinatorial sequencing library comprises:
processing a sample to produce a library, wherein the sample is from an organism.

Embodiment 36. The method of any one of Embodiments 1-35, wherein the organism is a mammal.

Embodiment 37. The method of any one of Embodiments 1-36, wherein the primary source of nucleic acids from the sample comprise RNA.

Embodiment 38 The method of any one of Embodiments 1-37, wherein the RNA comprises mRNA.

Embodiment 39. The method of any one of Embodiments 1-38, wherein the primary source of nucleic acids from the sample comprise DNA.

Embodiment 40. The method of any one of Embodiments 1-39, wherein the DNA comprises whole cell genomic DNA.

Embodiment 41. The method of any one of Embodiments 1-40, wherein the whole cell genomic DNA comprises nucleosomes.

Embodiment 42. The method of any one of Embodiments 1-41, wherein the primary source of nucleic acids from the sample comprise cell free DNA.

Embodiment 43. The method of any one of Embodiments 1-42, wherein the sample comprises cancer cells.

Embodiment 44. The method of any one of Embodiments 1-43, wherein the providing the single-cell combinatorial sequencing library comprises a producing the library with a single-cell combinatorial indexing method selected from single-nuclei transcriptome sequencing, single-cell transcriptome sequencing, single-cell transcriptome and transposon-accessible chromatin sequencing, whole genome sequencing of single nuclei, single nuclei sequencing of transposon accessible chromatin, single-cell epitope sequencing, sci-HiC, and sci-MET.

Embodiment 45. The method of any one of Embodiments 1-44, wherein the providing comprises providing two different single-cell combinatorial sequencing libraries from each cell or nucleus.

Embodiment 46. The method of any one of Embodiments 1-45, wherein the two different single-cell combinatorial sequencing libraries are selected from a single-cell combinatorial indexing method selected from single-nuclei transcriptome sequencing, single-cell transcriptome sequencing, single-cell transcriptome and transposon-accessible chromatin sequencing, whole genome sequencing of single nuclei, single nuclei sequencing of transposon accessible chromatin, sci-HiC, and sci-MET.

Embodiment 47. The method of any one of Embodiments 1-46, further comprising performing a sequencing procedure to determine the nucleotide sequences for the nucleic acids.

Embodiment 48. A method for preparing a sequencing library comprising nucleic acids from a plurality of single nuclei or cells, the method comprising:

(a) providing a plurality of nuclei or cells, wherein the nuclei or cells comprise nucleosomes;
(b) contacting the plurality of nuclei or cells with a transposome complex comprising a transposase and a universal sequence, wherein the contacting further comprises conditions suitable for incorporation of the universal sequence into DNA nucleic acids resulting in double stranded DNA nucleic acids comprising the universal sequence;
(d) distributing the plurality of nuclei or cells into a first plurality of compartments,
wherein each compartment comprises a subset of nuclei or cells;
   (e) processing DNA molecules in each subset of nuclei or cells to generate indexed nuclei or cells,
wherein the processing comprises adding to DNA nucleic acids present in each subset of nuclei or cells a first compartment specific index sequence to result in indexed nucleic acids present in indexed nuclei or cells,
wherein the processing comprises ligation, primer extension, hybridization, amplification, or a combination thereof, and
   (g) combining the indexed nuclei or cells to generate pooled indexed nuclei or cells.

Embodiment 49. The method of claim 48, wherein the providing comprises providing the plurality of nuclei or cells in a plurality of compartments, wherein each compartment comprises a subset of nuclei or cells, wherein the contacting comprises contacting each compartment with the transposome complex, and wherein the method further comprises combining the nuclei or cells after the contacting to generate pooled nuclei or cells.

Embodiment 50. The method of any one of Embodiments 48-49, wherein the providing comprises subjecting the nuclei to a chemical treatment to generate nucleosome-depleted nuclei while maintaining integrity of the isolated nuclei.

Embodiment 51. The method of any one of Embodiments 48-5048, further comprising:
distributing the pooled indexed nuclei or cells comprising the indexed nuclei or cells into a second plurality of compartments,
   wherein each compartment comprises a subset of nuclei or cells;
processing DNA molecules in each subset of nuclei or cells to generate dual-indexed nuclei or cells,
   wherein the processing comprises adding to DNA nucleic acids present in each subset of nuclei or cells a second compartment specific index sequence to result in dual-indexed nucleic acids present in indexed nuclei or cells,
   wherein the processing comprises ligation, primer extension, hybridization, amplification, or a combination thereof,
combining the dual-indexed nuclei or cells to generate pooled dual-indexed nuclei or cells;

Embodiment 52. The method of any one of Embodiments 48-51, further comprising:
distributing the pooled nuclei or cells comprising the dual-indexed nuclei or cells into a third plurality of compartments,
   wherein each compartment comprises a subset of nuclei or cells;
processing DNA molecules in each subset of nuclei or cells to generate triple-indexed nuclei or cells,
   wherein the processing comprises adding to DNA nucleic acids present in each subset of nuclei or cells a third compartment specific index sequence to result in triple-indexed nucleic acids present in indexed nuclei or cells,
   wherein the processing comprises ligation, primer extension, hybridization, amplification, or a combination thereof,
combining the triple-indexed nuclei or cells to generate pooled triple-indexed nuclei or cells.

Embodiment 53. The method of any one of Embodiments 48-52, wherein the distributing step comprises dilution.

Embodiment 54. The method of any one of Embodiments 48-53, wherein the compartment comprises a well, microfluidic compartment, or a droplet.

Embodiment 55. The method of any one of Embodiments 48-54, wherein compartments of the first plurality of compartments comprise from 50 to 100,000,000 nuclei or cells.

Embodiment 56. The method of any one of Embodiments 48-55, wherein compartments of the second plurality of compartments comprise from 50 to 100,000,000 nuclei or cells.

Embodiment 57. The method of any one of Embodiments 48-56, wherein compartments of the third plurality of compartments comprise from 50 to 100,000,000 nuclei or cells.

Embodiment 58. The method of any one of Embodiments 48-57, wherein the contacting comprises contacting each subset with two transposome complexes, wherein one transposome complex comprises a first transposase comprising a first universal sequence and a second transposome complex comprises a second transposase comprising a second universal sequence, wherein the contacting further comprises conditions suitable for incorporation of the first universal sequence and the second universal sequence into DNA nucleic acids resulting in double stranded DNA nucleic acids comprising the first and second universal sequences.

Embodiment 59. The method of any one of Embodiments 48-58, wherein the adding of the compartment specific index sequence comprises a two-step process of adding a nucleotide sequence comprising a universal sequence to the nucleic acids, and then adding the compartment specific index sequence to the nucleic acids.

Embodiment 60. The method of any one of Embodiments 48-59, further comprising obtaining the indexed nucleic acids from the pooled indexed nuclei or cells, thereby producing a sequencing library from the plurality of nuclei or cells.

Embodiment 61. The method of any one of Embodiments 48-60, further comprising obtaining the dual-indexed nucleic acids from the pooled dual-indexed nuclei or cells, thereby producing a sequencing library from the plurality of nuclei or cells.

Embodiment 62. The method of any one of Embodiments 48-61, further comprising obtaining the triple-indexed nucleic acids from the pooled triple-indexed nuclei or cells, thereby producing a sequencing library from the plurality of nuclei or cells.

Embodiment 63. The method of any one of Embodiments 48-62, further comprising:
providing a surface comprising a plurality of amplification sites,
   wherein the amplification sites comprise at least two populations of attached single stranded capture oligonucleotides having a free 3′ end, and
contacting the surface comprising amplification sites with the nucleic acid fragments comprising one, two, or three index sequences under conditions suitable to produce a plurality of amplification sites that each comprise a clonal population of amplicons from an individual fragment comprising a plurality of indexes.

Embodiment 64. A method for preparing a nucleic acid library comprising:
(a) providing a plurality of samples, wherein each sample comprises a plurality of cells or nuclei, wherein the plurality of cells or nuclei of each sample are present in one or more separate compartments;
(b) contacting the plurality of nuclei or cells with a transposome complex comprising a transposase and a universal sequence and with the proviso that the transposome complex does not comprise an index sequence, wherein the contacting further comprises conditions suitable for incorporation of the universal sequence into nucleic acids;
(c) adding a first index sequence to the nucleic acids of each separate compartment;
(d) combining the cells or nuclei of the separated compartments;
(e) distributing the cells or nuclei into a plurality of compartments; and
(f) adding a second index sequence to the nucleic acids of the plurality of compartments.

Embodiment 65. The method of Embodiment 64, wherein the first index sequence, the second index sequence, or the combination thereof, are added by ligation, primer extension, hybridization, amplification, or a combination thereof.

Embodiment 66. The method of any one of Embodiments 64-65, wherein steps (d)-(e) are repeated to add a third or more index sequences to the cells or nuclei of the plurality of compartments.

Embodiment 67. The method of any one of Embodiments 64-66, wherein the plurality of nuclei or cells are fixed.

Embodiment 68. The method of any one of Embodiments 64-67, further comprising an amplification of indexed nucleic acids after step (c) or step (f).

Embodiment 69. The method of any one of Embodiments 64-68, further comprising step (g) combining the nucleic acids of the plurality of compartments and determining the sequence of the nucleic acids.

Embodiment 70. The method of any one of Embodiments 64-69, further comprising performing a sequencing procedure to determine the nucleotide sequences for the nucleic acids.

Embodiment 71. A method for sequencing a single cell or nucleus comprising:
(a) uniquely indexing nucleic acids of each cell or nuclei in a sample, thereby generating an indexed library for each cell or nuclei;
(b) using a biological feature to identify one or more indexed libraries of interest from step (a);
(c) enriching the indexed libraries of interest of step (b) thereby generating an enriched library; and
(d) sequencing the enriched library from step (c).

Embodiment 72. The method of Embodiment 71, wherein the libraries are derived from DNA, RNA, or protein of the cells or nuclei.

Embodiment 73. The method of any one of Embodiments 64-72, wherein the biological feature is DNA, RNA, or protein or a combination thereof.

Embodiment 74. The method of any one of Embodiments 64-73, wherein the uniquely indexing in step (a) comprises associating at least two different indexes to the nucleic acids of the cells or nuclei.

Embodiment 75. The method of any one of Embodiments 64-74, wherein the at least two different indexes are a contiguous index.

Embodiment 76. The method of any one of Embodiments 64-75, wherein the enriched library is generated through positive enrichment.

Embodiment 77. The method of any one of Embodiments 64-76, wherein the positive enrichment comprises amplification.

Embodiment 78. The method of any one of Embodiments 64-77, wherein the positive enrichment comprises a capture agent.

Embodiment 79. The method of any one of Embodiments 64-78, wherein the positive enrichment comprises a solid support.

Embodiment 80. The method of any one of Embodiments 64-79, wherein the enriched library is generated through negative enrichment.

Embodiment 81. The method of any one of Embodiments 64-80, wherein the identifying the indexed library of interest in step (c) comprises sequencing the indexes.

Embodiment 82. A method for sequencing a single cell or nucleus comprising:
(a) providing a sample, wherein the sample comprises a plurality of nuclei or cells;
(b) associating a first index on each nucleus or cell in the sample;
(c) dividing the sample into a plurality of compartments;
(d) associating a second index on each nuclei or cell of the plurality of compartments;
(e) pooling the plurality of compartments;
(f) sequencing the pooled compartments;
(g) identifying a combination of first and second indexes associated with a biological feature;
(h) enriching the biological feature from the pooled compartments using the identified combination of first and second indexes from step (g).

Embodiment 83. A kit containing:
(a) a plurality of transposome complexes, wherein each transposome complex comprises a transposase and a transposon sequence, wherein the transposon sequence is not indexed;
(b) a first plurality of index oligonucleotides, wherein the first plurality of index oligonucleotides comprises oligonucleotides having at least two different sequences; and
(c) a ligase enzyme for use with the index oligonucleotides.

Embodiment 84. The kit of Embodiment 83, further comprising a second plurality of index oligonucleotides, wherein the second plurality of index oligonucleotides comprises oligonucleotide having different sequences from the first plurality of index oligonucleotides.

Embodiment 85. The kit of embodiment 83 or 84, further comprising a third plurality of index oligonucleotides, wherein the third plurality of index oligonucleotides comprises oligonucleotide having different sequences from the first plurality of index oligonucleotides and the second plurality of index oligonucleotides.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

A human cell atlas of chromatin accessibility during development

Abstract

The chromatin landscape of the human genome shapes cell type-specific programs of gene expression. We developed an improved assay for single cell profiling of chromatin accessibility based on three-level combinatorial indexing (sci-ATAC-seq3) and applied it to 59 fetal samples representing 15 organs, altogether profiling on the order of one million single cells. We leverage cell types defined by gene expression in the same organs to annotate these data, to build a catalog of hundreds-of-thousands of cell type-specific DNA regulatory elements, and to investigate the properties of lineage-specific transcription factors as well as cell type-specific enrichments of complex trait heritability. Together with the accompanying human cell atlas of gene expression during development, these data comprise a rich resource for the exploration of human biology.

Main Text

In recent years, single cell methods, experiments, and atlases have rapidly proliferated. However, the overwhelming majority of effort remains focused on single cell gene expression, which reflects only one aspect of cellular, developmental and organismal biology. Other aspects, including the chromatin landscape that shapes gene expression programs, are just as critical to investigate at single cell resolution, but are challenged by a relative paucity of scalable methods.

The framework of single cell combinatorial indexing (“sci”) involves the splitting and pooling of cells or nuclei to wells in which molecular barcodes are introduced in situ to the species of interest (e.g. RNA or chromatin) at each round. Through successive rounds of in situ molecular barcoding, species within the same cell are concordantly labeled with a unique combination of barcodes. sci—assays have been developed for profiling chromatin accessibility (sci-ATAC-seq), gene expression (sci-RNA-seq), nuclear architecture, genome sequence, methylation, histone marks and other phenomena, as well as sci—co-assays, e.g. for profiling chromatin accessibility and gene expression jointly (“CoBatch”, “Split-seq”, “Paired-seq”, and “dscATAC-seq” are methods that also rely on single cell combinatorial indexing).

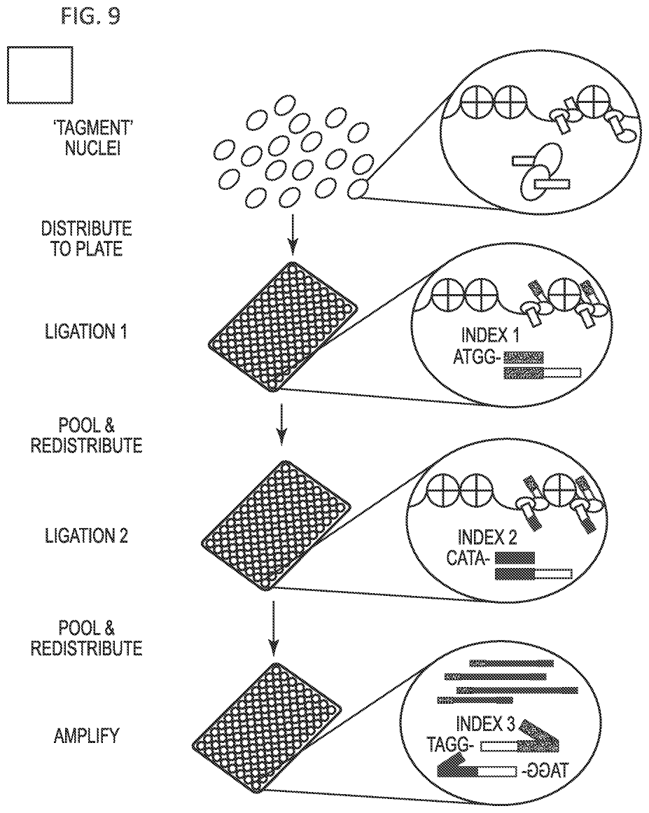

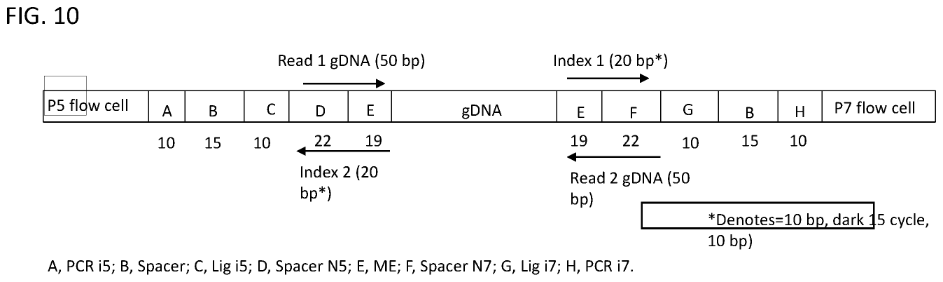

Although we were previously able to profile chromatin accessibility in ˜100,000 mammalian cells via two-level sci-ATAC-seq, the assay suffers from several limitations. For example, it requires custom-loading of the Tn5 enzyme with barcoded adapters, and is limited to 10⁴-10⁵ cells per experiment by collisions—cells receiving the same combination of barcodes. To address these issues, we developed an improved assay for single cell profiling of chromatin accessibility based on three levels of combinatorial indexing (sci-ATAC-seq3). In contrast with previous iterations of sci-ATAC-seq, the assay does not depend on molecularly barcoded Tn5 complexes (FIG. 9; FIG. 10). Rather, the first two rounds of indexing are achieved by ligation to either end of the conventional, uniformly loaded Tn5 transposase complex (standard “Nextera”), while the final round of indexing remains through PCR. Relative to two-level sci-ATAC-seq but similar to sci-RNA-seq3, sci-ATAC-seq3 substantially reduces the per-cell costs of library preparation as well as the rate of collisions. Theoretical collision rates for 2-level (96×384 wells) and 3-level indexing (384×384×384 wells) were 12% and 1.3% respectively, and the observed collision rate for a 3-level “species mixing” experiment using pooled equal numbers of GM12878 cells and CH12.LX cells was estimated as 4.0%, opening the door to experiments on the scale of 10⁶ cells. The protocol no longer requires cell sorting, and we also optimized ligase and polymerase choice, kinase concentration, and oligo designs and concentrations, to maximize the number of fragments recovered from each cell. Of note, while maintaining an enrichment in accessible regions, we made the explicit choice to maximize complexity at the expense of specificity for accessible sites. The estimated total unique reads (‘complexity’) for each cell was calculated using Picard, and the Fraction of Reads in Transcription Start Site (‘FRiTSS’) was calculated for each cell. Reads within 500 bp of a Gencode TSS were considered within the TSS. In particular, we found that the fixation conditions could be tuned to adjust the sensitivity (i.e., complexity) and specificity (i.e., enrichment in accessible sites) of the assay.

Towards a human cell atlas of chromatin accessibility, we applied sci-ATAC-seq3 to 59 fetal samples representing 15 organs (adrenal gland, two areas of cerebellum, eye, heart, intestine, kidney, liver, lung, muscle, pancreas, placenta, spleen, stomach, and thymus), altogether profiling chromatin accessibility in 1.6 million cells (FIG. 1D-E). In Example 2, we describe profiling of gene expression in 4 to 5 million cells from the same organs, based on an overlapping set of samples. The profiled organs span diverse systems; most notably absent are bone marrow, bone, gonads, and skin.

The rapid and uniform processing of heterogeneous fetal tissues represents a formidable challenge. We developed a new method for extracting nuclei directly from cryopreserved tissues that works well across a variety of tissue types and produces homogenates suitable for both sci-ATAC-seq3 and sci-RNA-seq3. In brief, we wrap flash-frozen tissue sections in aluminium foil and pulverize them into a powder using a chilled hammer and on dry ice. The tissue powder is then split into aliquots, one for sci-ATAC-seq3 and the other for sci-RNA-seq3.

For sci-ATAC-seq3, samples were obtained from 23 fetuses ranging from 89 to 125 days estimated gestational age. We lysed cells to isolate nuclei with published ATAC-seq cell lysis buffers and fixed the nuclei with formaldehyde before flash freezing for future processing. For nuclei from each tissue, approximately 50,000 fixed nuclei were deposited across 4 wells of a 96 well plate and processed for tagmentation. After tagmentation, the first index, which also identified the tissue sample, was introduced by ligation to one of the free ends of the asymmetric, inserted transposase complex. Following pooling and splitting, the second index was introduced by ligation to the other free end of the transposase complex. Following another round of pooling and splitting, the final index was appended by PCR and the resulting amplicons pooled for sequencing.

We sequenced sci-ATAC-seq3 libraries from 3 experiments across 5 Illumina NovaSeq runs, altogether generating over 50 billion reads. As an initial QC check, we examined our data at the tissue level, i.e. prior to splitting it to single cells. We downloaded and remapped all available single-ended DNase-seq samples from fetal tissues from the ENCODE data portal. We then identified peaks of accessibility in each of our “pseudobulk” samples and each ENCODE sample, merged these sets, and scored each sample for accessibility at each peak in the master list. Although the sci-ATAC-seq3 data was somewhat less enriched in peaks (median reads in peaks: 29% for sci-ATAC-seq3; 35% for ENCODE DNase-seq, samples from the same tissue were comparably correlated for the two assays (median Spearman correlation: 0.93 for two samples from the same tissue for sci-ATAC-seq3; 0.91 for DNase-seq) with greater technical reproducibility for sci-ATAC-seq3 (median Spearman correlation: 0.95). Furthermore, samples clustered into their respective tissues based on these aggregate profiles, whether analyzing the sci-ATAC-seq3 samples alone or the sci-ATAC-seq3 and DNase-seq samples together using pairwise Spearman correlations to cluster samples.

Upon splitting reads based on cellular barcodes and applying a dynamic threshold as previously described, we identified 1,568,018 cells. From the barnyard control, we estimate a collision rate of ˜5% for each of the three experiments. Uniform Manifold Approximation and Projection (UMAP) visualization of cells corresponding to the human sentinel tissue did not reveal any obvious experimental batch effects. Three samples were dropped on account of poor nucleosomal banding of their fragment size distribution; a further two samples were dropped because very few cells were captured. We estimate that we sequenced a median of 91% to 99% of all unique fragments per cell per tissue type in these sci-ATAC-seq3 libraries.

We identified peaks of accessibility on a tissue-by-tissue basis then merged these to generate a master set of 1.05 million sites. After scoring each cell for the presence or absence of reads at each site, we filtered out lower-quality cells on the basis of the number of total unique reads (sample-specific minimums ranging from 1,000 to 3,586), the fraction of reads overlapping the master set of accessible sites (sample-specific minimums ranging from 0.2 to 0.4), the fraction of reads falling near a TSS (+/−1 kb; sample-specific minimums ranging from 0.05 to 0.15), and a doublet score derived from an adaptation of the Scrublet doublet detection algorithm initially developed for scRNA-seq data (excluding the ˜10% of cells with the highest doublet scores).

After these procedures, 790,957 single cell chromatin accessibility profiles from 54 fetal samples remained. The total number of high-quality cells per tissue ranged from 2,421 for spleen to 211,450 for liver. The median number of unique fragments per cell for this set is 6,042, with a median of 0.49 overlapping the master set of accessible sites and 0.19 falling near a TSS (+/−1 kb).

We subjected high-quality cells to latent semantic indexing (LSI) on a tissue-by-tissue basis, using a log transformed term frequency component. Although we did not observe obvious evidence of batch effects for different samples corresponding to the same tissue, we applied the Harmony algorithm to align samples within PCA space for each tissue as a conservative measure. Using the aligned PCA space for each tissue, we then applied Louvain clustering, initially obtaining 172 clusters across all tissues. We further reduced the dimensionality of each tissue dataset using UMAP.

Annotating Cell Types

As we and others have shown, the annotation of cell types in scATAC-seq datasets can be greatly simplified by leveraging scRNA-seq datasets. In order to partially automate cell type annotations for our scATAC-seq data, we first annotated cell types within our scRNA-seq data for the same tissues, as described in the companion manuscript. Second, we computed gene-level accessibility scores for our scATAC-seq data, aggregating the number of transposition events falling within gene bodies extended by 2 kb upstream of their TSS. Third, we used the gene-by-cell matrices for each data type as input to an approach for finding likely correspondences between scRNA-seq and scATAC-seq clusters based on non-negative least squares (NNLS) regression, which resulted in an initial “lift-over” set of automated annotations for our scATAC-seq clusters. Finally, we manually reviewed all automated annotations by examining pileups around marker genes for each cell type within each tissue, making modifications to assigned labels as deemed necessary. Cell types were first annotated in sci-RNA-seq data gathered on matching tissues based on marker gene expression. Louvain clusters were identified in ATAC data for each tissue. Next, gene level accessibility scores were calculated for each of these clusters and matched to RNA clusters based on non-negative least squares (NNLS) regression, in some cases leading to merging of Louvain clusters. These first-pass automated annotations were further refined by manually reviewing the cluster-specific accessibility landscape around marker genes. Annotated cell types showed specific accessibility around the TSSs of known marker genes. For each cell type or unannotated cluster, accessibility near the TSS of known marker genes was summed and the scale was normalized to account for differences in total reads per cell as well as cell numbers across cell types. The data implied that some unannotated clusters might not represent novel cell types but rather technical artifacts (e.g., doublets). We note that while other approaches have shown great promise for multi-modal integration of single cell data, we found the cluster-to-cluster NNLS method sufficient for our purposes here and much less computationally intensive.

Altogether, we were able to annotate 150 of the 172 clusters (87%), or 163 of 172 (95%) if we include lower-confidence labels. Some clusters received the same annotation within the same tissue and were thus merged, resulting in 124 annotations across all tissues. Of these, some annotations were present across multiple tissues (e.g. erythroblasts in 4 tissues). Collapsing across tissues resulted in 54 unique cell type annotations that map 1:1 to annotations made in our scRNA-seq dataset (or 59 if we include lower-confidence labels and 1:2 mappings). Many of the scRNA-seq cell types that were not found in the chromatin accessibility data at this level of resolution are small clusters that may not have been sufficiently sampled to be detectable, due to the lower number of cells profiled in this study (˜4M (RNA) vs. ˜800K (ATAC) high-quality cells). On the other hand, most of the 9 scATAC-seq clusters that remained fully unannotated appear to be due to unfiltered doublets, as they are characterized by accessibility in marker genes for multiple adjacent cell types in the UMAP representation.

Identifying Lineage-Specific TFs

We next sought to integrate and compare chromatin accessibility in cell types across all 15 organs. To mitigate the effects of gross differences in the numbers of cells per organ and/or cell type, we randomly sampled 800 cells per cell type per organ (or in cases where fewer than 800 cells of a given cell type were represented in a given organ, all cells were taken), and performed UMAP visualization. Reassuringly, cell types represented in multiple organs clustered together, e.g. stromal cells (9 organs), endothelial cells (13 organs), lymphoid cells (7 organs) and myeloid cells (10 organs), rather than by batch or individual. Developmentally and functionally related cell types colocalized as well, e.g. diverse blood cells, secretory cells, PNS neurons, CNS neurons.

A key question in developmental biology is which transcription factors (TFs) are responsible for generating this diversity of cell types from an invariant genome. We next sought to leverage the breadth of this human cell atlas of chromatin accessibility to systematically assess which TF motifs are differentially accessible and thus nominate key regulators of cell fate in the context of in vivo human development.

As a first approach, we used a linear regression model to ask which TF motifs found in the accessible sites of each cell best explain its cell type affiliation. Initially treating each tissue independently, we identified the most highly enriched motifs/TFs from the JASPAR database in each of 124 annotated cell type clusters, which revealed both known and potentially novel regulators. For example, in the placenta, the motif of SPI1/PU.1, an established regulator of myeloid lineage development, is highly enriched in peaks of myeloid cells; The motif of TWIST-1, which is required for formation of stromal progenitors, is enriched in peaks of stromal cells; the FOS::JUN motif is associated with chromatin accessibility in extravillous trophoblasts, a cell type where the corresponding API complex has been described to be specifically active.

Interestingly, an unannotated cluster within the placenta was strongly enriched for GATA1::TAL1 motifs, established regulators of erythropoiesis. These cells clustered with erythroblasts from other tissues in the global UMAP and upon further inspection, key erythroid marker genes exhibited specific promoter accessibility. In the NNLS-guided workflow, this cluster was not annotated, because an erythroblast cluster was not detected in the placenta in the scRNA-seq study, possibly because the placenta is one of the few tissues where we have more ATAC than RNA cells. Thus, motif enrichment can assist in cell type annotation, if the key regulators of a cell type are known.

We repeated this analysis on the 54 main cell types observed across all tissues, i.e. after collapsing cell types appearing in multiple tissues. As expected, the top motifs remained consistent with the tissue-specific analyses as well as the literature, e.g. SPI1/PU.1 in myeloid cells; CRX in retinal pigment and photoreceptor cells; MEF2B in cardiomyocytes and skeletal muscle cells (31); and SRF in endocardial and smooth muscle cells. While most motifs are enriched in only one or two cell types, neuronal TF motifs including OLIG2, NEUROG1 and POU4F1 are enriched in multiple neuronal cell types. Another notable exception is HNF1B, conventionally associated with kidney and pancreas development, whose motif is enriched in 13 cell types spanning a range of specialized epithelial and secretory cells.

POU2F1 is an example of a TF that has not previously been associated with a particular developmental branch but rather has been suggested to be an exception within the POU family—broadly expressed and controlling no specific trajectory. In contrast, we find that at least in human fetal development, its motif is enriched in several neuronal cell types. Lending further support, POU2F1 is specifically expressed in those same cell types.

Extending on this observation, we next sought to leverage the companion scRNA-seq atlas to more generally ask whether TFs are differentially expressed in a pattern consistent with the differential accessibility of their motifs. For example, looking across all cell types annotated in same tissue in both datasets, the expression of the myeloid pioneer factor SPI1/PU.1 is strongly positively correlated with the enrichment of its motif at accessible sites. Intriguingly, this analysis also revealed many TFs with a negative correlation between their expression and motif enrichment. Upon closer inspection, these TFs tended to be repressors. For example, GFI1B has been described to act as a repressor crucial to erythroblast and megakaryocyte development by recruiting histone deacetylase upon binding its motif and inducing closing of the chromatin, e.g. at the embryonic hemoglobin locus. Consistent with this, we observe its expression to be negatively correlated with its motif enrichment at accessible sites.

Categorizing TFs as ‘activators’ or ‘repressors’ based upon GO terms, we find that TF expression and motif accessibility tend to be positively correlated for annotated activators, and negatively correlated for annotated repressors, and correlation of motif enrichment and expression can be used to predict the mode of action of unclassified TFs. Exceptions can largely be explained by missing or conflicting GO terms, whereas a literature search puts them into the category predicted by the correlation value. Accordingly, this kind of analysis may provide a systematic approach for classifying TFs as activators or repressors. For example, NFATc3 is generally described as an activator, but our analysis points towards a repressive mode of action, especially in developing T cells where it is highly expressed yet its motif is depleted in accessible sites. Such a repressive mode of action for NFATc3 has been hinted at in previous publications. Apart from a general classification, we can also gain insight into the cell type contexts in which a TF might variably act as an activator or repressor. For example, TFs including FOXO3 have been proposed to act as activators in their unmodified state but as repressors when phosphorylated, which might explain its more ambiguous relationship between expression and accessibility.

The above approach allows us to systematically associate known TFs with potentially novel roles, has the advantage that it does not rely on preselecting differentially accessible sites for each cell type, and the further advantage that we can relate the expression of a TF with the accessibility of its corresponding motif. However, it is limited in that we are relying on databases of known TF motifs. As a different approach, we also calculated specificity scores for each accessible site, selected the 2,000 most specific peaks for each cell type, and searched de novo for enriched motifs within this set compared to CpG-matched background genomic sequences. In general, the top de novo motifs for individual cell types agree with the top known motifs identified by linear regression. Interestingly, some cell types that did not have strong matches to known motifs (e.g. endothelial, stromal, Schwann cells) were nonetheless strongly associated with de novo motifs. For endothelial cells in particular, such results are discussed further below.

Cross-Tissue Analyses of Blood Cells and Endothelial Cells

The nature of this dataset creates an opportunity to investigate organ-specific differences in chromatin accessibility within broadly appearing cell types, e.g. blood cells and endothelial cells. In our first pass of cell type annotations for the blood system, we were able to differentiate between myeloid cells, lymphoid cells, erythroblasts, megakaryocytes and hematopoietic stem cells. Extracting and reclustering these blood lineages from all organs allowed us to additionally identify macrophages, B cells, NK/ILC 3 cells, T cells and dendritic cells, once again adopting an RNA-assisted annotation approach (of note, analyzing similar cell types from multiple tissues necessitated an additional doublet cleaning step; see Methods). Macrophages could be further separated into groups associated with tissue-of-origin, as has been previously observed, as well as phagocytic macrophages. This latter group was identified mainly in the spleen, followed by the liver and the adrenal gland. Of particular interest within the blood lineages are the erythroblasts, due to the spatiotemporal dynamics of erythropoiesis during fetal development. We initially detected this lineage in the liver, adrenal gland, heart and placenta; our cross-tissue analysis additionally identified erythroblasts in the shallowly profiled spleen (where only megakaryocytes and myeloid cells were annotated originally). The ratio of erythroblasts within the blood lineages of a tissue is highest in the liver, in line with this organ being the primary site of erythropoiesis at this developmental stage, followed by the spleen and adrenal gland, phenocopying the trend observed in the RNA data. The unexpected observation of the adrenal gland as a potential site of fetal hematopoiesis is discussed further in Example 2.

Further investigating erythroblasts, we observe that regions proximal to both the adult beta- and fetal gamma-globin genes are accessible at this stage of development, whereas the embryonic epsilon-globin gene's promoter is inaccessible. The erythroblast cluster could be further subdivided into five major Louvain clusters with differential chromatin accessibility, including a distinct erythroblast progenitor cluster. Accessible sites in the erythroblast progenitor cluster as well as the adjacent early erythroblast cluster (erythroblast_3), are enriched for GATA1::TAL1 as well as other GATA motifs. Comparison of expression levels of various GATA factors in erythroblast progenitors allows us to nominate GATA1/2 as the likely TFs responsible for this motif enrichment. The other erythroblast clusters, corresponding to later stages of erythropoiesis, show motif enrichment for NFE2/NFE2L2 (erythroblast_1) and KLF factors (erythroblast_2/4) and notably a marked absence of enrichment for GATA motif accessibility. A recently published scRNA-seq study on the murine hematopoietic system reported induction of GATA2 early in erythropoiesis, with subsequent decrease in GATA2 yet stable GATA1 expression. In contrast, a study of sorted bulk human in vitro cultured erythroid populations revealed a decrease in GATA1 expression from progenitors to differentiated erythroblasts, in line with what we observe in human fetal tissues, as well as increased KLF1 and NFE-2 levels in later stage erythroblasts. Our results further indicate that there might be epigenetically distinct subpopulations of differentiated erythroblasts in which the accessibility landscape is shaped by non-GATA factors such as KLF1 or NFE-2. For example, a distal regulatory element upstream of GYPA, which is used as erythrocyte invasion receptor by the malaria parasite, is most accessible in the erythroblast_1 population and contains a motif resembling the NFE-2 motif.

Another interesting cross-tissue system is the vascular endothelium. Interestingly, no TF has been described to be exclusively expressed in vascular endothelial cells, leading to the suggestion that the endothelial-specific transcriptome is controlled combinatorially by several TFs that have overlapping expression in the endothelium. Consistent with this, we fail to observe any single, strong enrichment in endothelial cells in our analysis of JASPAR motifs. On the other hand, de novo motif discovery on the 2,000 most endothelial-specific peaks revealed strong enrichment over background genomic sequences for motifs resembling ERG and SOX15. These motifs were likely not weighted as strongly in our linear modeling approach because they are not restricted to endothelial cells (the ERG motif being more enriched in megakaryocytes; and SOX15 being enriched in several cell types), nor is expression of these TFs limited to this cell type. In line with this, ERG has been previously described as a major regulator of endothelial function, but also drives transdifferentiation into megakaryocytes.

Endothelial cells exist in all organs, where they need to perform both constitutive and highly specialized functions, such as gas exchange in the lung or fluid filtration in the kidney. In our study we detect endothelial cells in 13 out of 15 organs (the exceptions being the more shallowly profiled cerebellum and eye). Extracting these cells across organs and reclustering revealed a marked separation according to tissue-of-origin, in spite of stringent iterative filtering steps to remove any residual contaminating doublets (Methods) and in contrast to the erythroblast lineage. Consistent with this, we also observe tissue-specific programs of gene expression, as described in Example 2. Indeed, peaks of accessibility closest to these differentially expressed genes have a higher specificity score in the matching tissue in the ATAC data. Furthermore, endothelial cells derived from nearly all organs exhibited specific TF motif enrichments. Of note, the TFs for many of the enriched motifs are also differentially expressed in the matching tissue in the RNA data.

Overall, these findings indicate that the general program of chromatin accessibility and gene expression in endothelial cells, a widely distributed cell type that needs to fill both general and organ-specific functions, is mediated by a combination of constitutive TFs like ERG and SOX15, as well as tissue-specific TFs that drive additional specialization. These analyses also highlight the merit of combining both de novo motif enrichment in specific peaks and linear model approaches across tissues to nominate the key regulators underlying the chromatin accessibility landscape of individual cell types.

Another intriguing example involves the PAEP_MECOM positive cell type in placenta, identified in both the scRNA-seq and sc-ATAC-seq atlases. Regulatory regions in this lineage are strongly enriched for the motif of HNF1B, a factor that is conventionally associated with kidney and pancreas development. For example, HNF1B is highly specifically expressed in the PAEP_MECOM cell lineage within the placenta. The nature of ATAC-seq data, which captures some genomic reads even in inaccessible sites across entire chromosomes, allows sexing of cells on the basis of Y chromosome over X chromosome or autosomally-derived reads. Interestingly, we find that the PAEP_MECOM and IGFBP1_DKK positive placental cell types, as well as to a lesser extent placental myeloid cells, have a significantly lower Y chromosome read ratio in male fetuses. Consistent with what is known about PAEP (glycodelin) and IGFBP1, these cell types potentially correspond to maternal endometrial epithelial and stromal cells, respectively.

CICERO

As a resource for further study, we generated Cicero coaccessibility scores and Cicero gene activity scores for each tissue in the dataset. Cicero coaccessibility scores can be used to predict cis-regulatory interactions between accessible elements. We combined the elements paired by positive coaccessibility scores to create a database of putative cis-regulatory interactions. This database includes 80 million unique co-accessible pairs including 4.5 million (6%) promoter-distal pairs, 76 million (94%) distal-distal pairs and 128,000 (0.2%) promoter-promoter pairs. We found an average of 33 million coaccessible pairs per tissue. 38% of pairs were unique to only a single tissue, while only 0.007% of pairs were detected in all 16 tissues. Pairs found in more tissues were more likely to be promoter-distal and promoter-promoter. The generated coaccessibility scores and gene activity scores are available for download on our website.

Of note, 89% of the 436,206 initially identified sites were significantly differentially accessible (DA) at a false discovery rate (FDR) of 1% in at least one of these 85 cell clusters relative to a control set of 2,040 cells (120 cells randomly sampled from each of the 17 samples; see Additional Resources). To identify DA sites at which accessibility was restricted to specific cluster(s), we adapted a metric for quantifying gene expression specificity in scRNA-seq studiesto chromatin accessibility and calculated it for all 436,206 sites by all 85 clusters. We classified 39% (167,981/436,206) of accessible sites as cluster restricted (i.e., increased accessibility in a limited number of clusters); 55% (92,334/167,981) of these were restricted to a single cluster.

Implicating Cell Types in Common Human Traits and Diseases

A major fraction of heritability for common human traits and diseases, as measured by genome-wide association studies (GWASs), partitions to distal regulatory elements, which are often cell-type specific. Consequently, much work has gone into intersecting GWAS signals with bulk DNase hypersensitivity data (and other epigenetic features), with the goal of systematically linking particular diseases to the dysfunction of specific tissues. However, the resolution of such studies is markedly limited by cell-type heterogeneity. Given the degree of conservation of chromatin accessibility between mice and humans, we wondered if we could use our data to better understand the cell-type-specific effects of genetic variation underlying complex human traits regardless of the differences between species. Therefore, despite the fact that our data were generated on mouse tissues, we sought to apply state-of-the-art methods for detecting cell-type-specific enrichment of human heritability.

To do so, we quantified the enrichment of heritability for human traits within DA peaks for each of our 85 clusters using partitioned linkage disequilibrium (LD) score regression (LDSC). After lifting over human SNPs to orthologous coordinates in the mouse genome, we calculated the enrichment of heritability for 32 phenotypes across the DA peaks obtained for each of our 85 clusters. 55 of the 85 cell types had an enrichment for at least one phenotype, while 28 of 32 phenotypes were enriched for at least one cell type. As a broad trend, we observed a strong enrichment of heritability for autoimmune diseases such as lupus, celiac disease, and Crohn's disease in clusters corresponding to leukocytes, whereas for neurological traits such as bipolar disorder, educational attainment, and schizophrenia, the enrichments occurred in neuronal cell types. Notably, most of these enrichments were not apparent in the peaks called from bulk tissues, demonstrating the value of cell types defined by single-cell chromatin accessibility data. Many enrichments were consistent with expectation. For example, the strongest enrichments of heritability for low-density lipoprotein (LDL) cholesterol, high-density lipoprotein (HDL) cholesterol, and triglycerides are in hepatocytes, although interestingly, LDL cholesterol was also significant in the kidney epithelium of the loop of Henle. Likewise, the strongest enrichment of heritability for immunoglobin A (IgA) deficiency are in clusters of T cells. These signals can also lead to refined understandings of the importance of subtypes of cells. As an example of this trend, although enrichments of heritability for bipolar disorder are observed for multiple neuronal clusters, the strongest enrichments involve excitatory neurons. In contrast, heritability for Alzheimer's disease is not enriched in any class of neurons. Instead, its strongest enrichment is found in a cluster of microglia.

To expand our analysis to a larger set of traits, we downloaded summary statistics (nealelab.github.io/UKBB_ldsc/) for GWASs for 2,419 traits in over 300,000 individuals from the UK Biobank. Focusing on 405 traits with an effective sample size of ≥5,000 and estimated heritability of ≥0.01, we observed significant enrichment of heritability in 273 traits in at least one cell type, while 74 of the 85 cell types exhibit enriched heritability for at least one trait. While the same broad trends as described above are also seen here for autoimmune and neurological traits, the much larger number of traits measured by the UK Biobank reveals additional trends. For example, many measures of body size and composition (e.g., body mass index) are also associated with cell types in the brain (FIG. 18 B). Additionally, specific subsets of T cells (12.1, 12.2) are more associated with asthma and allergic rhinitis than other cell types, including other T cell clusters. At a more granular level, heart attacks are associated with endothelial cells from the liver (25.3), but not from other endothelial clusters, while gout is associated with kidney proximal tubule cells. The framework that we demonstrate here can be readily applied to single-cell chromatin accessibility data collected from any human or mouse tissue and any heritable trait.

One consequence of the new design is that it is compatible with both 2-level (‘2lv2’ or ‘2-level version 2 protocol’) and 3-level (‘3lv2’) configurations, providing more flexibility for study design (FIG. 9).

Finally, we also tested various conditions for fixing cells or nuclei with formaldehyde to allow for long-term stable storage. We found that the buffer used for fixation and the choice of isolating nuclei before or after fixation presented choices between complexity and specificity. In the current study, we chose a fixation protocol that increased complexity/sensitivity at the expense of specificity, however, this can be decided by end users of the protocol.

Materials and Methods

Cell Culture

GM12878 cells were cultured and maintained in RPMI 1640 medium (Thermo Fisher Scientific cat. no. 11875-093) with 15% FBS (Thermo Fisher cat. no. SH30071.03) and 1% Pen-strep (Thermo Fisher cat. no 15140122). They were counted and split at 300,000 cells/ml three times a week. CH12-LX murine cell line was gifted by Michael Snyder lab in Stanford. The cells were cultured in RPMI 1640 medium with 10% FBS, 1% Pen-strep (Penicillin and Streptomycin) and 1×10\hat{ }5M B-ME. They were counted and maintained at a density of 1×10\hat{ }5 cells/ml, splitting three times a week to maintain cell concentration. Both cell lines were incubated at 37° C. with 5% CO2.

Nuclei Isolation and Fixation from Cell Lines

For suspension cells, obtain between ˜10-100 million cells and pellet cells by spinning at 500×g for 5 min at room temperature. Aspirate supernatant and resuspend pellet in 1 ml Omni-ATAC lysis buffer (10 mM NaCl, 3 mM MgCl2, 10 mM Tris-HCl pH 7.4, 0.1% NP40, 0.1% Tween 20 and 0.01% Digitonin) and incubate on ice for 3 min. Add 5 ml of 10 mM NaCl, 3 mM MgCl2, 10 mM Tris-HCl pH 7.4 with 0.1% Tween 20 and pellet nuclei for 5 min at 500×g at 4° C. Aspirate supernatant and resuspend nuclei in 5 ml 1×DPBS (Thermo Fisher cat. no. 14190144). To crosslink the nuclei, add 140 ul of 37% formaldehyde with methanol (VWR cat. no. MK501602) in one shot at a final concentration of 1%. Incubate fixation mixture at room temperature for 10 minute inverting every 1-2 min. To quench the crosslinking reaction, add 250 ul of 2.5 M glycine and incubate at room temperature for 5 minutes and then on ice for 15 minutes to stop cross-linking completely. Take 20 ul of the quenched crosslinked mixture to 20 ul of trypan blue for counting. Spin crosslinked nuclei at 500×g for 5 minutes at 4° C. and aspirate the supernatant. Resuspend fixed nuclei in appropriate amount of freezing buffer (50 mM Tris at pH 8.0, 25% glycerol, 5 mM Mg(OAc)2, 0.1 mM EDTA, 5 mM DTT (Sigma-Aldrich cat. no. 646563-10×0.5 ml), 1× protease inhibitor cocktail (Sigma-Aldrich cat. No. P8340) to obtain 2 million nuclei per 1 ml aliquot, snap freeze in liquid nitrogen and store in −80° C.

Tissue Procurement and Storage

Tissue of interest is isolated and rinsed in 1×HBSS (with Ca. and Mg.) then blotted dry on a semi-damp gauze. Place dried tissue on heavy duty foil or in cryotube and snap freeze tissue using liquid nitrogen. Store frozen tissues at −80° C.

Nuclei Isolation and Fixation of Frozen Fetal Tissues

On day of pulverization, pre-cool pre-labeled tubes and hammer on dry ice with a cloth towel between the dry ice and metal. Create a “padding” by taking an 18″×18″ heavy duty foil, fold in half twice creating a rectangle. Fold twice more to create a square. Place frozen tissue inside the foil “padding” then place tissue in foil padding inside a pre-chilled 4 mm plastic bag to prevent tissue from falling out onto the dry ice in case the foil rupture. Chill this tissue packet between 2 slabs of dry ice. Using the pre-chilled hammer, manually pulverize the tissue inside the packet; 3 to 5 impacts avoiding a grinding motion before taking a break to avoid heating the sample. Chill the hammer and repeat pulverization as needed until tissue is uniform. Aliquot pulverized tissue into pre-labeled and pre-chilled 1.5 ml LoBind and nuclease-free snap cap 1.5 ml tubes (Eppendorf cat. no. 022431021). Aliquots of powdered tissues can be stored at −80° C. until further processing.

On day of nuclei isolation, add lysis buffer directly to tube or pour out the frozen aliquot into a 60 mm dish with cell lysis buffer and further mince with a blade. As long as the aliquot has not thawed at some point in storage, the powdered tissue aliquot should easily slide out of the storage tube without sample loss. We estimate ˜20,000 cells per mg of original tissue weight and performance may vary from tissue to tissue. Resuspend pulverized tissue in lml Omni lysis (RSB+0.1% Tween+0.1% NP-40 and 0.01% Digitonin) then transfer to a 15 ml falcon tube. Incubate nuclei for 3 minutes on ice then add 5 ml RSB+0.1% Tween 20. Centrifuge the nuclei at 500×g for 5 minutes at 4° C. Aspirate supernatant and resuspend in 5 ml 1×DPBS. Pass through nuclei in 1×DPBS into a 100 micron cell strainer (VWR cat. no. 10199-658) to remove tissue clumps. In the fume hood, crosslink the nuclei by adding 140 uL of 37% formaldehyde with methanol in one shot for 1% final concentration and mixing quickly by inverting the tube several times. Incubate at room temperature for exactly 10 minutes with gently inversion of the tube every 1-2 min. Add 250 uL of 2.5M glycine (freshly-made, filter-sterilized) to quench the cross-linking reaction, mix well by inverting the tube several times. Incubate for 5 minutes at room temperature, then on ice for 15 minutes to stop the cross-linking completely. Count nuclei using hemocytometer to know final volume of freezing buffer to add, the goal is to freeze ˜1-2 million nuclei/tube. Centrifuge the cross-linked nuclei at 500×g for 5 minutes at 4° C., aspirate the supernatant and resuspend pellet in 1-10 ml of freezing buffer supplemented with 1× protease inhibitors and 5 mM DTT. Snap-freeze nuclei in liquid nitrogen and store nuclei at −80° C.

Sci-ATAC-Seq3 Sample Processing (Library Construction and Qc)

Take frozen fixed nuclei out of the −80° C. and place on a bed of dry ice. Thaw nuclei in 37° C. water bath until thawed (˜30 sec-1 min) and transfer nuclei into a 15 ml falcon tube. Pellet nuclei at 500×g for 5 minutes at 4° C. Aspirate supernatant without disturbing the pellet and resuspend pellet in 200 ul of Omni lysis buffer then incubate on ice for 3 minutes. Wash out lysis buffer with 1 ml ATAC-RSB with 0.1% Tween 20 and gently invert tube 3 times to mix. Count nuclei by taking 20 ul of nuclei and 20 ul of Trypan blue. While counting, keep nuclei on ice whenever possible from here on. For 3 level indexing experiments at 384\hat{ }3, nuclei input number is 4.8 million @ 50,000 nuclei per well per tissue or sample spread across 96 reactions. Pellet nuclei and resuspend in pre-made tagmentation reaction master mix (Nextera T D buffer, 1×DPBS, 0.1% Digitonin, 0.1% Tween 20, and water). Aliquot 47.5 ul of nuclei in tagmentation mix using wide bore tip (Rainin Instrument Co cat. no.30389249) across a LoBind 96 well plate (Eppendorf cat. No. 30129512). Add 2.5 ul of Nextera v2 enzyme (Illumina Inc cat. no. FC-121-1031) per well, seal plate with adhesive tape and spin at 500×g for 30 sec. Incubate plate at 55° C. for 30 minutes to tagment DNA. Tagmentation reactions were stopped by adding 50 ul of stop reaction mixture (40 mM EDTA with 1 mM Spermidine) then incubated at 37° C. for 15 min. Using wide-bore tips, tagmented nuclei were pooled and pelleted at 500×g for 5 minutes at 4° C. and then washed with ATAC-RSB with 0.1% Tween 20. Pellet nuclei in 500×g for 5 minutes at 4° C., aspirate supernatant and resuspend in 384 ul ATAC-RSB with 0.1% Tween 20. Create a PNK reaction master mix (1× PNK buffer (NEB cat. no. M0201L), 1 mM rATP (NEB cat. no. P0756S), water and T4 Polynucleotide Kinase (NEB cat. no. M0201L) and add to nuclei. Aliquot 5 ul of PNK reaction mix to four LoBind 96 well plates, seal with adhesive tape and spin at 500×g for 5 minutes at 4° C. PNK reaction were incubated at 37° C. for 30 minutes. Directly add 13.8 ul of ligation master mix (1×T7 ligase buffer (NEB, cat. no. M0318L), 9 uM N5_splint (IDT), water and 2.5 ul T7 DNA ligase enzyme (NEB cat. no. M0318L) to the PNK reaction. Using a multi-channel or a 96 head dispenser (Liquidator, cat. No. 17010335), add 1.2 ul of 50 uM N5_oligo (IDT) to each well across four 96 well plates. Seal with adhesive tape and spin at 500×g for 30 seconds then incubate at 25° C. for 1 hour. After first round of ligation, add 20 ul of 40 mM EDTA with 1 mM Spermidine to stop ligation reaction and incubate at 37° C. for 15 minutes. Using wide bore tips, pool each well in a trough and transfer into a 50 ml falcon tube. Pellet nuclei at 500×g for 5 minutes at 4° C., aspirate supernatant and resuspend nuclei in 1 ml of ATAC-RSB with 0.1% Tween 20 to wash any residual ligation reaction mix. Pellet nuclei at 500×g for 5 minutes at 4° C. and aspirate supernatant without disturbing the pellet. Create N7 ligation master mix (1×T7 ligase buffer, 9 uM N7_splint (IDT), water and T7 DNA ligase) and resuspend the nuclei with the ligation master mix. Transfer nuclei suspended in master mix into a trough and using wide bore tips, aliquot 18.8 ul of ligation master mix into four 96 well LoBind plates then add 1.2 ul of 50 uM N7_oligo (IDT) to each well across four 96 well plates. Seal plates with adhesive tape and spin at 500×g for 30 seconds then incubate at 25° C. for 1 hour then stop ligation by adding 20 ul of 40 mM EDTA and I mM Spermidine and incubate at 37° C. for 15 minutes. Pool wells in a trough using wide bore tips and then transfer into a 50 ml falcon tube. Pellet nuclei at 500×g for 5 minutes at 4° C., aspirate supernatant and resuspend nuclei in 2 ml of Qiagen EB buffer (Qiagen cat. no. 19086). Take 20 ul of resuspended nuclei and 20 ul of trypan blue to count nuclei. Dilute nuclei to 100-300 nuclei per ul and aliquot 10 ul per well into four 96 well LoBind plates. To reverse crosslink the nuclei, make a reverse crosslink master mix of EB buffer, Proteinase k (Qiagen, cat. no. 19133) and 1% SDS; 1 ul/0.5 ul/0.5 ul respectively per well) and add 2 ul to each well of nuclei. Seal with adhesive tape, spin at 500×g for 30 seconds and incubate at 65° C. for 16 hours. We performed a test PCR amplification and monitored the reaction with SYBR green on several wells of a plate to determine optimal cycle number. Based on the test PCR result, we amplified the rest of the reverse cross-linked plates with 7.5 ul NPM, 0.5 ul BSA (NEB, cat. no. B9000S), 1.25 ul indexed P5_10 uM (IDT), 1.25 indexed P7_10 uM (IDT) and water per well. Depending on the batch of tissues and nuclei recovery after two rounds of ligation, 11-13 cycles is typical in our hands. Cycling conditions were as follows: 72° C. 3 min, 98° C. 30 sec, 11-13 cycles (98° C. 10 sec, 63° C. 30 sec, 72° C. 1 min) and hold at 10° C. Amplification product from a 96-well plate were pooled in a trough and purified using Zymo Clean & Concentrate-5 (Zymo Research cat. no. D4014) following manufacturer's specifications and split across 4 columns. Elute each column in 25 ul EB buffer and then combined into 1 tube. 100 ul of AMPure bead (Agencourt, cat. no. A63882) were added to purified PCR product to further get rid of any residual primer dimers and follow manufacturer's purification processes. Elute final libraries off of beads in 25 ul Qiagen EB buffer. Quantify final library using D5000 screentape (Agilent cat. no. 5067-5588 screentape, 5067-5589 reagents) Agilent 4200 Tapestation System establishing a 200-1000 base pair window to determine nM concentration of fragments that will cluster well during sequencing. A 2 nM pool were created from equimolar pooling and sequenced at 1.8 pM loading concentration with NextSeq high output 150 cycle kit (Illumina cat. no. 20024904) with custom recipe and primers.

Data Processing for Method Development

Data processing for the barnyard experiments conducted to develop sci-ATAC-seq3 was done as previously described. Briefly, BCL files were converted to fastq files with bcl2fastq v2.16 (Illumina). Each read was associated with a cell barcode made up of 4 components: on the P5 end of the molecule there was a row address for tagmentation and for PCR added and on the P7 end of the molecule there was a column address for tagmentation and PCR added. To correct for errors in these barcodes, we broke them into their 4 constituent parts and corrected them to the closest barcode within an edit distance of 2 so long as this correction was unambiguous at the required edit distance. If any of the four barcodes could not be corrected to a known barcode, the corresponding read pair was dropped. Reads were then trimmed with Trimmomatic using option ‘ILLUMINACLIP:{adapters_path}:2:30:10:1:true TRAILING:3 SLIDINGWINDOW:4:10 MINLEN:20’. Trimmed reads were mapped to a hybrid human/mouse (hg19/mm9) genome using bowtie2 with options ‘−X 2000-3 1’. Subsequently reads not mapping in proper pairs to the genome with a quality of at least 10 were filtered out with samtools using options ‘43-F12-q10’ and only reads mapping to the autosomes or sex chromosomes were retained for downstream analysis. Reads were deduplicated for each cell barcode using a custom script. Note that unlike the pipeline for the tissues (discussed below), read pairs were not maintained in deduplicating.

Data Processing for Tissue Samples

Methods for processing sequencing data from the tissue samples closely follows the methods used in closely as well, albeit with numerous optimizations to scale to larger datasets, but we include a description here for convenience. BCL files were converted to fastq files using bcl2fastq v2.20 (Illumina). Reads with corrected barcodes contained in the read name were written out to a separate R1/R2 file for each of the samples in our dataset. Note that a mapping of all mismatches to the set of known barcodes was precomputed (feasible because of the short length and relatively small number of barcodes), the correction script was run using a pypy (an alternate to the cpython interpreter that is much faster for this particular task), and we parallelized this computation over different lanes of the sequencing run, which collectively improved runtime markedly over our previous method.

We next trimmed low quality bases/adapter sequences from the 3′ end with Trimmomatic using the options ILLUMINACLIP:{adapters_path} TRAILING:3 SLIDINGWINDOW:4:10 MINLEN:20 and then mapped the trimmed reads to the hg19 reference genome using bowtie2 with ‘-X 2000 3 1’ as options and then filtered out read pairs that did not map uniquely to autosomes or sex chromosomes with a mapping quality of at least 10 using Samtools—samtools view -L (whitelist of chromosomes) -f3 -F12 -q10 -bS. The resulting BAM files were sorted, the aligned reads for each sample were merged using sambabamba, and the resulting BAM files were indexed. This process was parallelized across samples/lanes where possible while also providing trimmomatic/bowtie2/sambabamba will multiple threads per process to improve runtime.

We subsequently identified PCR duplicates within cells by identifying the unique set of fragment endpoints within each cell. In our previous work, the resulting deduplicated BAM file did not always maintain the proper read name between the read pairs written out in the deduplicated BAM file (it randomly chose a representative read for R1 and R2 independently for each unique fragment), which had caused compatibility issues with some tools such as SnapATAC (github.com/r3fang/SnapATAC). We corrected this issue and also implemented writing of 1) a BED file of fragment endpoints for each cell and 2) a file closely mirroring the fragments.tsv.gz file provided by 10× Genomics for their scATAC Solution.

Within each sample, the BED file of unique fragment endpoints for each cell was used for peak calling in each sample via MACS2—macs2 callpeak -t (bed) -f BED -g hs --nomodel --shift -100 --extsize 200 --keep-dup all --call-summits -n {sample_name} -o {output_dir}. The resulting {outdir}/{sample_name}_peaks.narrowPeak file was sorted and output as a BED File. Peak calls from all samples included in downstream analysis (additionally excluding our standards) were merged using bedtools to form a master set of peaks. We note that, as we have described previously, the use of BED files for peak calling here is intentional and bipasses the behavior of macs2 on BAM inputs. MACS2, given a BAM file as input, will either discard one of the read pairs which using R1/R2 independently (effectively downsampling the input data) or use the entire insert when computing coverage if explicitly specifying that the BAM file is paired-end (we do not want to compute coverage along the entire insert, just the endpoints). Using a BED file allows use of all data and calculation of coverage only using a window around the molecule endpoints.

For each sample, we additionally created sparse matrices counting 1) reads falling within the master set of peaks, 2) reads falling within gene bodies extended by 2 kb upstream, and 5 kb windows of the genome. We also additionally tabulated the total number of reads from each cell coming from annotated TSSs (+/−1 kb around each TSS), ENCODE blacklist regions, and our set of merged peaks for QC purposes.

We also constructed a peak by motif matrix using the method employed in the 10× genomics scATAC pipeline (see support.10×genomics.com/single-cell-atac/software/pipelines/latest/algorithms/overview). Briefly, the method from 10× calculates the GC % distribution of peaks and bin peaks into equal quantile ranges of GC content such that motif occurrences can be discovered separately within each bin. The MOODS package is used to identify motif occurrences for motifs in the JASPAR motif database at a p-value threshold of 1E-7 and background nucleotide composition matched to the respective GC bin to mitigate GC bias. These hits are used to construct a motif by peak matrix which can be used to compute matrices of motif by cell counts in downstream analyses. This matrix is binarized such that only one instance of a motif can be counted per peak.

Cell barcodes were separated from the distribution of background barcodes using a modified version of the method employed by the 10× genomics scATAC pipeline (see link above). Briefly, we fit a mixture of two negative binomials (noise vs. signal). In place of the method used by 10× to establish an initial threshold between these two distributions, we apply k-means clustering to the log scaled total fragment count distribution and take the maximum value of the cluster with lower average total counts as the initial threshold. This initial threshold is used to determine the starting parameterization for the two distributions using maximum likelihood estimates and is further refined via an expectation maximization approach. As noted by 10×, this fit can be improved via applying a left-shift to the count distribution. Unlike the 10× method, we determine this shift by trying several shifts from 2 to 12 and taking the mixture model with the best goodness of fit. Finally, in contrast to the 10× approach, we apply this method to the total fragment count distribution, not the distribution of counts within called peaks. The final threshold chosen was the minimum count that both yields an odds ratio (in favor of signal) of 20 or higher and would remove at least 0.5% of the signal distribution as estimated from the signal distribution's CDF (we found that this second criteria prevented fits with thresholds that appeared to be too lax otherwise).

Cell-Level QC, Dimensionality Reduction, and Clustering

For each cell, we tabulated the total unique reads and the total number of unique reads falling around TSSs (+/1 kb), in peaks, and in ENCODE blacklist regions as mentioned above. Using these totals we chose sample-specific cut-offs for the fraction of unique reads in peaks and the fraction of unique reads falling in TSSs via visual inspection of their distributions for each sample, and a global cutoff of 0.5% of unique reads coming from ENCODE blacklist regions. Due to a small number of samples having automated thresholds that were substantially lower than other samples in the dataset, we applied a global threshold of 1000 unique reads per cell (or 500 unique fragments per cell) to raise the automated thresholds for the corresponding samples. We examined nucleosome banding scores, which we developed previously, but did not observe a clear distribution of outliers as we did previously for mouse testes and therefore did not use these scores in QC. Peaks overlapping ENCODE blacklist regions or falling on sex chromosomes were removed prior to downstream steps (the latter to avoid the introduction of potential batch effects between samples of different sexes). We also excluded peaks beyond two standard deviations from the mean of the log scaled counts per peaks distribution to remove peaks with very low counts in the tissue being analyzed.

All downstream steps were performed one tissue at a time by pooling passing cells from all samples of a given tissue.

After filtering, we employed a modified version of the scrublet algorithm in an attempt to remove the cells most likely to be doublets. Briefly, we simulate doublets as sums of randomly chosen cells from the dataset using the peak by cell matrix. We then perform LSI as described below using the matrix of the original cells and simulated doublets. Note that in this step we use an inverse document frequency (IDF) term derived from the original dataset without simulated doublets, similar to how scrublet applies scaling factors from the original dataset for scRNA-seq data. In the resulting 50 dimension space, we find nearest neighbors of each cell and calculate the fraction of simulated doublets in the neighborhood as a doublet score. We exclude the top 10% of cells within each sample with the highest doublet score.

For dimensionality reduction, we initially found that the implementation of Latent Semantic Indexing (LSI; or equivalently Latent Semantic Analysis or LSA) that we have previously described, did not perform well on data collected in this study. We reasoned that this was likely due to sparsity and examined several alternate methods including CisTopic and SnapATAC. Each of these methods initially seemed to perform better than our implementation of LSI. We were initially unsure as to why this would be the case given the underlying similarity of these methods and the nature of our data. We discovered that simply log-scaling the term-frequency term in LSI, which we and many others had not done previously, resulted in very similar performance to the other tools we tested. We suspect this is likely due to the exponential distribution of total counts per cell and the impact of strong outliers on the PCA step of LSI in the absence of log scaling. This is discussed in detail here: andrewjohnhill.com/blog/2019/05/06/dimensionality-reduction-for-scatac-data/. We note that the difference observed with and without log scaling is particularly dramatic with sparse datasets where the range of total counts per cell is large. We also note that other groups have since confirmed our own independent findings that LSI compares favorably to all other existing methods for scATAC dimensionality reduction. We also observed very similar performance when using peaks or 5 kb windows of the genome, so opted to use peaks as we have primarily done in previous work.

To summarize, we performed LSI on the binarized window by cell matrix from all passing cells from each tissue one tissue at a time. We first weighted all the sites for individual cells by log(total number of peaks accessible in cell) (log scaled “term frequency”). We then multiplied these weighted values by log(1+the inverse frequency of each site across all cells), the “inverse document frequency.” We then used singular value decomposition on the TF-IDF matrix to generate a lower dimensional representation of the data (PCA) by only retaining the 2nd through 50th dimensions (because the first dimension tends to be highly correlated with read depth). We then performed L2 normalization on the PCA matrix in an effort to further account for differences in the number of unique fragments per cell. This L2-normalized PCA matrix was used for all downstream steps.

Although we did not observe evidence for substantial batch effects between samples, we applied the Harmony batch correction algorithm on the PCA space to correct batch effects between different samples. We chose Harmony primarily due to the fact that it scaled readily to large datasets and allowed us to use our existing PCA coordinates.

This corrected L2-normalized PCA space was used as input to Louvain clustering and UMAP as implemented in Seurat V3.

Specificity Scores

Any peaks overlapping ENCODE blacklist regions were filtered out prior to specificity score calculations. We calculated specificity scores for each site/cell type pair as previously described.

Motif Enrichments

Any peaks overlapping ENCODE blacklist regions were filtered out prior to motif enrichment calculations. We first obtain a matrix of motif by cell counts by multiplying the corresponding peak by cell matrix (as described above aggregated over all cells in the subset of the data being examined) by the peak by motif matrix. Note that we downsample the dataset such that a maximum of 800 cells per annotation (e.g. cell type) are included to reduce computational cost and reduce overrepresentation of very abundant cell types in computing enrichments in downstream steps. For each annotation we then perform a negative binomial regression using the speedglm package, predicting total motif counts using two input variables—an indicator column for the annotation as the main variable of interest and log(total number of non-zero entries in input peak matrix) for each cell as a covariate. We use the coefficient for the annotation indicator column and the intercept to estimate the fold change of the motif count of the annotation of interest relative to cells from all other annotations—exp(intercept+annotation_coefficient)/exp(intercept). We perform this test for all motifs in all groups and then correct p-values using the Benjamini Hochberg procedure.

### Example 2

A human cell atlas of gene expression during development

Abstract

The emergence and differentiation of cell types during human development is of fundamental interest. We applied an assay for single cell profiling of gene expression based on three-level combinatorial indexing (sci-RNA-seq3) to 121 fetal tissues representing 15 organs, altogether profiling transcription in 4-5 million single cells. From these data, we identify cell types and annotate them with respect to marker genes, expression and regulatory modules. We focus our initial analyses of these data on cell types that span multiple organ systems, e.g., epithelial, endothelial and blood cells. Intriguing observations include organ-specific endothelial specialization, potentially novel sites of fetal erythropoiesis, and potentially novel cell types. Together with the accompanying human cell atlas of chromatin accessibility during development, these data are a rich resource for the exploration of human biology.

Main Text

For several reasons, we set out to generate a human cell atlas of both gene expression and chromatin accessibility using tissues obtained during development. First, genetic disorders, the vast majority of which include a developmental component, account for a grossly disproportionate fraction of pediatric morbidity and mortality. These include thousands of Mendelian disorders, as well as more common conditions (e.g., congenital heart defects, other birth defects, neurodevelopmental disorders, etc.) to which both genetic and non-genetic factors substantially contribute. A reference cell atlas generated from developing tissues could serve as the foundation for a systematic effort to understand the specific molecular and cellular events that give rise to each of these pediatric conditions.

Second, developing tissues afford a much better opportunity to study the in vivo emergence and differentiation of human cell types than adult tissues. Relative to embryonic and fetal tissues, adult tissues are dominated by differentiated cells, and moreover many cell states are simply not represented. Through better resolution of in vivo developmental trajectories, a single cell atlas generated from developing tissues could broadly inform our basic understanding of in vivo human biology, as well as strategies for cell reprogramming and cell therapy.

Third, although pioneering cell atlases have already been reported for many adult human organs, the independent nature of these studies makes it difficult to investigate differences amongst cell types appearing in different tissues, e.g., epithelial, endothelial and blood cells. Specifically, comparisons based on existing data are challenged by sample processing and technology platform differences between the groups generating organ-specific cell atlases.

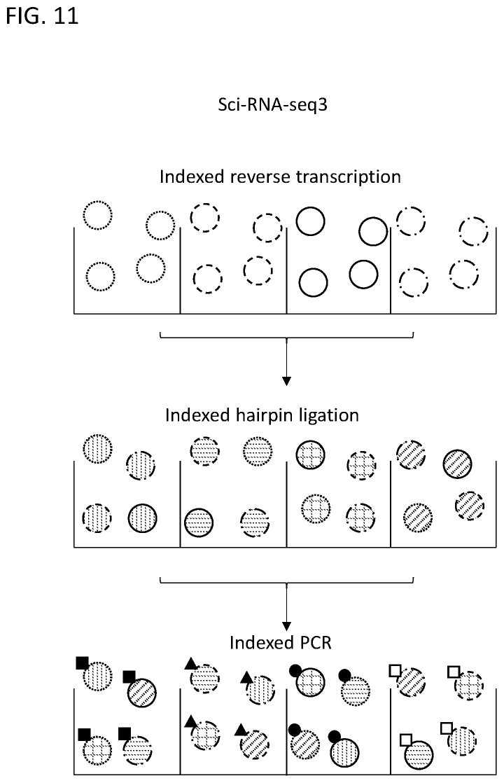

Towards a human cell atlas of gene expression, we applied our recently developed assay for single cell RNA-seq based on three-level combinatorial indexing (sci-RNA-seq3) to 121 fetal tissues representing 15 organs, altogether profiling gene expression in nearly 5 million cells (FIG. 11). In Example 1, we describe profiling of chromatin accessibility in 1.6 million cells from the same organs, based on an overlapping set of samples. The profiled organs span diverse systems; most notably absent are bone marrow, bone, gonads, and skin.

Tissues were obtained from 28 fetuses ranging from 72 to 129 days in gestational age. In brief, these were flash frozen, pulverized, and the resulting powder split for different assays. For sci-RNA-seq3, nuclei were extracted directly from cold, lysed powder and then fixed with paraformaldehyde. For renal and digestive organs where RNases and proteases are abundant, we used paraformaldehyde-fixed cells rather than nuclei, which increased cell and mRNA recovery. For each experiment, nuclei or cells from a given tissue were deposited to different wells, such that the first index of sci-RNA-seq3 protocol also identified the source. As a batch control for experiments on nuclei, we spiked a mixture of human HEK293T and mouse NIH/3T3 nuclei, or nuclei from a common ‘sentinel’ tissue (also used for sci-ATAC-seq3 experiments), into one or several wells. As a batch control for experiments on cells, we spiked cells derived from a common pancreatic tissue (for which nuclei were also profiled) into one or several wells.

We sequenced sci-RNA-seq3 libraries from 7 experiments across 7 Illumina NovaSeq runs, altogether generating 68.6 billion reads. Processing data as previously described, we recovered 4,979,593 single cell gene expression profiles (UMI>250). Single cell transcriptomes from human-mouse control wells were overwhelmingly species-coherent (˜5% collisions). Uniform manifold approximation and projection (UMAP) of nuclei or cells from the sentinel tissues indicated that cell type differences dominated over any inter-experimental batch effects. Integrated analysis using seurat of nuclei and cells corresponding to the common pancreatic tissue also resulted in highly overlapping distributions.

We profiled a median of 72,241 cells or nuclei per organ (max 2,005,512 (cerebrum); min 12,611 (thymus)). Despite relatively shallow sequencing (˜14,000 raw reads per cell) compared to other large-scale single cell RNA-seq atlases, we recovered a comparable number of UMIs per cell or nucleus (median 863 UMIs and 525 genes). As expected, nuclei exhibited a higher proportion of UMIs mapping to introns than cells (56% for nuclei; 45% for cells; p<2.2e-16, two-sided Wilcoxon rank sum test). We henceforth use ‘cells’ to refer to both cells and nuclei unless otherwise stated.

Tissues were readily identified as deriving from a male (n=14) or female (n=14) by sex-specific gene expression. Each of the 15 organs was represented by multiple samples (median 8) including at least two of each sex, and a range of gestational ages. UMAP visualization of ‘pseudo-bulk’ transcriptomes of each tissue clustered by organ rather than individual or experiment. About half of expressed, protein-coding transcripts were differentially expressed across this set of pseudo-bulk transcriptomes (11,766 of 20,033; FDR of 5%).

We applied scrublet to detect 6.4% likely doublet cells, corresponding to a doublet estimate of 12.6% including both within-cluster and between-cluster doublets. We then applied a strategy that we previously developed for our 2 million cell mouse atlas of organogenesis (MOCA) to remove low-quality cells, doublet-enriched clusters and the spiked-in HEK293T and NIH/3T3 cells. All analyses described below are based on the 4,062,980 human single cell gene expression profiles, derived from 112 fetal tissues, that remained after this filtering step.

Identification of 77 Main Cell Types

After filtering against low-quality cells and doublet-enriched clusters, 4 million single cell gene expression profiles were subjected to UMAP visualization and Louvain clustering with Monocle 3 on a per-organ basis. Altogether, we initially identified and annotated 172 cell types, based on cell type-specific markers from the literature. Collapsing common annotations across tissues, these reduced to 77 main cell types, 54 of which were observed in only a single organ (e.g. Purkinje neurons in the cerebellum), and 23 in multiple organs (e.g. vascular endothelial cells in every organ). These 77 main cell types contained a median of 4,829 cells, and ranged from 1,258,818 cells (excitatory neurons in the cerebrum) to only 68 cells (SLC26A4_PAEP positive cells in the adrenal). Each main cell type was contributed to by multiple individuals (median 9). We recovered nearly all main cell types identified by previous atlasing efforts directed at the same organs, despite differences with respect to species, stage of development and technology. We identified a median of 12 main cell types per organ, ranging from 5 (thymus) to 16 (eye, heart and stomach). We did not observe a correlation between the number of profiled cells and the number of identified cell types (p=−0.10, p=0.74).

On average, we identified 11 marker genes per main cell type (min 0, max 294; defined as differentially expressed genes with at least 5-fold difference between first and second ranked cell type with respect to expression; FDR of 5%. There were several cell types that lacked marker genes at this threshold due to similar cell types in other organs (e.g. ENS glia and Schwann cells). For that reason, we also report sets of “within-tissue marker genes” determined by the same procedure but on an organ-by-organ basis (average 147 markers per cell type; min 12, max 778.

Although canonical markers were generally observed and indeed were critical for our annotation process, to our knowledge, the vast majority of observed markers are novel. For example, OLR1, SIGLEC10 and noncoding RNA RP11-480C22.1 are amongst the strongest markers of microglia, together with more established microglial markers such as CLEC7A, TLR7, and CCL3). As anticipated given that these tissues are actively undergoing development, many of the 77 main cell types include states progressing from precursors to one or several terminally differentiated cell types. For example, cerebral excitatory neurons exhibit a continuous trajectory from PAX6+neuronal progenitors to NEUROD6+ differentiating neurons to SLC17A7+ mature neurons. In the liver, hepatic progenitors (DLK1+, KRT8+, KRT18+) exhibit a continuous trajectory to functional hepatoblasts (SLC22A25+, ACSS2+, ASS1+). In contrast with mouse organogenesis, wherein the maturation of the transcriptional program is tightly coupled to developmental time, cell state trajectories were inconsistently correlated with estimated gestational ages in these human data. The simplest explanation is that gene expression is markedly more dynamic during earlier stages of development, i.e., organogenesis vs. fetal development. However, it is also possible that non-uniform representation and inaccuracies in estimated gestational ages confound our resolution.

In addition to these manual annotations of cell types, we also generated semi-automated classifiers for each organ, as well as a global classifier, using Garnett. The Garnett classifiers were generated agnostic of clustering using marker genes separately compiled from the literature. Classifications by Garnett were highly concordant with manual classifications, e.g. 88% of cells were concordant in pancreas (cluster-extended; 5% not concordant, 7% unclassified). Using the Garnett models trained on this human cell atlas, we were also able to accurately classify cell types from other single cell datasets, including data from different methods as well as from adult organs. For example, we applied the Garnett classifier for pancreas to inDrop single cell RNA-seq data and found that the model correctly annotated 82% of the cells (cluster-extended; 11% incorrect, 8% unclassified). These Garnett models are posted to our website where they can broadly be used for the automated classification of single cell data from diverse organs.

Integration Across Tissues and Investigation of Unexpected Cell Types

We next sought to integrate data and compare cell types across all 15 organs. To mitigate the effects of gross differences in the numbers of cells sampled per organ and/or cell type, we randomly sampled 5,000 cells per cell type per organ (or in cases where less than 5,000 cells of a given cell type were represented in a given organ, all cells were taken), and performed UMAP visualization based on the top differentially expressed genes across cell types within each organ. As expected, cell types represented in multiple organs were generally clustered together, e.g., stromal cells, lymphatic endothelial cells and mesothelial cells. Developmentally related cell types generally colocalized as well, e.g., diverse blood cells, PNS neurons, mesenchyme.

We exploited this global UMAP to shed light on cell types that were not clearly annotatable or expected in an organ in which they were initially observed. In many cases, colocalization with an annotated cell type in the global UMAP shed light on their identity. For example, we observe cells in the lung and adrenal gland that are highly correlated with trophoblast giant cells from the placenta (e.g. expressing high levels of placental lactogen, chorionic gonadotropin, and aromatase), suggesting that these are trophoblasts that have entered fetal circulation (CSH1_CSH2_positive cells). More surprisingly, we observe cells in the placenta and spleen that are highly correlated with hepatoblasts (e.g., expressing high levels of serum albumin, alpha fetoprotein, and apolipoproteins) (AFP_ALB_positive cells).

In the heart, we observed three cell types that were not expected based on previous atlasing efforts. The first of these (SATB2_LRRC7 positive neurons) are strongly correlated with CNS excitatory neurons, and express markers including SATB2, PTPRD and DAB1. To our knowledge, this is an unexpected observation. Although we cannot fully rule out contamination from another tissue, we observe these cells in every heart sampled (n=9) at a consistent proportion (range), and we furthermore do not observe other CNS-like cell types in the heart. The other two are highly correlated with cardiomyocytes but express distinct programs that may reflect specialized roles. Specifically, the ELF3_AGBL2 positive cardiomyocyte-like cells specifically express many genes associated with pulmonary alveolar surfactant secreting cells, including pulmonary secretory protein 1 (SCGB3A2), pulmonary surfactant-associated protein B (SFTPB) and pulmonary surfactant-associated protein C (SFTPC), while the CLC_IL5RA positive cardiomyocyte-like cells specifically express immune cell-related receptors, including interleukin 5 receptor Subunit Alpha (IL5RA) and hematopoietic-specific transmembrane protein 4 (MS4A3).

Characterization of cell type-specific gene regulatory networks and pathways.

We next investigated the cell type-specific expression of surface and secreted protein coding genes critical for regulating cell-cell or cell-environmental interactions. Most surface proteins (4,565 of 5,480) and most secreted proteins (2,491 of 2,933) were differentially expressed across the 77 main cell types (FDR of 0.05). For example, microglia specifically express sialic acid-binding immunoglobulin-like lectin 8 (SIGLEC8) and the oxidized LDL endocytosis receptor (OLR1), both associated with Alzheimer's disease; endothelial cells specifically express roundabout guidance receptor 4 (ROBO4) and endothelial cell adhesion molecule (ESAM), both involved in angiogenesis and vascular patterning. Similarly, different neurons were marked by distinct cell surface transporters. For example, in the cerebellum, we observe specific expression of glycine neurotransmitter transporter SLC6A5 in inhibitory interneurons, excitatory amino-acid transporter SLC1A6 in Purkinje neurons, potassium channel KCNK9 in granule neurons, and sodium/potassium/calcium exchanger SLC24A4 in SLC24A4_PEX5L positive inhibitory neurons. There are similarly myriad examples of cell type-specific expression of secreted proteins. A particularly intriguing example is an unexpected cell type in the spleen (STC2_TLX1 positive cells) that specifically expresses the glycoprotein S7C2, as well as the TFs TLX1 and NKX2-3, all associated with mesenchymal precursor or stem cells.

Noncoding RNAs have been demonstrated to play an important role in normal development as well as disease. In these data, 3,130 of 10,695 noncoding RNAs were differentially expressed across the 77 main cell types (FDR of 0.05), e.g., ncRNAs highly specific to microglia (RP11-489018.1, RP11-480C22.1, RP11-10H3.1) or endothelial cells (AC011526.1, RP11-554D15.1, CTD-3179P9.1). Although the biological significance of such cell type-specific ncRNAs remains unclear, it is notable that their patterns of expression were sufficient to separate the 77 main cell types into developmentally coherent groups.

The vast majority of transcription factors (TFs) were also differentially expressed across the 77 main cell types (1,715 of 1,984; FDR of 0.05). Many of the most specific TFs for each cell type were consistent with expectations, e.g. RBPJL for acinar cells, OLG1 and OLG2 for oligodendrocytes and PAX7 for satellite cells. In other cases, cell type-specific TFs informed our consideration of unexpected cell types, e.g. a stromal cell type observed in the pancreas and characterized by the expression of lymphoid chemokines (CCL19_CCL21 positive cells) specifically expresses TFs related to immune activation.

We sought to directly predict TF-target gene interactions through gene expression data. In brief, candidate interactions were identified by covariance between TF expression and target gene expression across the full dataset. These interactions were further filtered by ChIP-seq binding and motif enrichment analysis (Methods). 56,272 candidate TF-target gene links remained, involving 706 TFs and 12,868 target genes. 220 of these 706 TF-linked gene sets showed enrichment of the corresponding TF (FDR of 0.05) in a manually curated database of TF networks (TRRUST) or Enrichr TF-gene networks (e.g. the top enriched TRRUST TF for 330 genes that we link to E2F1 is E2F1, adjusted p-value=2.2e-14; the top Enrichr TF for 1,219 genes that we link to FLI1 is FLI1, adjusted p-value=5.6e-122). When we permuted the target genes assigned to these 706 TFs and repeat the analysis, none of the TF-linked gene sets are significantly enriched for the corresponding TF at the same threshold.

Characterization of Blood Lineage Development Across Organs

The nature of this dataset creates an opportunity to investigate organ-specific differences in gene expression within broadly appearing cell types, e.g. blood cells, endothelial and epithelial cells. As a first such analysis, we reclustered 103,766 cells, derived from all organs, that corresponded to hematopoietic cell types. We then performed Louvain clustering and further annotated fine-grained immune cell types, based on published gene markers, in some cases identifying very rare cell types. For example, myeloid cells separate into microglia, macrophages and diverse dendritic cell subtypes (CD1C+, S100A9+, CLEC9A+ and pDCs). The microglial cluster primarily derives from the cerebrum and cerebellum, and is well separated from macrophages, consistent with their distinct developmental origins. Lymphoid cells clustered into several groups including B cells, NK cells, ILC 3 cells, and T cells (the latter including the thymopoiesis trajectory). We also recovered very rare cell types such as plasma cells (139 cells, which is 0.1% of all blood cells or 0.003% of the full dataset; mostly in placenta) and TRAF1+ APCs (189 cells, which is 0.2% of all blood cells or 0.005% of the full dataset; mostly in thymus and heart).

Although gene expression markers for different immune cell types have been extensively studied, these may be limited by their definition via a restricted set of organs or cell types. Indeed, here we found that many conventional immune cell markers were expressed in multiple cell types. For example, conventional markers for T cells, were also expressed in macrophages and dendritic cells (CD4) or NK cells (CD8A), consistent with other studies. We computed pan-organ cell type-specific markers across 14 blood cell types. For example, T cells specifically expressed CD8B and CD5 as expected, but also TENM1. ILC 3 cells, whose annotation was based on their expression of RORC and KIT, were more specifically marked by SORCSJ and JMY. These and other pan-organ-defined markers may be useful for labeling and purifying human fetal blood cell types in future studies.

As expected, different organs showed highly varied proportions of blood cells. For example, the liver contained the highest proportion of erythroblasts, consistent with its role as the primary site of fetal erythropoiesis, while T cells were enriched in the thymus and B cells in the spleen. Nearly blood cells recovered from the cerebellum and cerebrum were microglia. Collective analysis also enabled the identification of rare cell populations in specific organs. For example, we identified rare HSCs in the liver, spleen, and thymus, but also in the heart, lung, adrenal, and intestine.

Focusing on erythropoiesis, we observed a continuous trajectory from HSCs to an intermediate cell type, Erythroid-Basophil-Megakaryocyte biased Progenitors (EBMP), which then split to erythroid, basophilic and megakaryocytic trajectories, consistent with a recent study in mouse fetal liver. This consistency was despite differences in species (human vs. mouse), techniques (sci-RNA-seq3 vs. 10×) and organs (pan vs. fetal). With unsupervised clustering and adopting terminology from that study, we further partitioned the continuum of erythroid states into three stages: early erythroid progenitors (EEPs; marked by SLC16A9 and FAM178B), committed erythroid progenitors (CEPs; marked by KIF18B and KIF15), and cells in the erythroid terminal differentiation state (ETDs; marked by TMCC2 and HBB). Early and late stages of megakaryocytic cells were also readily identified. The corresponding dynamics of genome-wide chromatin accessibility in the erythroid lineage is considered further in the companion manuscript.

As expected given their established role in fetal erythropoiesis, a substantial proportion of immune cells in the liver and spleen corresponded to EEPs, CEPs and megakaryocyte progenitors. Surprisingly, we also observed EEPs, CEPs and megakaryocyte progenitors in the adrenal gland, in every sample studied. Because we do not observe cell types that are more common in the liver and spleen, trival contamination during recovery of the in the adrenal gland is an unlikely explanation. Although confirmation by an orthogonal method is required, the result suggests the possibility of the adrenal gland as an additional site of fetal erythropoiesis.

Macrophages are even more widely distributed. We next collated all macrophages, together with microglia from the brain, and subjected them independently to UMAP visualization and Louvain clustering. Microglia were divided into three sub-clusters, one of which, marked by IL1B and TNFRSF10D, likely represents activated microglia involved in inflammatory responses. The other microglial clusters were marked by expression of TMEM119 and CX3CR1 (more common in cerebrum) or PTPRC and CDC14B (more common in cerebellum).

The macrophages outside the brain clustered into three major groups: 1) antigen presenting macrophages, found mostly in GI tract organs (intestine and stomach) and marked by high expression of antigen presenting (HLA-DPB1, HLA-DQA1) and inflammatory activation (AHR) genes; 2) perivascular macrophages, found in most organs, with specific expression of markers such as F13A1 and COLEC12, as well as novel markers such as RNASE1 and LYVEL 3) phagocytic macrophages, enriched in the liver, spleen and adrenal gland, with specific expression of markers such as CD5L, TIMD4 and VCAM1. Phagocytic macrophages are critical for erythrophagocytosis; their observation in the adrenal gland is consistent with its aforementioned potential role as a site of fetal erythropoiesis.

Characterization of Endothelial and Epithelial Cells Across Organs

As a second analysis of a single cell type across many organs, we reclustered cells, derived from all organs, that corresponded to vascular endothelium, lymphatic endothelium, or endocardium. These three groups readily separated from one other, and vascular endothelial cells further clustered, at least to some degree, by organ. That organ-specific differences are more readily detected than differences between arteries, capillaries and veins, is consistent with previous cell atlases of the adult mouse.

Differential expression gene analysis identified 700 markers that are specifically expressed in a subset of endothelial cells (FDR of 0.05, over 2-fold expression difference between first and second ranked cluster). About one-third of these (236 of 700) encoded membrane proteins, many of which appeared to correspond to potential specialized functions. For example, renal endothelial cells specifically expressed acid-sensing ion channel 2 (ASIC2), a mechanosensor involved in myogenic constriction and regulation of blood flow in the kidney. Pulmonary endothelial cells specifically expressed relaxin family peptide receptor 1 (RXFP1), which is involved in endogenous nitric oxide-mediated vascular relaxation in the lung specifically expressed sodium-dependent lysophosphatidylcholine transporter symporter 1 (MFSD2A), which is integrally involved in the establishment and function of the blood brain barrier. The potential regulatory basis for differential gene expression in subsets of endothelium is discussed in the companion paper.

As a third analysis of a broadly distributed cell type, we reclustered epithelial cells, derived from all organs, and subjected these to UMAP visualization. While some epithelial cell types were highly organ-specific, e.g., acinar (pancreas) and alveolar cells (lung), epithelial cells with similar functions generally clustered together. For example, the expression programs of squamous epithelial cells (lung, stomach) are co-clustered with corneal and conjunctival epithelial cells (eye), while PDE1C_ACSM3 positive cells (stomach) co-clustered with intestinal epithelial cells (intestine).

Within epithelial cells, two neuroendocrine cell clusters were identified. The simpler of these corresponded to adrenal chromaffin cells and was marked by the specific expression of HMX1 (NKX-5-3), a TF involved in sympathetic neuron diversification. The other cluster comprised neuroendocrine cells from multiple organs (stomach, intestine, pancreas, lung) and was marked by specific expression of NKX2-2, a TF with a key role in pancreatic islet and enteroendocrine differentiation. We performed further analysis on the latter group, identifying five subsets: 1) pancreatic islet beta cells, marked by insulin expression; 2) pancreatic islet alpha/gamma cells, marked by pancreatic polypeptide and glucagon expression; 3) pancreatic islet delta cells, marked by somatostatin expression; 4) pulmonary neuroendocrine cells (PNECs), marked by expression of ASCL1, a TF with a key role in specifying this lineage in the lung; and 5) enteroendocrine cells. Enteroendocrine cells further comprised several subsets including NEUROG-expressing pancreatic islet epsilon progenitors, TPH1-expressing enterochromaffin cells in both the stomach and intestine, gastrin- or cholecystokinin-expressing G/L/K/I cells. Finally, we observed ghrelin-expressing enteroendocrine progenitors in the stomach and intestine, but also ghrelin-expressing endocrine cells in the developing lung. As the diverse functions of neuroendocrine cells are closely linked with their secreted proteins, we identified 1,086 secreted protein-coding genes differentially expressed across neuroendocrine cells (FDR of 0.05). For example, PNECs showed specific expression of trefoil factor 3, involved in mucosal protection and lung ciliated cell differentiation, gastrin-releasing peptide, which stimulates gastrin release from G cells in the stomach, and SCGB3A2, a surfactant associated with lung development.

As an illustrative example of how these data can be used to explore cell trajectories, we further investigated the path of epithelial cell diversification leading to renal tubule cells. Combining and reclustering ureteric bud metanephric cells, we identified both progenitor and terminal renal epithelial cell types, with differentiation paths that are highly consistent with a recent study of the human fetal kidney. By differential gene expression analysis, we further characterized TFs potentially regulating their specification. For example, nephron progenitors in the metanephric trajectory expressed high levels of mesenchyme and meis homeobox genes (MEOX1, MEIS1, MEIS2), while podocytes specifically expressed MAFB and TCF2/POD1. As another example, HNF4A was specifically expressed in proximal tubule cells; a mutation of this gene causes Fanconi renotubular syndrome, a disease that specifically affects the proximal tubule, and it was recently shown to be required for formation of the proximal tubule in mice.

Comparison of Human and Mouse Developmental Atlases

To investigate the developmental relationships between cell types, we next compared these data to our recent mouse organogenesis cell atlas (MOCA), which profiled 2 million cells from the entire embryo spanning E9.5 to E13.5, an earlier window of mammalian development.

As a first approach, we compared the 77 main human cell types defined here against the developmental trajectories defined by MOCA via a cell type cross-matching method that we previously described. In brief, this method uses non-negative least squares (NNLS) regression to select reciprocal best matched cell type pairs from two data sets. Most human cell types strongly matched to a single major mouse trajectory and sub-trajectory. These generally corresponded to expectation and serve as one form of validation for both sets of annotations. A few discrepancies facilitated important corrections to the MOCA annotations. Many of the human cell types and mouse trajectories that lacked strong matches (combined NNLS regression coefficient<0.6) corresponded to tissues excluded in the other dataset (e.g., mouse placenta; human skin and gonads). Other ambiguities probably follow from the gap between the developmental windows studied (e.g. adrenal cell types), rarity (e.g. bipolar cells) and/or complex relationships between cell types (e.g. fetal cell types that derive from multiple embryonic trajectories).

As a second approach, we sought to directly cluster human and mouse cells together. In brief, we sampled 100,000 mouse embryonic cells from MOCA (randomly) and 65,000 human fetal cells (up to 1,000 cells from each of 77 cell types) and subjected these to Seurat's recently described strategy for integrating cross-species scRNA-seq datasets. The distribution of mouse cells in the resulting UMAP based visualization was strikingly similar to our global analysis of MOCA. Moreover, the cells were largely distributed in a sensical manner with respect to both developmental and temporal relationships instead of spatial organ locations, albeit with some surprises. For example, we observe that: human fetal endothelial, hematopoietic, hepatic, epithelial and mesenchymal cells all mapped to corresponding mouse embryonic trajectories. While the human fetal cerebral and cerebellar neurons overlapped with the mouse embryonic neural tube trajectory, human fetal neural crest derivatives such as ENS neurons, visceral neurons, sympathoblasts and chromaffin cells clustered separately from the corresponding mouse embryonic trajectories, possibly due to excessive differences between the species or developmental stages. As expected, human ENS glia, as well as Schwann cells overlapped with mouse embryonic PNS glia sub-trajectories. Human fetal astrocytes clustered with the mouse embryonic neural epithelial trajectory (mouse astrocytes do not develop till E18.5). Human fetal oligodendrocytes overlap with a rare mouse embryonic sub-trajectory (Pdgfra+ glia) that in retrospect corresponds to oligodendrocyte precursor cells (OPCs; Olig1+, Olig2+, Brinp3+), and calls into question our previous annotation of a different Oligo1+ sub-trajectory as oligodendrocyte precursors.

To visualize more detailed relationships between human fetal and mouse embryonic cells, we applied a similar integrative analysis strategy to extracted human and mouse cells from haematopoietic, endothelial and epithelial trajectories. Data from this fetal human cell atlas readily deconvolute “whole embryo” mouse data into fine-grained functional or spatial groups. For example, subsets of the mouse “white blood cell” trajectory map to specific human blood cell types such as HSCs, microglia, macrophages (liver and spleen), macrophages (other organs) and DCs. These subsets were further validated by the expression of related blood cell markers. Similarly, we observe that related subsets of mouse/human endothelial and epithelial cells to map to one another. This approach may be useful for obtaining the gene expression programs of progenitors of specific lineages at developmental time points that are difficult to access or anatomically resolve. For example, within the mouse cells that we had previously labeled as the foregut epithelial trajectory, we are now able to resolve the likely contributors to the stomach vs. pancreas.

Discussion

The successful development of a functional human fetus is an amazing process, characterized by the process of cell proliferation and differentiation across three key developmental stages.

Following a short (two weeks from fertilization) germinal period with simple cell multiplication and implantation in the uterus, embryogenesis stage continues to gastrulation, neurulation and organogenesis, featured with intense cell differentiation and the generation of internal organ precursors. By the end of the tenth week of gestational age, the embryo has acquired its basic form, called fetus. For the next twenty weeks, different organs continue to grow and mature, with diverse terminal differentiated cell types generated from precursors.

Both germinal and embryogenesis stages have been intensively profiled at single cell resolution in human or model systems (i.e., mouse) with shared early development programs. The late development stage (fetus stage) shows varied development program and length between Homo sapiens and other species. And it has been challenging to obtain a global view of cell dynamics in this stage due to higher organism complexity and technique limitations. Although several single cell studies of fetus development are recently released, they are mostly restricted to a specific organ or cell lineage, and failed to obtain a global view of whole organism development.

Materials and Methods:

Mammalian Cell Culture and Nuclei Extraction

All mammalian cells were cultured at 37° C. with 5% CO₂, and were maintained in high glucose DMEM (Gibco cat. no. 11965) supplemented with 10% FBS and 1×Pen/Strep (Gibco cat. no. 15140122; 100 U/ml penicillin, 100 μg/ml streptomycin). Cells were trypsinized with 0.25% typsin-EDTA (Gibco cat. no. 25200-056) and split 1:10 three times per week.

All cell lines were trypsinized, spun down at 300×g for 5 min (4° C.) and washed once in 1× ice-cold PBS. 5M cells were combined and lysed using 1 mL ice-cold cell lysis buffer (10 mM Tris-HCl, pH 7.4, 10 mM NaCl, 3 mM MgCl2 and 0.1% IGEPAL CA-630 from, modified to also include 1% SUPERase In RNase inhibitor and 1% BSA). The filtered nuclei were then transferred to a new 15 ml tube (Falcon) and pelleted by centrifuge at 500×g for 5 min at 4° C. and washed once with 1 ml ice-cold cell lysis buffer. The nuclei were fixed in 4 ml ice cold 4% paraformaldehyde (EMS) for 15 min on ice. After fixation, the nuclei were washed twice in 1 ml nuclei wash buffer (cell lysis buffer without IGEPAL), and re-suspended in 500 ul nuclei wash buffer. The samples were split to 5 tubes with 100 ul in each tube and flash frozen in liquid nitrogen.

Human Fetal Tissues Preparation and Nuclei Extraction

Human fetal tissues were processed together to reduce batch effects. Each organ was pulverized into tissue powders with hammer (on dry ice) and mixed prior sampling. 0.1-1 g powders were first incubated with 1 mL ice-cold cell lysis buffer (10 mM Tris-HCl, pH 7.4, 10 mM NaCl, 3 mM MgCl2 and 0.1% IGEPAL CA-630 from⁵³, modified to also include 1% SUPERase In and 1% BSA) and then transferred to the top of a 40 μm cell strainer (Falcon). Tissues were homogenized with the rubber tip of a syringe plunger (5 ml, BD) in 4 ml cell lysis buffer. The filtered nuclei were then transferred to a new 15 ml tube (Falcon) and pelleted by centrifuge at 500×g for 5 min and washed once with 1 ml cell lysis buffer. The nuclei were fixed in 5 ml ice cold 4% paraformaldehyde (EMS) for 15 min on ice. After fixation, the nuclei were washed twice in 1 ml nuclei wash buffer (cell lysis buffer without IGEPAL), and re-suspended in 500 μl nuclei wash buffer. The samples were split into two tubes with 250 μl in each tube and flash frozen in liquid nitrogen. For human cell extraction in some organs (kidney, pancreas, intestine, and stomach) and paraformaldehyde fixation.

Sci-RNA-Seq3 Library Preparation and Sequencing

The paraformaldehyde fixed nuclei were processed similarly with the published sci-RNA-seq3 protocol with slight modifications. Briefly, thawed nuclei were permeabilized with 0.2% TritonX-100 (in nuclei wash buffer) for 3 min on ice, and briefly sonicated (Diagenode, 12 sec on low power mode) to reduce nuclei clumping. The nuclei were then washed once with nuclei wash buffer and filtered through 1 ml Flowmi cell strainer (Flowmi). Filtered nuclei were spun down at 500×g for 5 min and resuspended in nuclei wash buffer. Nuclei from each sample were then distributed into several individual wells in four 96-well plates. The links between well id and mouse embryo were recorded for downstream data processing. For each well, 80,000 nuclei (16 μL) were mixed with 8 μl of 25 μM anchored oligo-dT primer (5′-/5Phos/CAGAGCNNNNNNNN[10 bp barcode]TTTTTTTTTTTTTTTTTTTTTTTTTTTTTT-3′ (SEQ ID NO:1), where “N” is any base; IDT) and 2 μL 10 mM dNTP mix (Thermo), denatured at 55° C. for 5 min and immediately placed on ice. 14 μL of first-strand reaction mix, containing 8 μL 5× Superscript IV First-Strand Buffer (Invitrogen), 2 μl 100 mM DTT (Invitrogen), 2 μl SuperScript IV reverse transcriptase (200 U/μl, Invitrogen), 2 μL RNaseOUT Recombinant Ribonuclease Inhibitor (Invitrogen), was then added to each well. Reverse transcription was carried out by incubating plates by gradient temperature (4° C. 2 minutes, 10° C. 2 minutes, 20° C. 2 minutes, 30° C. 2 minutes, 40° C. 2 minutes, 50° C. 2 minutes and 55° C. 10 minutes).

After reverse transcription reaction, 60 μL nuclei dilution buffer (10 mM Tris-HCl, pH 7.4, 10 mM NaCl, 3 mM MgCl2 and 1% BSA) was added into each well. Nuclei from all wells were pooled together and spun down at 500×g for 10 min. Nuclei were then resuspended in nuclei wash buffer and redistributed into another four 96-well plates with each well including 20 μL Quick ligase buffer (NEB), 2 μL Quick DNA ligase (NEB), 10 μL nuclei in nuclei wash buffer, 8 μL barcoded ligation adaptor (100 uM, 5′-GCTCTG[9 bp or 10 bp barcode A]/dideoxyU/ACGACGCTCTTCCGATCT[reverse complement of barcode A]-3′(SEQ ID NO:2)). The ligation reaction was done at 25° C. for 10 min. After ligation reaction, 60 μL nuclei dilution buffer (10 mM Tris-HCl, pH 7.4, 10 mM NaCl, 3 mM MgCl2 and 1% BSA) was added into each well. Nuclei from all wells were pooled together and spun down at 600×g for 10 min.

Nuclei were washed once with nuclei wash buffer and filtered with 1 ml Flowmi cell strainer (Flowmi) once, counted and redistributed into eight 96-well plates with each well including 2,500 nuclei in 5 μL nuclei wash buffer and 3 μL elution buffer (Qiagen). 1.33 μl mRNA Second Strand Synthesis buffer (NEB) and 0.66 μl mRNA Second Strand Synthesis enzyme (NEB) were then added to each well, and second strand synthesis was carried out at 16° C. for 180 min.

For tagmentation, each well was mixed with 11 μL Nextera TD buffer (Illumina) and 1 μL i7 only TDE1 enyzme (62.5 nM, Illumina, diluted in Nextera TD buffer (Illumina)), and then incubated at 55° C. for 5 min to carry out tagmentation. The reaction was then stopped by adding 24 μL DNA binding buffer (Zymo) per well and incubating at room temperature for 5 min. Each well was then purified using 1.5× AMPure XP beads (Beckman Coulter). In the elution step, each well was added with 8 μL nuclease free water, 1 μL 10×USER buffer (NEB), 1 μL USER enzyme (NEB) and incubated at 37° C. for 15 min. Another 6.5 μL elution buffer was added into each well. The AMPure XP beads were removed by magnetic stand and the elution product (16 μL) was transferred into a new 96-well plate.

For PCR amplification, each well (16 μL product) was mixed with 2 μL of 10 μM indexed P5 primer (5′-AATGATACGGCGACCACCGAGATCTACAC[i5]ACACTCTTTCCCTACACGACGC TCTTCCGATCT-3′ (SEQ ID NO:3); IDT), 2 μL of 10 μM P7 primer (5′-CAAGCAGAAGACGGCATACGAGAT[i7]GTCTCGTGGGCTCGG-3′ (SEQ ID NO:4), IDT), and 20 μL NEBNext High-Fidelity 2×PCR Master Mix (NEB). Amplification was carried out using the following program: 72° C. for 5 min, 98° C. for 30 sec, 12-16 cycles of (98° C. for 10 sec, 66° C. for 30 sec, 72° C. for 1 min) and a final 72° C. for 5 min.

After PCR, samples were pooled and purified using 0.8 volumes of AMPure XP beads. Library concentrations were determined by Qubit (Invitrogen) and the libraries were visualized by electrophoresis on a 6% TBE-PAGE gel. All libraries were sequenced on one NovaSeq platform (Illumina) (Read 1: 34 cycles, Read 2: 52 cycles, Index 1: 10 cycles, Index 2: 10 cycles).

For paraformaldehyde fixed cells, they were processed similarly as the fixed nuclei with slight modifications: frozen fixed cells were thawed on 37° C. water bath, spun down at 500×g for 5 min, and incubated with 500 ul PBSR (1×PBS, pH 7.4, 1% BSA, 1% SuperRnaseIn, 1% 10 mM DTT) including 0.2% Triton X-100 for 3 min on ice. Cells were pelleted and resuspended in 500 ul nuclease free water including 1% SuperRnaseIn. 3 ml 0.1N HCl were added into the cells for 5 min incubation on ice (7). 3.5 ml Tris-HCl (pH=8.0) and 35 ul 10% Triton X-100 were added into cells to neutralize HCl. Cells were pelleted and washed with 1 ml PBSR. Cells were pelleted and resuspended in 100 ul PBSI (1×PBS, pH 7.4, 1% BSA, 1% SuperRnaseIn). The following steps were similar with the above sci-RNA-seq3 protocol (with paraformaldehyde fixed nuclei) with slight modifications: (1) We distributed 20,000 fixed cells (instead of 80,000 nuclei) per well for reverse transcription. (2) We replaced all nuclei wash buffer in following steps with PBSI. (3) All nuclei dilution buffer were replaced with PBS+1% BSA.

Processing of Sequencing Reads

Read alignment and gene count matrix generation for the single cell RNA-seq was performed using the pipeline that we developed for sci-RNA-seq3 with minor modifications: base calls were converted to fastq format using Illumina's bcl2fastq/v2.16 and demultiplexed based on PCR i5 and i7 barcodes using maximum likelihood demultiplexing package deML with default settings. Downstream sequence processing and single cell digital expression matrix generation were similar to sci-RNA-seq except that RT index was combined with hairpin adaptor index, and thus the mapped reads were split into constituent cellular indices by demultiplexing reads using both the RT index and ligation index (ED<2, including insertions and deletions). Briefly, demultiplexed reads were filtered based on RT index and ligation index (ED<2, including insertions and deletions) and adaptor clipped using trim_galore/v0.4.1 with default settings. Trimmed reads were mapped to the human reference genome (hg19) for human fetal nuclei, or a chimeric reference genome of human hg19 and mouse mm10 for HEK293T and NIH/3T3 mixed nuclei, using STAR/v 2.5.2b with default settings and gene annotations (GENCODE V19 for human; GENCODE VM11 for mouse). Uniquely mapping reads were extracted, and duplicates were removed using the unique molecular identifier (UMI) sequence (ED<2, including insertions and deletions), reverse transcription (RT) index, hairpin ligation adaptor index and read 2 end-coordinate (i.e. reads with UMI sequence less than 2 edit distance, RT index, ligation adaptor index and tagmentation site were considered duplicates). Finally, mapped reads were split into constituent cellular indices by further demultiplexing reads using the RT index and ligation hairpin (ED<2, including insertions and deletions). For mixed-species experiment, the percentage of uniquely mapping reads for genomes of each species was calculated. Cells with over 85% of UMIs assigned to one species were regarded as species-specific cells, with the remaining cells classified as mixed cells or “collisions”. To generate digital expression matrices, we calculated the number of strand-specific UMIs for each cell mapping to the exonic and intronic regions of each gene with python/v2.7.13 HTseq package⁵⁶. For multi-mapped reads, reads were assigned to the closest gene, except in cases where another intersected gene fell within 100 bp to the end of the closest gene, in which case the read was discarded. For most analyses we included both expected-strand intronic and exonic UMIs in per-gene single-cell expression matrices.

After the single cell gene count matrix was generated, cells with fewer than 250 UMIs were filtered out. Each cell was assigned to its original human fetal sample based on the RT barcode. Reads mapping to each fetus individual were aggregated to generate “bulk RNA-seq”. For sex separation of fetus, we counted reads mapping to a female-specific non-coding RNA (TSIX and XIST) or chrY genes (except genes TBL1Y, RP11-424G14.1, NLGN4Y, AC010084.1, CD24P4, PCDH11Y, and TTTY14, which are detected in both males and females). Fetuses were readily separated into females (more reads mapping to TSIX and MST than chrY genes) and males (more reads mapping to chrY genes than TSIX and MST).

Clustering analysis of whole human fetal samples was done by Monocle 3. Briefly, an aggregated gene expression matrix was constructed as described above for human fetal organs from each individual. Samples with over 5,000 total UMIs were selected. The dimensionality of the data was reduced by PCA (10 components) first on the top 500 most highly dispersed genes and then with UMAP (max_components=2, n_neighbors=10, min_dist=0.5, metric=‘cosine’).

Cell Filtering, Clustering and Marker Gene Identification

For the detection of potential doublet cells, we first split the dataset into subsets for each organ and individual, and then applied the scrublet/v0.1 pipeline to each subset with parameters (min_count=3, min_cells=3, vscore_percentile=85, n_pc=30, expected_doublet_rate=0.06, sim_doublet_ratio=2, n_neighbors=30, scaling_method=‘log’) for doublet score calculation. Cells with doublet score over 0.2 are annotated as detected doublets. We detected 6.4% potential doublet cells in the whole data set, which corresponds to an overall estimated doublet rate of 12.6% (including both within- and between-cluster doublets).

For detection of doublet derived subclusters for cells from each organ, we used an iterative clustering strategy as shown before. Briefly, gene count mapping to sex chromosomes were removed before clustering and dimensionality reduction. Preprocessing steps were similar to the approach used by ref. Briefly, genes with no count were filtered out and each cell was normalized by the total UMI count per cell. The top 1,000 genes with the highest variance were selected and the digital gene expression matrix was renormalized after gene filtering. The data was log transformed after adding a pseudocount, and scaled to unit variance and zero mean. The dimensionality of the data was reduced by PCA (30 components) first and then with UMAP, followed by Louvain clustering performed on the 30 principal components with default parameters. For Louvain clustering, we first fitted the top 30 PCs to compute a neighborhood graph of observations with local neighborhood number of 50 by scanpy.api.pp.neighbors function in scanpy/v1.0. We then cluster the cells into sub-groups using the Louvain algorithm implemented as scanpy.api.tl.louvain function. For UMAP visualization, we directly fit the PCA matrix into scanpy.api.tl.umap function with min_distance of 0.1. For subcluster identification, we selected cells in each major cell type and applied PCA, UMAP, Louvain clustering similarly to the major cluster analysis. Subclusters with a detected doublet ratio (by Scrublet) over 15% were annotated as doublet-derived subclusters.

For data visualization, cells labeled as doublets (by Scrublet) or from doublet-derived subclusters were filtered out. For each cell, we only retain protein-coding genes, lincRNA genes and pseudogenes. Genes expressed in less than 10 cells and cells expressing less than 100 genes were further filtered out. The downstream dimension reduction and clustering analysis were done by Monocle 3. The dimensionality of the data was reduced by PCA (50 components) first on the top 5,000 most highly dispersed genes and then with UMAP (max_components=2, n_neighbors=50, min_dist=0.1, metric=‘cosine’). Cell clusters were identified using the Louvain algorithm implemented in Monocle 3 (louvain_res=1e-04). Clusters were assigned to known cell types based on cell type specific markers. We found the above Scrublet and iterative clustering based approach is limited in marking cell doublets between abundant cell clusters and rare cell clusters (e.g. less than 1% of total cell population). To further remove these doublet cells, we took the cell clusters identified by Monocle 3 and first computed differentially expressed genes across cell clusters (within-organ) with the differentialGeneTest( ) function of Monocle 3. We then selected a gene set combining the top ten gene markers for each cell cluster (ordered by q-value and fold expression difference between first and second ranked cell cluster). Cells from each main cell cluster were selected for dimension reduction by PCA (10 components) first on the selected gene set of top cluster specific gene markers, and then by UMAP (max_components=2, n_neighbors=50, min_dist=0.1, metric=‘cosine’), followed by clustering identification using the density peak clustering algorithm implemented in Monocle 3 (rho thresh=5, delta_thresh=0.2 for most clustering analysis). Subclusters showing low expression of target cell cluster specific markers and enriched expression of non-target cell cluster specific markers were annotated as doublets derived subclusters and filtered out in visualization and downstream analysis. Differentially expressed genes across cell types (within-organ) were re-computed with the differentialGeneTest( ) function of Monocle 3 after removing all doublets or cells from doublets derived subclusters.

Clustering Analysis of Cells Across Organs

For clustering analysis of 77 main cell types across 15 organs, we sampled 5,000 cells from each cell type (or all cells for cell types with fewer than 5,000 cells in a given organ). The dimensionality of the data was reduced first by PCA (50 components) on the gene set combining top cell type specific gene markers identified above (Table S5, qval=0) and then with UMAP (max_components=2, n_neighbors=50, min_dist=0.1, metric=‘cosine’). Differentially expressed genes across cell types were identified with the differentialGeneTest( ) function of Monocle 3. For annotating cell type specific gene features, we intersected the cell type specific genes identified above with the predicted secreted and membrane protein coding gene sets from the human protein atlas, as well as the TF set annotated in the “motifAnnotations_hgnc” data from package RcisTarget/v1.2.1.

For clustering analysis of blood cell across 15 organs, we extracted all blood cells including Myeloid cells, Lymphoid cells, Thymocytes, Megakaryocytes, Microglia, Antigen presenting cells, Erythroblasts, and Hematopoietic stem cells. The dimensionality of the data was reduced first by PCA (40 components) on the expression of a gene set combining the top 3,000 blood cell type specific gene markers (, only genes specifically expressed in at least one blood cell type were selected (q-value<0.05, fold expression difference between first and second ranked cell cluster>2) and ordered by median qval across organs) and then with UMAP (max_components=2, n_neighbors=50, min_dist=0.1, metric=‘cosine’). Cell clusters were identified using the Louvain algorithm implemented in Monocle 3 (louvain_res=1e-04). Clusters were assigned to known cell types based on cell type specific markers.

We then applied similar analysis strategy as above for clustering analysis of endothelial or epithelial cells across organs. For endothelial cells, we first extracted cells from Vascular endothelial cells, Lymphatic endothelial cells and Endocardial cells across organs. The dimensionality of the data was reduced first by PCA (30 components) on the gene set combining top 1,000 endothelial cell type specific gene markers identified above (only genes specifically expressed in at least one endothelial cell type were selected (q-value<0.05, fold expression difference between first and second ranked cell cluster>2) and ordered by median qval across organs) and then with UMAP with the same parameters of blood cells. Cell clusters were identified using the Louvain algorithm implemented in Monocle 3 (louvain_res=1e-04), and then annotated based on the tissue origin of endothelial cells. For epithelial cells, we first extracted cells from the epithelial cell cluster in FIG. S3B, followed by dimension reduction first by PCA (50 components) first on the top 5,000 most highly dispersed genes and then with UMAP (max_components=2, n_neighbors=50, min_dist=0.1, metric=‘cosine’).

TF-Gene Linkage Analysis

We hypothesized the gene regulatory process could be entangled from large-scale single cell gene expression analysis. Towards this goal, we applied a similar single cell regulatory inference method as previous study, to predict TF-gene interactions by coupling their covariance across millions of cells with regulatory sequence analysis for validation. The workflow consists of three steps: As the sparsity of our single cell profiles makes it challenging, we first aggregated the gene count from subsets of cells (˜100 cells) with highly similar transcriptome, by grouping cells (within organ) into subclusters by the iterative clustering strategy described above, followed by k-means clustering on UMAP coordinates for cells from each sub-cluster. The k is selected based on the number of cells in each subcluster so that the average cell number per subcluster is 100.

We sought to identify links between TFs and their regulated genes based on expression covariance across aggregated “pseudo-cells” within each organ. Cells with more than 10,000 UMIs detected, and genes (including TFs) detected in more than 10% of all cells were selected. The full gene expression per cell were normalized by cell-specific library size factors computed on the full gene expression matrix by estimateSizeFactors in Monocle 3, log transformed, centered, then scaled by scale function in R. For each gene detected, a LASSO regression model was constructed with package glmnet/v.2.0 to predict the normalized expression levels of each gene, based on the normalized expression of TFs annotated in the “motifAnnotations_hgnc” data from package RcisTarget/v1.2.1, by fitting the following model:

Gᵢ=β₀+βₜTᵢ

where Gᵢ is the adjusted gene expression value for gene i. It is calculated by the gene count for each pseudo-cell, normalized by cell specific size factor (SGᵢ) estimate by estimateSizeFactors in Monocle 3 on the full expression matrix of each pseudo-cell, and log transformed:

$${G}_{i} = ln ( \frac{{g}_{i}}{S {G}_{i}} + 0.1 )$$

To simplify downstream comparison between genes, we standardize the response Gᵢ prior to fitting the model for each gene i with the scale( ) function in R.

Similar with Gᵢ, Tᵢ is the adjusted TF expression value for each pseudo-cell. It is calculated by the full TF expression count, normalized by cell specific size factor (SGᵢ) estimate by estimateSizeFactors in Monocle 3 on the full expression matrix of each pseudo-cells, and log transformed:

$${T}_{i} = ln ( \frac{{t}_{i}}{S {G}_{i}} + 0.1 )$$

Prior to fitting, Tᵢ is standardized with the scale( ) function in R.

Although negative correlations between a TF's expression and a gene's new synthesis rate could reflect the activity of a transcriptional repressor, we felt that the more likely explanation for negative links reported by glmnet was mutually exclusive patterns of cell-state specific expression and TF activity. Thus during prediction, we excluded TFs with negatively correlated expression with a potential target gene's synthesis rate, and also low regression coefficient (<0.03) links.

Our approach aims to identify TFs that may regulate each gene, by finding the subset that can be used to predict its expression in a regression model. However, a TF with expression correlated with a gene's expression does not definitively mean that it is directly regulating that gene. To identify putatively direct targets within this set, we first intersected the links with TFs profiled in ENCODE ChIP-seq experiments. Only gene sets with significant enrichment of the correct TF ChIP-seq binding sites were retained (two-sided Fisher's Exact test, FDR of 5%), and further pruned to remove indirect target genes without TF binding data support. To expand the set of validated TF-gene links, we further applied package SCENIC, a pipeline to construct gene regulatory networks based on the enrichment of target TF motifs in the 10 kb window around genes' promoters. Each co-expression module identified by LASSO regression was analyzed using cis-regulatory motif analysis using RcisTarget/v1.2.1. Only modules with significant motif enrichment of the correct TF regulator were retained, and pruned to remove indirect target genes without motif support. We filtered the TF-gene links by three correlation coefficient thresholds (0.3, 0.4 and 0.5), and combined all links validated by RcisTarget³⁶ and ChIP-seq binding data.

We applied the above strategy to the aggregated pseudo-cells in each organ and identified 1,220 (Thymus) to 10,059 (Liver) TF-gene links across organs, which combined to a total of 56,272 TF-gene links between 706 TFs and 12,868 genes, validated by both expression covariance and TF binding or motif data. As a control analysis, we permuted the cell IDs of the TF expression matrix and no links were identified after permutation. Some of the identified TF and gene regulatory relationships are readily validated in a manually curated database of TF networks (TRRUST) or Enrichr submission TF-gene co-occurrence networks), such as E2F1 (top enriched TRRUST TF of 330 linked genes=E2F1, adjusted p-value=2.2e-14), HNF4A (top enriched TRRUST TF of 745 linked genes=HNF4A, adjusted p-value=0.000003), and FLI1 (top enriched co-occurrence TF of 1219 linked genes=FLI1, adjusted p-value=5.6e-122). 85% (48,050 out of 56,272) TF-gene links were organ specific. For example, ATPase Phospholipid Transporting 8B1 (ATP8B1) was linked to HNF4A only in intestine, consistent with the fact that it showed the highest correlations with HNF4A in intestine (Spearman correlation coefficient=0.36) compared with other organs (Mean of spearman correlation coefficient=0.008). 745 TF-gene links were found in multiple organs (>5). As expected, their linked genes were enriched in immune cell differentiation pathways (Hematopoietic Stem Cell Differentiation: adjusted p-value of 2.5e-6; Development of pulmonary dendritic cells and macrophage subsets: adjusted p-value of 0.0001) as well as basic biological processes such as stress response and cell cycle (DNA IR-damage and cellular response via ATR: adjusted p-value of 0.006, Oxidative Stress: adjusted p-value of 0.02, G1 to S cell cycle control: adjusted p-value of 0.05). 10.5% (5935 out of 56,272) TF-gene links were between two TFs, of which 362 TF pairs showed bi-directional regulatory relations potentially representing self-activation circuits. For example, we identified the positive feedback loops of key regulators driving skeletal muscle differentiation including MYOD1, MYOG, TEAD4 and MYF6. The cell type specific genes, TFs and their regulatory interactions can be visualized and explored in our website.

Human-Mouse Integration Analysis

We first applied a slightly modified strategy to identifying correlated cell types between human fetal cell atlas and mouse organogenesis cell atlas (MOCA). We first aggregate the cell type specific UMI counts, normalized by the total count, multiplied by 100,000, and log transformed after adding a pseudo-count. We then applied non-negative least squares (NNLS) regression to predict the gene expression of target cell type (Tₐ) in dataset A with the gene expression of all cell types (M_{b}) in dataset B:

Tₐ=β₀ₐ+β₁ₐM_{b}

where Tₐ and M_{b} represent filtered gene expression for target cell type from data set A and all cell types from data set B, respectively. To improve accuracy and specificity, we selected cell type-specific genes for each target cell type by: 1) ranking genes based on the expression fold-change between the target cell type vs. the median expression across all cell types, and then selecting the top 200 genes. 2) ranking genes based on the expression fold-change between the target cell type vs. the cell type with maximum expression among all other cell types, and then selecting the top 200 genes. 3) Merge the gene lists from step (1) and (2). β₁ₐ is the correlation coefficient computed by NNLS regression.

Similarly, we then switch the order of datasets A and B, and predict the gene expression of target cell type (T_{b}) in dataset B with the gene expression of all cell types (Mₐ) in dataset A:

T_{b}=β_{0b}+β_{1b}Mₐ

Thus, each cell type a in dataset A and each cell type b in dataset B are linked by two correlation coefficients from the above analysis: β_{ab} for predicting cell type a using b, and β_{ba} for predicting cell type b using a. We combine the two values by:

β=β_{ab}+β_{ba}

and find β reflects the matching of cell types between two data sets with high specificity. For each cell type in dataset A, all cell types in dataset B are ranked by β and the top cell type (with β>0.06) is identified as the matched cell type. We compared all human cell types from this study to 10 main cell trajectories and 56 sub-trajectories from the mouse embryonic cell atlas (MOCA).

We then integrated the human fetal cell atlas and the mouse organogenesis cell atlas (MOCA) using the Seurat v3 integration method (FindAnchors and IntegrateData) with a chosen dimensionality of 30 on the top 3,000 highly variable genes with shared gene names in both human and mouse. We first integrated 65,000 human fetal cells (up to 1,000 cells randomly sampled from each of 77 cell types) and 100,000 mouse embryonic cells randomly sampled from MOCA with default parameters. We then applied the same integrative analysis strategy to extracted human and mouse cells from haematopoietic, endothelial and epithelial trajectories.

### Example 3

Method for Single Cell Profiling of Chromatin Accessibility Based on Three-Level Combinatorial Indexing (Sci-ATAC-Seq

Materials

Reagents and Consumables

0.5 M EDTA (Thermo Fisher Scientific, AM9260G); 100 bp ladder (New England Biolabs (NEB), N323IL); 1000× Sybr (Invitrogen (Gibco/BRL Life Tech), S7563); 10 mM ATP (New England Biolabs (NEB), PO756S); 10×HBSS (Gibco/BRL Life Tech, 14065-056); 10×PNK Buffer (New England Biolabs (NEB), M0201L); 1M MgCl2 (Thermo Fisher Scientific, AM9530G); 1×DPBS (Thermo Fisher Scientific, 14190-144); 5% Digitonin (Thermo Fisher Scientific, BN2006); 5M NaCl (Thermo Fisher Scientific, AM9759); 6% TBE PAGE (Invitrogen (Gibco/BRL Life Tech), EC6265BOX); 6× Orange dye (New England Biolabs (NEB), B7022S); AMPure Beads (Beckman Coulter, A63882); BSA, Molecular Biology Grade (New England Biolabs (NEB), B9000S); DNA LoBind Tube 1.5 ml, PCR clean (Eppendorf North America, 22431021); DL-Dithiothreitol, 1 M 10×0.5 ML (Sigma Aldrich, 64563-10×0.5 ML), EB Buffer (Qiagen, 19086); Falcon Tubes, 15 ml (VWR Scientific, 21008-936); Falcon Tubes, 50 ml (VWR Scientific, 21008-940); Falcon® 5 mL Round Bottom w/Cell Strainer (Fisher Scientific, 352235); Green pack LTS 200 ul filter tips (GP-L200F) (Rainin Instrument, 17002428); Green pack LTS 20 ul filter tips (GP-L20F) (Rainin Instrument, 17002429); Glycerol (Sigma Aldrich, G5516-500ML); Glycine (Sigma Aldrich, 50046-250G); IGEPAL CA-630 (Sigma Aldrich, I8896-50ML); Liquidator tips—10 ul (Rainin Instrument, 17011117); Liquidator tips—200 ul (Rainin Instrument, 17010646); LoBind clear, 96-well PCR Plate (Eppendorf North America, 30129512); Low-Profile 0.2 ml 8-tube white tube w/o cap (Bio-rad Laboratories, TLS0851); Magnesium acetate tetrahydrate (Sigma Aldrich, M5661-50G); Microseal ‘B’ Adhesive seal (Bio-Rad Laboratories, MSB1001); Nalgene MF 75 Sterilization Filter Unit, 0.2 um-250 ml (VWR, 28199-112); Nalgene MF 75 Sterilization Filter Unit, 0.2 um-500 ml (VWR, 28198-505); NEBNext Hi-fidelity master mix (2×) (New England Biolabs (NEB), M0541L); NextSeq 500 High Output kit (150 cycle) (Illumina Inc., FC-404-2002); Non-woven gauze (Dukal, 6114); Nuclease-free water (Thermo Fisher Scientific, AM9937); Optical flat 8-cap strips (Bio-Rad Laboratories, TCS-0803); Protease Inhibitors (Sigma Aldrich, P8340-1 ml); RT-L250WS wide-orifice LTS 250 ul (Rainin Instrument, 30389249); Reagent reservoirs (Fisher Scientific, 07-200-127); Spermidine (Sigma Aldrich, S2626-1G); Sybr gold (Invitrogen (Gibco/BRL Life Tech), S-11494); Steriflip, Disposable Vacuum Filter Unit, 0.22 um pore (Fisher Scientific, SCGP00525); T4 PNK (New England Biolabs (NEB), M0201L); T7 Ligase (New England Biolabs (NEB), M0318L); T7 Ligase Buffer (New England Biolabs (NEB), M0318L); Tapestation (D5000 reagent) (Agilent Technologies, 5067-5589); Tapestation (screentape) (Agilent Technologies, 5067-5588); TD Buffer (2×) (Illumina Inc., FC-121-1031); TDE1 (Tn5) (Illumina Inc., FC-121-1031); Tris-HCl pH 7.5 (1M) (Thermo Fisher Scientific, 15567027); Tween-20 (Thermo Fisher Scientific, BP337-500); UltraPure Distilled Water (DNAse, RNAse, Free) (Thermo Fisher Scientific, 10977023); DNA Clean and Concentrate (DCC-5) (Zymo Research, D4014).

Instruments:

Agilent 4200 TapeStation System; Bright-Line™ Hemacytometer (Sigma); Centrifuge (cooled to 4° C.) (Eppendorf, 5810R); DynaMag™-96 Side Skirted Magnet (Thermo Fisher Scientific, 12027); Eppendorf Mastercycler (thermal cycler); FACSAria III cell sorter (BD); Freezer (−20° C., −80° C.) and refrigerator (4° C.); Gel box; Liquid nitrogen tank for sample storage; Microscope; Multi-channel pipettes (10 ul, 200 ul) (Rainin Instrument); NextSeq 500 platform (Illumina); Rainin Liquidator 96 Manual Pipetting System

Reagent Preparation:

The ATAC-RSB recipe was used. In a 50 ml falcon tube, combine 500 ul 1M Tris-HCl pH 7.4 (10 mM Tris-HCl final), 100 ul 5M NaCl (10 mM NaCl final), 300 ul 0.5M MgCl2 (3 mM MgCl2 final) and 49.1 ml nuclease free water. Filter sterilize by using Millipore “Steritlip” Sterile Disposable Vacuum Filter Unit, PES membrane; Pore size: 0.22 um (SCGP00525). Store bufter at 4° C. for up to 6 months.

10% Tween −20 (store in 4° C. for up to 6 months); 10% IGEPAL CA-630 (store in 4° C. for up to 6 months); 1% Digitonin (dilute 5% digitonin to 1% with nuclease-free water, store in 4° C. for up to 6 months)

Freezing buffer (FB). In a 50 mil falcon tube, combine 50 mM Tris at pH 8.0, 25% glycerol, 5 mM Mg(OAc)2, 0.1 mM EDTA, and water. Filter sterilize by using Millipore “Steritlip” Sterile Disposable Vacuum Filter Unit, PES membrane; Pore size: 0.22 um (SCGP00525). Store buffer at 4° C. for up to 6 months. On the day of nuclei isolation, mix 975 ul of FB, 5 ul 5 mM DTT (Sigma-Aldrich cat. no. 646563-10×0.5 ml) and 20 ul 50× protease inhibitor cocktail (Sigma-Aldrich cat. No. P8340).

2.5 M glycine Make 2 5M glycine, combine 46.92 g of glycine in 250 ml of water then filter sterilize (Nalgene filtration system, 0.2 um cellulose nitrate membrane (VWR, 28199-112) Store reagent at room temperature for up to 6 months.

40 mM EDTA Make 40 mM EDTA from 0.5M EDTA stock (Invitrogen, AM9262) with water then filter sterilize (VWR, 28198-505). Store reagent at room temperature for up to 6 months.

Cell culture. GM12878 cells were cultured and maintained in RPMI 1640 medium (Thermo Fisher Scientific cat no. 11875-093) with 15% FBS (Thermo Fisher cat. no. SH30071.03) and 1% Pen-strep (Thermo Fisher cat. no 15140122). Count and split at 300,000 cells/ml three times a week. CH12-LX murine cell line were cultured in RPMI 1640 medium with 10% FBS, 1% Pen-strep (Penicillin and Streptomycin) and 1×10\hat{ }5M B-ME. They were counted and maintained at a density of 1×10\hat{ }5 cells/ml, splitting three times a week to maintain cell concentration. Both cell lines were incubated at 37° C. with 5% CO₂.

Nuclei isolation and fixation from cell lines. For suspension cells, obtain between ˜10-100 million cells and pellet cells by spinning at 500×g for 5 min at room temperature. Aspirate supernatant and resuspend pellet in 1 ml Omni-ATAC lysis buffer (10 mM NaCl, 3 mM MgCl2, 10 mM Tris-HCl pH 7.4, 0.1% NP40, 0.1% Tween 20 and 0.01% Digitonin) and incubate on ice for 3 min. Add 5 ml of 10 mM NaCl, 3 mM MgCl2, 10 mM Tris-HCl pH 7.4 with 0.1% Tween 20 and pellet nuclei for 5 min at 500×g at 4° C. Aspirate supernatant and resuspend nuclei in 5 ml 1×DPBS (Thermo Fisher cat. no. 14190144). To crosslink the nuclei, add 140 ul of 37% formaldehyde with methanol (VWR cat. no. MK501602) in one shot at a final concentration of 1%. Incubate fixation mixture at room temperature for 10 minutes inverting every 1-2 min. To quench the crosslinking reaction, add 250 ul of 2.5 M glycine and incubate at room temperature for 5 minutes and then on ice for 15 minutes to stop cross-linking completely. Take 20 ul of the quenched crosslinked mixture to 20 ul of trypan blue for counting. Spin crosslinked nuclei at 500×g for 5 minutes at 4° C. and aspirate the supernatant. Resuspend fixed nuclei in appropriate amount of freezing buffer (50 mM Tris at pH 8.0, 25% glycerol, 5 mM Mg(OAc)₂, 0.1 mM EDTA, 5 mM DTT (Sigma-Aldrich cat. no. 646563-10×0.5 ml), 1× protease inhibitor cocktail (Sigma-Aldrich cat. No. P8340) to obtain 2 million nuclei per 1 ml aliquot, snap freeze in liquid nitrogen and store in −80° C.

Tissue Procurement and Storage.

Isolate tissue of interest. Rinse in 1×HBSS pH 7.4 (with Ca, with Mg), 1×HBSS with calcium and magnesium, no phenol red, Gibco BRL (500 ml) 14065-056. Blot tissue dry on semi-damp gauze (wet gauze prevents tissue from sticking to the gauze) Non-woven gauze Dukal #6114. Place dried tissue on heavy duty foil (NC19180132, Fisher Scientific) or in cryotube. Note: cryotubes can create “frost” of water crystals inside the tube due to trapped air/moisture during the snap-freeze process Snap freeze tissue using liquid nitrogen. Store tissue in repository at −80° C.

Pulverization and storage. On day of pulverization, pre-cool pre-labeled tubes and hammer on dry ice with a cloth towel between the dry ice and metal. Create a “padding” by taking an 18″×18″ heavy duty foil, fold in half twice creating a rectangle. Fold twice more to create a square. Place frozen tissue inside the foil “padding” then place tissue in foil padding inside a pre-chilled 4 mm plastic bag to prevent tissue from falling out onto the dry ice in case the foil rupture. Chill this tissue packet between 2 slabs of dry ice. Using the pre-chilled hammer, manually pulverize the tissue inside the packet; 3 to 5 impacts avoiding a grinding motion before taking a break to avoid heating the sample. Chill the hammer and repeat pulverization as needed until tissue is uniform. Aliquot pulverized tissue into pre-labeled and pre-chilled 1.5 ml LoBind and nuclease-free snap cap 1.5 ml tubes (Eppendorf cat. no. 022431021). Aliquots of powdered tissues can be stored at −80° C. until further processing.

Nuclei isolation and fixation of frozen tissues. Before starting, prepare Omni lysis buffer (RSB+0.1% Tween+0.1% NP-40 and 0.01% Digitonin) and RSB with 0.1% Tween-20. On day of nuclei isolation, add lysis buffer directly to tube or pour out the frozen aliquot into a 60 mm dish with cell lysis buffer and further mince with a blade. As long as the aliquot has not thawed at some point in storage, the powdered tissue aliquot should easily slide out of the storage tube without sample loss. An estimated ˜20,000 cells per mg of original tissue weight can be obtained, and performance may vary from tissue to tissue. Resuspend pulverized tissue in 1 ml Omni lysis (RSB+0.1% Tween+0.1% NP-40 and 0.01% Digitonin) then transfer to a 15 ml falcon tube. Incubate nuclei for 3 minutes on ice then add 5 ml RSB+0.1% Tween-20. Centrifuge the nuclei at 500×g for 5 minutes at 4° C. Aspirate supernatant and resuspend in 5 ml 1×DPBS. Pass through nuclei in 1×DPBS into a 100 um cell strainer (VWR cat. no 10199-658) to remove tissue clumps.

In the fume hood, crosslink the nuclei by adding 140 uL of 37% formaldehyde (VWR, MK501602) with methanol in one shot for 1% final concentration and mixing quickly by inverting the tube several times. Incubate at room temperature for exactly 10 minutes with gently inversion of the tube every 1-2 min Add 250 uL of 2.5M glycine (freshly-made, filter-sterilized) to quench the cross-linking reaction, mix well by inverting the tube several times. Incubate for 5 minutes at room temperature, then on ice for IS minutes to stop the cross-linking completely. Count nuclei using hemocytometer to know final volume of freezing buffer to add, the goal is to freeze ˜1-2 million nuclei/tube. Centrifuge the cross-linked nuclei at 500×g for 5 minutes at 4° C., aspirate the supernatant and resuspend pellet in 1-10 ml of freezing buffer supplemented with 1× protease inhibitors and 5 mM DTT. Snap-freeze nuclei in liquid nitrogen and store nuclei at −80° C.

sci-ATAC-seq3 sample processing (library construction and qc) Thawing, permeabilization, counting and tagmentation. Before starting, prepare Omni lysis buffer (RSB+0.1% Tween+0.1% NP-40 and 0.01% Digitonin) and RSB with 0.1% Tween-20. Take frozen fixed nuclei out of the −80′C and place on a bed of dry ice. Thaw nuclei in 37° C. water bath until thawed (˜30 sec-1 min) and transfer nuclei into a 15 ml falcon tube. Pellet nuclei at 500×g for 5 minutes at 4° C. Aspirate supernatant without disturbing the pellet and resuspend pellet in 200 ul of Omni lysis buffer then incubate on ice for 3 minutes. Wash out lysis buffer with 1 ml ATAC-RSB with 0.1% Tween-20 and gently invert tube 3 times to mix. Count nuclei by taking 20 ul of nuclei and 20 ul of Trypan blue. While counting, keep nuclei on ice whenever possible from here on. For 3 level indexing experiments at 384\hat{ }3, nuclei input number is 4.8 million @ 50,000 nuclei per well per tissue or sample spread across 96 reactions. Per batch, there are 23 samples/tissues plus a mixture of mouse and human nuclei as a 24^{th} sample and control. Make a master mix for tagmentation reaction (Table 1):

TABLE 1

| Nuclei count | 1X | X 110 |
|---|---|---|
| Nuclei amount used | | |
| TD buffer 2X (110 rxn) | 25 | 2750 |
| 1X DPBS | 8.25 | 907.5 |
| 1% Digitonin | 0.5 | 55 |
| 10% Tween-20 | 0.5 | 55 |
| Water | 13.25 | 1457.5 |
| NexteraV2 enzyme | 2.5 | |
| Total | 50 | 5225 |

For each sample, take 225,000 nuclei (based on count), spin at 500×g for 5 min at 4° C., aspirate supernatant and resuspend pellet in 213 ul of pre-made tagmentation reaction master mix. Aliquot 47.5 ul of nuclei in tagmentation mix using wide bore tip (Rainin Instrument Co cat. no. 30389249) across 4 wells of LoBind 96 well plate (Eppendorf cat. No. 30129512). Add 2.5 ul of Nextera v2 enzyme (Illumina Inc cat. no. FC-121-1031) per well, seal plate with adhesive tape and spin at 500×g for 30 sec. Incubate plate at 55′C for 30 minutes to tagment DNA. Make stop reaction master mix by combining 25 ml of 40 mM EDTA and 3.9 ul of 6.4 M Spermindine (final 20 mM EDTA and 1 mM Spermidine). Tagmentation reactions were stopped by adding 50 ul of stop reaction mixture 40 mM EDTA with 1 mM Spermidine then incubated at 37° C. for 15 min

Pooling, PNK reaction and N5 ligation. Using wide-bore tips, tagmented nuclei were pooled (per sample) and pelleted at 500×g for 5 minutes at 4° C. and then washed with 500) ul of ATAC-RSB with 0.1° % Tween-20. Pellet nuclei in 500× g for 5 minutes at 4° C. aspirate supernatant and resuspend in 18 ul ATAC-RSB with 0.1% Tween-20 per sample. Create a PNK reaction master mix (Table 2):

TABLE 2

| | | 440x |
|---|---|---|
| 10x PNK buffer | 0.5 | 220 |
| rATP 10 mM | 0.5 | 220 |
| water | 1 | 440 |
| T4 PNK | 2 | 880 |

Add 72 ul of PNK master mix to each sample. Aliquot 5 ul of PNK reaction mix (across 16 wells across four 96 well plates). Seal with adhesive tape and spin at 500×g for 5 minutes at 4° C. PNK reaction were incubated at 37° C. for 310 minutes. Create a N5 ligation master mix enough for 440 reactions (Table 3):

TABLE 3

| | | 440x | |
|---|---|---|---|
| PNK reaction with nuclei | 5 | | |
| 2X T7 ligase buffer | 10 | 4400 | |
| 1000 uM_N5_splint | 0.18 | 80 | |
| water | 1.12 | 492.8 | |
| T7 DNA ligase | 2.5 | 1100 | |
| 50 uM_N5_oligo | 1.2 | | Add separately |

Using a multichannel, directly add 13.8 ul of ligation master mix to each PNK reaction. Using a multi-channel or a 96 head dispenser (Liquidator, cat. No. 17010335), add 1.2 ul of 50 uM N5_oligo (IDT) to each well across four 96 well plates. Seal with adhesive tape and spin at 500×g for 30 seconds then incubate at 25° C. for 1 hour After first round of ligation, add 20 ul of EDTA and Spermidine mix (20 mM EDTA and 1 mM Spermidine) to stop ligation reaction and incubate at 37° C. for 15 minutes. Using wide bore tips, pool each well in a trough and transfer into a 50 ml falcon tube. Pellet nuclei at 500×g for 5 minutes at 4° C., aspirate supernatant and resuspend nuclei in 1 ml of ATAC-RSB with 0.1% Tween-20 to wash any residual ligation reaction mix. Pellet nuclei at 500× g for 5 minutes at 4° C. and aspirate supernatant without disturbing the pellet.

N7 ligation. Create N7 ligation master mix enough for 440 reactions (1×T7 ligase buffer, 9 uM N7_splint (IDT), water and T7 DNA ligase) and resuspend the nuclei with the ligation master mix (Table 4).

TABLE 4

| | | 440x | |
|---|---|---|---|
| 2X T7 ligase buffer | 10 | 4400 | |
| 1000 uM_N7_splint | 0.18 | 80 | |
| water | 6.12 | 2692.8 | |
| T7 DNA ligase | 2.5 | 1100 | |
| 50 uM_N7_oligo | 1.2 | | Add separately |

Transfer nuclei suspended in master mix into a trough and using wide bore tips, aliquot 18.8 ul of ligation master mix into four 96 well LoBind plates then add 1.2 ul of 50 uM N7_oligo (IDT) to each well across four 96 well plates. Seal plates with adhesive tape and spin at 500×g for 30 seconds then incubate at 25° C. for 1 hour Stop ligation by adding 20 ul of EDTA and Spermidine mix (20 mM EDTA and 1 mM Spermidine) and incubate at 37° C. for 15 minutes.

Pooling, counting and dilution. Pool wells in a trough using wide bore tips and then transfer into a 50 ml falcon tube Pellet nuclei at 500×g for 5 minutes at 4° C., aspirate supernatant and resuspend nuclei in 2 ml of Qiagen EB buffer (Qiagen cat. no. 19086). Filter nuclei using FACs tube with 40 um filtered cap (Fisher Scientific cat. no #352235) Take 20 ul of resuspended and filtered nuclei and 20 ul of trypan blue to count nuclei. Dilute nuclei to 100-300 nuclei per ul and aliquot 10 ul per well into four 96 well LoBind plates.

Uncrosslinking. To reverse crosslink the nuclei, make a reverse crosslink master mix of EB buffer, Proteinase k (Qiagen, cat. no. 19133) and 1% SDS; 1 ul/0.5 ul/0.5 ul respectively per well) and add 2 ul to each well of nuclei Seal with adhesive tape, spin at 500×g for 30 seconds and incubate at 65° C. for 16 hours.

Test PCR and gel QC. Briefly spin down uncrosslinked plates before starting. Make a PCR master mix enough for 6 reactions (Table 5):

TABLE 5

| | | Master mix (6x) |
|---|---|---|
| Uncrosslinked nuclei | 12.0 | |
| P7_flipmod_10 uM_row_{—} | 1.25 | Add separately |
| P5_flipmod_10 uM_column_{—} | 1.25 | Add separately |
| NEBNext Hi-Fidelity 2x Master Mix | 25 | 150 |
| 100X BSA | 1.0 | 6 |
| 100X SYBR Green | 0.25 | 1.5 |
| Water | 9.25 | 55.5 |

Aliquot 35.5 ul of PCR master mix into an 8-strip tube white without cap (Bio-Rad Laboratories, TLS0851). Add 1.25 ul of 10 uM P7 and P5 primers. Add 12 ul of uncrosslinked nuclei to the PCR and primer mix. Cap reaction tubes with optical flat 8-cap strips (Bio-Rad Laboratories, TCS-0803). Place in qPCR machine and monitor amplification to determine optimal cycle number: 72° C. 5 min, 98° C. 30 sec, 30 Cycles of 98° C. 10 sec, 63° C. 30 sec, 72° C. 1 min, then hold at 10° C. Based on the test wells, choose cycle number such that test wells were all clearly amplifying but before the fluorescence intensity in any of the wells has saturated. Take 1 ul of PCR product for QC: Samples=1 ul+9 ul nuclease free water+2 ul 6× orange dye; 100 bp ladder (1:10)=1 ul+9 ul nuclease free water+2 ul 6× orange dye. Run 6% TBE polyacrylamide gel, 180 volts for 35 mins. Stain with 5 ul SYBR Gold and 50 ml 0.5×TBE buffer, room temperature for 5 min.

PCR plate set-up. Briefly spin down plate. Set aside on ice until test PCR result becomes available. Make a PCR master mix (Table 6):

TABLE 6

| | | Master mix (110x) |
|---|---|---|
| Uncrosslinked nuclei | 12.0 | |
| P7_flipmod_10 uM_row_{—} | 1.25 | Add separately |
| P5_flipmod_10 uM_column_{—} | 1.25 | Add separately |
| NEBNext Hi-Fidelity 2x Master Mix | 25 | 2750 |
| 100X BSA | 1.0 | 110 |
| Water | 9.5 | 1045 |

Note row and column primer combination used during amplification. Seal with an adhesive tape then spin at 500×g for 30 sec. Run PCR plate with the optimal cycle number from test PCR result: 72° C. 5 min, 98° C. 30 sec, 10-20 Cycles: 98° C. 10 sec, 63° C. 30 sec, 72° C. 1 min, then hold at 10° C.

PCR Amplification Clean-up and QC. Clean PCR products with Zymo Clean&Concentrator-5. Combine 25 ul of each PCR reaction (2.4 ml) to a trough, Add 2 volumes binding buffer (4.8 ml), Split across 4 C&C columns (600 ul spun 3 times in each column), Add 200 ul Zymo wash buffer and spin (2 washes total), Use an extra spin to dry columns for 1 min after last wash, Elute in 25 ul Qiagen elution buffer (let buffer stand on column 1 min, then spin 1 min at max speed), Combine all 4 eluates and clean a second time in 1× AMPure beads (100 ul), Place on MPC (magnetic particle collector) until supernatant is clear, aspirate supernatant. Wash beads with 200 ul 80% ethanol twice, Dry beads for 30 sec-1 min until beads are dull in color without over drying the beads, Elute beads in 25 ul Qiagen EB buffer, place in MPC and transfer supernatant to a clean tube For library QC using Tapestation, follow manufacturer's specification using D5000 ScreenTape Assay. For fragment analysis, create a region table from 200-1000 bp in which a region molarity is calculated. Use that nM (nmol/l) concentration to dilute library to 2 nM with buffer EB and 0.1% Tween-20 If pooling multiple libraries, normalize each library to 2 nM and create an equimolar pool for sequencing.

Next Sequencing (150 cycle kit). Library denaturation: Dilute 2N NaOH to 0.2N NaOH (10 ul 1N to 90 ul nuclease-free water), In a new 1.5 Lo-Bind tube, transfer 10 ul 0.1N NaOH and add 10 ul 2 nM pooled libraries, Incubate at room temperature for 5 minutes, Add 980 ul HT1 to dilute denature libraries to 20 μM, Dilute denatured library to 1.8 μM loading concentration (135 ul 20 μM+1365 ul HT1), Dilute custom primers to 0.6 uM, NextSeq Sequencing recipe name: 3LV2_sciATAC_high.

R1-50 bases for gDNA, R2-50 bases for gDNA.

Index 1—20 bases (10 bases for N7 oligo, 15 dark cycle, 10 bases PCR barcode), Index 2—20 bases (10 bases for N5 oligo, 15 dark cycle, 10 bases PCR barcode).

Sequencing primers:
3L_NexteraV2_R1_seq
(SEQ ID NO: 5)
TCGTCGGCAGCGTCAGATGTGTATAAGAGACAG;
L_NexteraV2_R2_seq
(SEQ ID NO: 6)
GTCTCGTGGGCTCGGAGATGTGTATAAGAGACAG;
3LV2_IDX1
(SEQ ID NO: 7)
CTCCGAGCCCACGAGACGACAAGTC;
3LV2_IDX2
(SEQ ID NO: 8)
ACACATCTGACGCTGCCGACGACTGATTAC.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for identifying a subpopulation of cells comprising a biological feature, the method comprising:
(a) providing a single-cell sequencing library,
wherein the sequencing library comprises a plurality of modified target nucleic acids,
wherein the modified target nucleic acids comprise at least one index sequence;
(b) interrogating the sequencing library by targeted sequencing to identify the index sequences that are present on the same modified target nucleic acid as a biological feature,
wherein the index sequences associated with the biological feature are marker index sequences;
(c) altering the sequencing library to obtain a sub-library,
wherein the sub-library comprises increased representation of the modified target nucleic acids comprising the marker index sequences in comparison to other modified target nucleic acids present in the sequencing library that do not comprise a marker index sequence;
(d) determining the nucleotide sequence of the modified target nucleic acids comprising a marker index sequence.

2. The method of claim 1, wherein the single-cell sequencing library comprises nucleic acids from multiple samples.

3. The method of claim 2, wherein the multiple samples comprise (i) samples of the same tissue obtained from different organisms, (ii) samples of different tissues from one organism, or (iii) samples of different tissues from different organisms.

4. The method of claim 1, wherein more than one marker index sequence is identified in step (b).

5. The method of claim 1, wherein the single-cell combinatorial sequencing library comprises target nucleic acids representative of the whole genome of the cells or nuclei or a subset of the genome.

6. The method of claim 5, wherein the subset of the genome comprises target nucleic acids representative of transcriptome, accessible chromatin, DNA, conformational state, or proteins of the cells or nuclei.

7. The method of any one of claims 1-6, wherein the altering comprises enrichment of the modified target nucleic acids comprising the marker index sequences.

8. The method of claim 7, wherein the enriching comprises a hybridization-based method.

9. The method of claim 8, wherein the hybridization-based method comprises hybrid capture, amplification, or CRISPR (d) Cas9.

10. The method of claim 9, wherein the altering comprises depletion of the modified target nucleic acids that do not comprise the marker index sequences.

11. The method of claim 10, wherein the depletion comprises a hybridization-based method.

12. The method of claim 11, wherein the hybridization-based method comprises hybrid capture, amplification, or CRISPR (d) Cas9.

13. The method of claim 1, wherein the biological feature comprises a nucleotide sequence indicative of species type.

14. The method of claim 13, wherein the species type comprises the species of the cell.

15. The method of claim 14, wherein the biological feature comprises nucleotides of a 16s subunit, a 18s subunit, or an ITS non-transcriptional region.

16. The method of claim 1, wherein the biological feature comprises a nucleotide sequence indicative of cell class.

17. The method of claim 16, wherein the cell class comprises expression pattern, epigenetic pattern, immune gene recombination, or a combination thereof.

18. The method of claim 17, wherein the epigenetic pattern comprises methylation mark, methylation pattern, accessible DNA, or a combination thereof.

19. The method of claim 1, wherein the biological feature comprises a nucleotide sequence indicative of disease status or risk.

20. The method of claim 19, wherein disease status or risk comprises a variant DNA sequence, a variant expression pattern, or a variant epigenetic pattern that correlates with a disease.

21. The method of claim 20, wherein the variant DNA sequence comprises at least one single nucleotide polymorphism.

22. The method of claim 21, wherein the variant expression pattern comprises expression of a biomarker.

23. The method of claim 22, wherein the variant epigenetic pattern comprises a methylation mark, methylation pattern.

24. The method of claim 1, wherein the modified target nucleic acids comprise a contiguous index of at least 2 compartment-specific index sequences, wherein there are no greater than 6 nucleotides between the 2 index sequences.

25. The method of claim 24, wherein the contiguous index is present at each end of the modified target nucleic acids.

26. The method of claim 24 or 25, wherein the length of the contiguous index is at least 55 nucleotides.

27. The method of any one of claims 24-26, wherein one copy of the contiguous index is present on the modified target nucleic acids.

28. The method of any one of claims 24-26, wherein two copies of the contiguous index are present on the modified target nucleic acids.

29. The method of claim 1, wherein the plurality of modified target nucleic acids of the sequencing library is representative of at least 100,000 different cells or nuclei.

30. The method of claim 1, wherein the providing the single-cell combinatorial sequencing library comprises:
processing a sample to produce a library, wherein the sample is a metagenomics sample obtained from an organism.

31. The method of claim 30, wherein the organism is a mammal.

32. The method of claim 30 or 31, wherein the metagenomics sample comprises a tissue suspected of comprising a commensal or pathogenic microbe.

33. The method of claim 32, wherein the microbe is prokaryotic or eukaryotic.

34. The method of any one of claims 30, 31, or 33, wherein the metagenomics sample comprises a microbiome sample.

35. The method of claim 1, wherein the providing the single-cell combinatorial sequencing library comprises:
processing a sample to produce a library, wherein the sample is from an organism.

36. The method of claim 35, wherein the organism is a mammal.

37. The method of claim 35, wherein the primary source of nucleic acids from the sample comprise RNA.

38. The method of claim 37, wherein the RNA comprises mRNA.

39. The method of claim 35, wherein the primary source of nucleic acids from the sample comprise DNA.

40. The method of claim 39, wherein the DNA comprises whole cell genomic DNA.

41. The method of claim 40, wherein the whole cell genomic DNA comprises nucleosomes.

42. The method of claim 35, wherein the primary source of nucleic acids from the sample comprise cell free DNA.

43. The method of claim 35, wherein the sample comprises cancer cells.

44. The method of claim 1, wherein the providing the single-cell combinatorial sequencing library comprises a producing the library with a single-cell combinatorial indexing method selected from single-nuclei transcriptome sequencing, single-cell transcriptome sequencing, single-cell transcriptome and transposon-accessible chromatin sequencing, whole genome sequencing of single nuclei, single nuclei sequencing of transposon accessible chromatin, single-cell epitope sequencing, sci-HiC, and sci-MET.

45. The method of claim 44, wherein the providing comprises providing two different single-cell combinatorial sequencing libraries from each cell or nucleus.

46. The method of claim 45, wherein the two different single-cell combinatorial sequencing libraries are selected from a single-cell combinatorial indexing method selected from single-nuclei transcriptome sequencing, single-cell transcriptome sequencing, single-cell transcriptome and transposon-accessible chromatin sequencing, whole genome sequencing of single nuclei, single nuclei sequencing of transposon accessible chromatin, sci-HiC, and sci-MET.

47. The method of claim 1, further comprising performing a sequencing procedure to determine the nucleotide sequences for the nucleic acids.

48. A method for preparing a sequencing library comprising nucleic acids from a plurality of single nuclei or cells, the method comprising:
(a) providing a plurality of nuclei or cells, wherein the nuclei or cells comprise nucleosomes;
(b) contacting the plurality of nuclei or cells with a transposome complex comprising a transposase and a universal sequence, wherein the contacting further comprises conditions suitable for incorporation of the universal sequence into DNA nucleic acids resulting in double stranded DNA nucleic acids comprising the universal sequence;
(d) distributing the plurality of nuclei or cells into a first plurality of compartments,
wherein each compartment comprises a subset of nuclei or cells;
(e) processing DNA molecules in each subset of nuclei or cells to generate indexed nuclei or cells,
wherein the processing comprises adding to DNA nucleic acids present in each subset of nuclei or cells a first compartment specific index sequence to result in indexed nucleic acids present in indexed nuclei or cells,
wherein the processing comprises ligation, primer extension, hybridization, amplification, or a combination thereof, and
(g) combining the indexed nuclei or cells to generate pooled indexed nuclei or cells.

49. The method of claim 48, wherein the providing comprises providing the plurality of nuclei or cells in a plurality of compartments, wherein each compartment comprises a subset of nuclei or cells, wherein the contacting comprises contacting each compartment with the transposome complex, and wherein the method further comprises combining the nuclei or cells after the contacting to generate pooled nuclei or cells.

50. The method of claim 48, wherein the providing comprises subjecting the nuclei to a chemical treatment to generate nucleosome-depleted nuclei while maintaining integrity of the isolated nuclei.

51. The method of claim 48, further comprising:
distributing the pooled indexed nuclei or cells comprising the indexed nuclei or cells into a second plurality of compartments,
wherein each compartment comprises a subset of nuclei or cells;
processing DNA molecules in each subset of nuclei or cells to generate dual-indexed nuclei or cells,
wherein the processing comprises adding to DNA nucleic acids present in each subset of nuclei or cells a second compartment specific index sequence to result in dual-indexed nucleic acids present in indexed nuclei or cells,
wherein the processing comprises ligation, primer extension, hybridization, amplification, or a combination thereof,
combining the dual-indexed nuclei or cells to generate pooled dual-indexed nuclei or cells;

52. The method of claim 51, further comprising:
distributing the pooled nuclei or cells comprising the dual-indexed nuclei or cells into a third plurality of compartments,
wherein each compartment comprises a subset of nuclei or cells;
processing DNA molecules in each subset of nuclei or cells to generate triple-indexed nuclei or cells,
wherein the processing comprises adding to DNA nucleic acids present in each subset of nuclei or cells a third compartment specific index sequence to result in triple-indexed nucleic acids present in indexed nuclei or cells,
wherein the processing comprises ligation, primer extension, hybridization, amplification, or a combination thereof,
combining the triple-indexed nuclei or cells to generate pooled triple-indexed nuclei or cells.

53. The method of any one of claims 48, 51, or 52, wherein the distributing step comprises dilution.

54. The method of any one of claims 48, 51, or 52, wherein the compartment comprises a well, microfluidic compartment, or a droplet.

55. The method of claim 48, wherein compartments of the first plurality of compartments comprise from 50 to 100,000,000 nuclei or cells.

56. The method of claim 51, wherein compartments of the second plurality of compartments comprise from 50 to 100,000,000 nuclei or cells.

57. The method of claim 52, wherein compartments of the third plurality of compartments comprise from 50 to 100,000,000 nuclei or cells.

58. The method of claim 48, wherein the contacting comprises contacting each subset with two transposome complexes, wherein one transposome complex comprises a first transposase comprising a first universal sequence and a second transposome complex comprises a second transposase comprising a second universal sequence, wherein the contacting further comprises conditions suitable for incorporation of the first universal sequence and the second universal sequence into DNA nucleic acids resulting in double stranded DNA nucleic acids comprising the first and second universal sequences.

59. The method of any one of claims 48, 49, or 50, wherein the adding of the compartment specific index sequence comprises a two-step process of adding a nucleotide sequence comprising a universal sequence to the nucleic acids, and then adding the compartment specific index sequence to the nucleic acids.

60. The method of claim 48, further comprising obtaining the indexed nucleic acids from the pooled indexed nuclei or cells, thereby producing a sequencing library from the plurality of nuclei or cells.

61. The method of claim 49, further comprising obtaining the dual-indexed nucleic acids from the pooled dual-indexed nuclei or cells, thereby producing a sequencing library from the plurality of nuclei or cells.

62. The method of claim 50, further comprising obtaining the triple-indexed nucleic acids from the pooled triple-indexed nuclei or cells, thereby producing a sequencing library from the plurality of nuclei or cells.

63. The method of any one of claims 60-62, further comprising:
providing a surface comprising a plurality of amplification sites,
wherein the amplification sites comprise at least two populations of attached single stranded capture oligonucleotides having a free 3′ end, and
contacting the surface comprising amplification sites with the nucleic acid fragments comprising one, two, or three index sequences under conditions suitable to produce a plurality of amplification sites that each comprise a clonal population of amplicons from an individual fragment comprising a plurality of indexes.

64. A method for preparing a nucleic acid library comprising:
(a) providing a plurality of samples, wherein each sample comprises a plurality of cells or nuclei, wherein the plurality of cells or nuclei of each sample are present in one or more separate compartments;
(b) contacting the plurality of nuclei or cells with a transposome complex comprising a transposase and a universal sequence and with the proviso that the transposome complex does not comprise an index sequence, wherein the contacting further comprises conditions suitable for incorporation of the universal sequence into nucleic acids;
(c) adding a first index sequence to the nucleic acids of each separate compartment;
(d) combining the cells or nuclei of the separated compartments;
(e) distributing the cells or nuclei into a plurality of compartments; and
(f) adding a second index sequence to the nucleic acids of the plurality of compartments.

65. The method of claim 64, wherein the first index sequence, the second index sequence, or the combination thereof, are added by ligation, primer extension, hybridization, amplification, or a combination thereof.

66. The method of claim 64 or 65, wherein steps (d)-€ are repeated to add a third or more index sequences to the cells or nuclei of the plurality of compartments.

67. The method of any one of claims 64 or 65, wherein the plurality of nuclei or cells are fixed.

68. The method of any one of claims 64 or 65, further comprising an amplification of indexed nucleic acids after step (c) or step (f).

69. The method of any one of claims 64 or 65, further comprising step (g) combining the nucleic acids of the plurality of compartments and determining the sequence of the nucleic acids.

70. The method of claim 64, further comprising performing a sequencing procedure to determine the nucleotide sequences for the nucleic acids.

71. A method for sequencing a single cell or nucleus comprising:
(a) uniquely indexing nucleic acids of each cell or nuclei in a sample, thereby generating an indexed library for each cell or nuclei;
(b) using a biological feature to identify one or more indexed libraries of interest from step (a);
(c) enriching the indexed libraries of interest of step (b) thereby generating an enriched library; and
(d) sequencing the enriched library from step (c).

72. The method of claim 71, wherein the libraries are derived from DNA, RNA, or protein of the cells or nuclei.

73. The method of any one of claims 71 or 72, wherein the biological feature is DNA, RNA, or protein or a combination thereof.

74. The method of any one of claims 71 or 72, wherein the uniquely indexing in step (a) comprises associating at least two different indexes to the nucleic acids of the cells or nuclei.

75. The method of claim 74, wherein the at least two different indexes are a contiguous index.

76. The method of any one of claims 71 or 72, wherein the enriched library is generated through positive enrichment.

77. The method of claim 76, wherein the positive enrichment comprises amplification.

78. The method of claim 76, wherein the positive enrichment comprises a capture agent.

79. The method of claim 76, wherein the positive enrichment comprises a solid support.

80. The method of claim 76, wherein the enriched library is generated through negative enrichment.

81. The method of any one of claims 71 or 72, wherein the identifying the indexed library of interest in step (c) comprises sequencing the indexes.

82. A method for sequencing a single cell or nucleus comprising:
(a) providing a sample, wherein the sample comprises a plurality of nuclei or cells;
(b) associating a first index on each nucleus or cell in the sample;
(c) dividing the sample into a plurality of compartments;
(d) associating a second index on each nuclei or cell of the plurality of compartments;
(e) pooling the plurality of compartments;
(f) sequencing the pooled compartments;
(g) identifying a combination of first and second indexes associated with a biological feature;
(h) enriching the biological feature from the pooled compartments using the identified combination of first and second indexes from step (g).

83. A kit containing:
(a) a plurality of transposome complexes, wherein each transposome complex comprises a transposase and a transposon sequence, wherein the transposon sequence is not indexed;
(b) a first plurality of index oligonucleotides, wherein the first plurality of index oligonucleotides comprises oligonucleotides having at least two different sequences; and
(c) a ligase enzyme for use with the index oligonucleotides.

84. The kit of claim 83, further comprising a second plurality of index oligonucleotides, wherein the second plurality of index oligonucleotides comprises oligonucleotide having different sequences from the first plurality of index oligonucleotides.

85. The kit of claim 83, further comprising a third plurality of index oligonucleotides, wherein the third plurality of index oligonucleotides comprises oligonucleotide having different sequences from the first plurality of index oligonucleotides and the second plurality of index oligonucleotides.
